(19) United States
(12) Patent Application Publication
(10) Pub. No.: US 2011/0319418 A1
(43) Pub. Date: Dec. 29, 2011

(54) PROKINETICIN 1 RECEPTOR ANTAGONISTS FOR THE TREATMENT OF PAIN

(76) Inventors: Christopher M. Flores, Lansdale, PA (US); Paul R. Wade, Philadelphia, PA (US)

(21) Appl. No.: 13/169,493

(22) Filed: Jun. 27, 2011

Related U.S. Application Data
(60) Provisional application No. 61/359,079, filed on Jun. 28, 2010.

(51) Int. Cl. A61K 31/53; C07D 239/545; A61P 25/00; C07D 239/54; C07D 239/60; A61K 31/513; C07D 251/46; C07D 403/12

(52) U.S. Cl. 514/245; 544/211; 544/312; 544/212; 544/314; 544/302; 514/274; 514/241

(57) ABSTRACT

Disclosed are compounds, compositions and methods for treating pain, including inflammatory, visceral, and acute pain. Such compounds are represented by Formula (I) as follows:

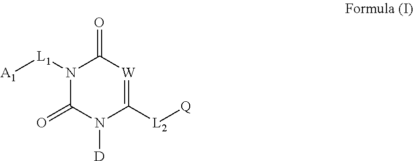

wherein A₁, L₁, D, L₂, and Q are defined herein.

### CROSS REFERENCE TO RELATED U.S. APPLICATION DATA

The present application is derived from and claims priority to provisional application U.S. Ser. No. 61/359,079, filed Jun. 28, 2010, which is herein incorporated by reference in its entirety.

The nonprovisional application entitled, Prokineticin 1 Receptor Antagonists, U.S. Nonprovisional application Ser. No. 11/375,407, filed on Mar. 14, 2006, is hereby incorporated by reference in its entirety.

### STATEMENT REGARDING FEDERALLY SPONSORED RESEARCH OR DEVELOPMENT

The research and development of the invention described below was not federally sponsored.

### FIELD OF THE INVENTION

The present invention is directed to the use of a compound of Formula (I), as herein defined, for the treatment, amelioration, and/or prevention of pain, including inflammatory pain, visceral pain, and acute pain, in a subject, including a mammal and/or human, in need thereof.

### BACKGROUND OF THE INVENTION

Sensitization is an important property of pain signaling. Painful stimuli can induce central (spinal and supraspinal) and peripheral (nociceptor) sensitization. Both types of sensitization play a role in inflammatory diseases, the single greatest cause of chronic pain.

Prokineticin-1 and Prokineticin-2, PKR1 and PKR2 respectively, are naturally occurring peptide agonists of two G-protein-coupled receptors (GPCRs) and are expressed in neurons in the central nervous system (“CNS”) and peripheral nervous system. Many dorsal root ganglion cells expressing PKRs also express transient receptor potential vanilloid receptor-1 (TRPV1). It has been suggested that PKR1 plays a modulatory role in acute nociception and inflammatory pain through a pharmacological interaction with TRPV1 in nociceptor activation and sensitization. Moreover, PKR1 and PKR2 (Lin, DCH et al. J. Biol. Chem. 2002, 277, p 19276-19280) and their activation by peptides belonging to the Bv8/EG-VEGF (endocrine gland-derived vascular endothelial growth factor)-PK (prokineticin) family suggest an additional novel mechanism of peripheral nociceptor activation and sensitization (Negri et al., Br. J. Pharmacol. 2002, 146, p. 1147-1154).

It is suggested that prokineticin 1 receptor antagonists would be useful for the treatment and prevention of various mammalian pain states, including inflammatory pain, visceral pain, and acute pain.

It is an object of the present invention to provide prokineticin 1 receptor antagonists. It is also an object of the invention to provide a method of treating, ameliorating or preventing pain by the administration of a compound of Formula (I). And, it is an object of the invention to provide a pharmaceutical composition comprising a compound of Formula (I), useful for treating, ameliorating or preventing pain.

### SUMMARY OF THE INVENTION

The present invention is directed to a method for treating, ameliorating, or preventing pain; comprising, consisting of, and/or consisting essentially of administering to a subject in need thereof, a therapeutically effective amount of a compound of Formula (I)

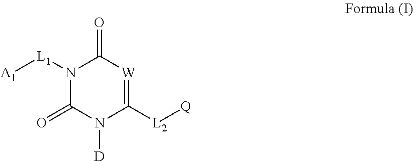

or an enantiomer, diastereomer, solvate, or pharmaceutically acceptable salt thereof;

wherein:
A₁ is hydrogen; aryl; heteroaryl; C₅₋₈cycloalkyl; or heterocyclyl; provided that A₁ is other than piperidin-4-yl, N-t-butoxycarbonyl-piperidin-4-yl, or N-methyl-piperidin-3-yl; and wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl;
L₁ is —(CH₂)ᵣ— or —CH₂CH₂X(CH₂)ₛ—, optionally substituted with one to three substituents independently selected from the group consisting of C₁₋₆alkyl, C₂₋₆alkenyl, C₂₋₆alkynyl, and halogen; provided that when A₁ is hydrogen, r is greater than or equal to 4;
r is an integer of 1 to 5;
s is an integer of 1 to 3;
X is O or S;
D is —P-A₂; wherein when A₂ is hydrogen, P is —(CH₂)₄₋₆—, and when A₂ is other than hydrogen, P is —(CH₂)₁₋₂— or —CH₂CH═CH—;
A₂ is hydrogen; benzodioxalyl; heteroaryl other than unsubstituted pyridin-2-yl; C₃₋₈cycloalkyl; or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; wherein benzodioxalyl, heteroaryl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy;
provided that no more than two substituents on A₂ are aryl(C₁₋₆)alkoxy, phenyl, or a non fused C₃₋₆cycloalkyloxy;
provided that when A₁ is unsubstituted phenyl and L₂ is —X₁—CH(R^{x})—(CR^{y}R^{z})— wherein X₁ is NH, and R^{x}, R^{y}, and R^{z} are each hydrogen, A₂ is other than unsubstituted phenyl; phenyl substituted with aryl(C₁₋₆)alkoxy or phenyl; or phenyl substituted at the meta position with cyano;
and, further provided that when A₁ is unsubstituted phenyl and L₂ is —X₁—CH(R^{x})—(CR^{y}R^{z})₂ — wherein X₁ is NH and R^{x}, R^{y}, and R^{z} are each hydrogen, A₂ is other than phenyl substituted with methoxy;
and, provided that when A₁ is 3,4-dichloro-phenyl and P is —CH₂—, A₂ is other than phenyl substituted at the meta position with trifluoromethyl or trifluoromethoxy;
and, further provided that when A₁ is 3,4-dichloro-phenyl and P is —(CH₂)₂—, A₂ is other than 4-methoxy-phenyl;
W is N or C(R_{W}); wherein R_{W} is H or C₁₋₂alkyl;
L₂ is a bivalent radical selected from the group consisting of
   pyrrolidinyl or piperidinyl attached to the triazine ring of Formula (I) via its nitrogen atom, wherein said pyrrolidinyl or piperidinyl is substituted on a carbon atom with —(CH₂)₀₋₂—;
   —NH—C₅₋₇cycloalkyl-(CH₂)₀₋₂—; such that when C₅₋₇cycloalkyl is cyclohexyl, Q is attached at either the 2- or cis-4-position relative to the position of —NH—;
   —X₁—(CH₂)ᵤ—X₂—(CH₂)ᵥ—; wherein u is an integer of 1 to 3; and wherein v is an integer of 1 to 4; provided that when X₁ is a direct bond and W is C(R_{w}), then u is 1 and v is 2 to 4;
   —X₂—(CH₂)₀₋₄—;
   —X₁—(CH₂)₂₋₃—X₃—(CH₂)₂₋₃—;
   —NH(CH₂)₁₋₄C(═O)—, provided that at least one of R^{b}, R^{c}, or R^{d} is other than hydrogen and m is 0;
   —NHC(═O)—(CH₂)₁₋₄—;
   —C(═O)NH(CR^{y}R^{z})₂₋₅—;
   and
   —X₁—CH(R^{x})—(CR^{y}R^{z})₁₋₅—; such that when X₁ is a direct bond and W is C(R_{w}), then R^{x} is hydrogen;
wherein X₁ is —NH—, O, S, or a direct bond, such that X₁ is other than O when W is N;
   X₂ is —CH═CH—;
   X₃ is O, S, NH, or C═O;
   R^{x}, R^{y}, and R^{z} are independently H or C₁₋₄alkyl;
and provided that L₂ in any instance does not exceed 7 atoms in length;
and further provided that when L₂ is —X₂—(CH₂)₀₋₄— or —C(═O)NH(CR^{y}R^{z})₂₋₅—, then R_{W} is hydrogen;
Q is —(O)ₘN(R^{a})-G; and m is 0 or 1;
G is —C(═NR^{b})NR^{c}R^{d};
R^{a} and R^{d} are independently hydrogen, C₁₋₆alkyl, C₂₋₆alkenyl, or C₃₋₆alkynyl, wherein substituents of R^{a} and R^{d} other than hydrogen are optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
R^{b} is hydrogen, C₁₋₆alkyl, C₂₋₆alkenyl, C₃₋₆alkynyl, C₂₋₆alkoxycarbonyl, or cyano; or, R^{b} and R^{c} are taken together with the atoms to which they are attached to form a 5-8 membered monocyclic ring optionally substituted with oxo;
R^{c} is hydrogen, C₁₋₁₀alkyl, C₂₋₁₀alkenyl, C₃₋₁₀alkynyl, C₃₋₇cycloalkyl, adamantyl, amino, C₁₋₆alkylamino, di(C₁₋₆alkyl)amino, C₁₋₆alkylcarbonyl, C₁₋₆alkoxycarbonyl, arylcarbonyl, heteroarylcarbonyl, heterocyclylcarbonyl, aryl, heteroaryl, or heterocyclyl; wherein C₁₋₁₀alkyl, C₂₋₁₀alkenyl, and C₂₋₁₀alkynyl are optionally substituted with one to three substituents independently selected from the group consisting of hydroxy, C₁₋₆alkoxy, trifluoromethyl, aryl, heteroaryl, and heterocyclyl; and wherein any aryl- or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, aminocarbonyl, C₁₋₆alkylaminocarbonyl, di(C₁₋₆alkyl)aminocarbonyl, C₁₋₆alkoxycarbonylamino, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl, nitro, methylthio, hydroxy, and cyano; or, R^{c} and R^{d} are taken together with the atoms to which they are attached to form a 5-8 membered monocyclic ring that optionally includes 1 to 2 O or S heteroatoms within the ring, and said ring is optionally substituted with oxo;
with the proviso that in any instance, only one ring optionally exists between R^{a} and R^{b}, R^{b} and R^{c}, or R^{c} and R^{d}.

The present invention is further directed to the use of a compound of Formula (I) as herein defined for the preparation of a medicament or a pharmaceutical composition for the treatment, amelioration and/or prevention of pain, including inflammatory, visceral, and acute pain, in a subject in need thereof.

### DETAILED DESCRIPTION OF THE INVENTION

As used herein, the following terms are intended to have the following meanings:

“C_{a-b}” (where a and b are integers) refers to a radical containing from a to b carbon atoms inclusive. For example, C₁₋₃ denotes a radical containing 1, 2 or 3 carbon atoms.

With reference to substituents, the term “independently” means that when more than one of such substituent is possible, such substituents may be the same or different from each other. Therefore, designated numbers of carbon atoms (e.g. C₁₋₈) shall refer independently to the number of carbon atoms in an alkyl or cycloalkyl moiety or to the alkyl portion of a larger substituent in which alkyl appears as its prefix root.

As used herein, unless otherwise noted, “alkyl” whether used alone or as part of a substituent group refers to straight and branched carbon chains having 1 to 8 carbon atoms or any number within this range. The term “alkoxy” refers to an —Oalkyl substituent group, wherein alkyl is as defined supra. Similarly, the terms “alkenyl” and “alkynyl” refer to straight and branched carbon chains having 2 to 8 carbon atoms or any number within this range, wherein an alkenyl chain has at least one double bond in the chain and an alkynyl chain has at least one triple bond in the chain. An alkyl and alkoxy chain may be substituted on a carbon atom. In substituent groups with multiple alkyl groups such as (C₁₋₆alkyl)₂amino- the C₁₋₆alkyl groups of the dialkylamino may be the same or different.

“Halogenated alkyl” refers to a saturated branched or straight chain alkyl radical derived by removal of 1 hydrogen atom from the parent alkyl; the parent alkyl chain contains from 1 to 8 carbon atoms with 1 or more hydrogen atoms substituted with halogen atoms up to and including substitution of all hydrogen atoms with halogen. Preferred halogenated alkyl groups include include trifluoromethyl substituted alkyls and perfluorinated alkyls; more preferred fluorinated alkyls include trifluoromethyl.

“Halogenated alkoxy” refers to a radical derived from a halogenated alkyl, radical attached to an oxygen atom with the oxygen atom having one open valence for attachment to a parent structure.

The term “cycloalkyl” refers to saturated or partially unsaturated, moncyclic or polycyclic hydrocarbon rings of from 3 to 20 carbon atom members (preferably from 3 to 14 carbon atom members). Examples of such rings include, and are not limited to, cyclopropyl, cyclobutyl, cyclopentyl, cyclohexyl, cycloheptyl or adamantyl. The term cycloalkyl includes a cycloalkyl ring fused to a benzene ring (benzo fused cycloalkyl), a 5 or 6 membered heteroaryl ring (containing one of O, S or N and, optionally, one additional nitrogen) to form a heteroaryl fused cycloalkyl.

The term “heterocyclyl” refers to a nonaromatic cyclic ring of 5 to 10 members in which 1 to 4 members are nitrogen or a nonaromatic cyclic ring of 5 to 10 members in which zero, one or two members are nitrogen and up to two members is oxygen or sulfur; wherein, optionally, the ring contains zero, one or two unsaturated bonds. The term heterocyclyl includes a heterocyclyl ring fused to a benzene ring (benzo fused heterocyclyl), a 5 or 6 membered heteroaryl ring (containing one of O, S or N and, optionally, one additional nitrogen), a 5 to 7 membered cycloalkyl or cycloalkenyl ring, a 5 to 7 membered heterocyclyl ring (of the same definition as above but absent the option of a further fused ring) or fused with the carbon of attachment of a cycloalkyl, cycloalkenyl or heterocyclyl ring to form a spiro moiety. For instant compounds of the invention, the carbon atom ring members that form the heterocyclyl ring are fully saturated. Other compounds of the invention may have a partially saturated heterocyclyl ring. Additionally, heterocyclyl includes a heterocyclic ring bridged to form bicyclic rings. Preferred partially saturated heterocyclyl rings may have from one to two double bonds. Such compounds are not considered to be fully aromatic and are not referred to as heteroaryl compounds. Examples of heterocyclyl groups include, and are not limited to, pyrrolinyl (including 2H-pyrrole, 2-pyrrolinyl or 3-pyrrolinyl), pyrrolidinyl, 2-imidazolinyl, imidazolidinyl, 2-pyrazolinyl, pyrazolidinyl, piperidinyl, morpholinyl, thiomorpholinyl and piperazinyl.

The term “aryl” refers to an unsaturated, aromatic monocyclic ring of 6 carbon members or to an unsaturated, aromatic polycyclic ring of from 10 to 14 carbon members. Examples of such aryl rings include, and are not limited to, phenyl, naphthalenyl or anthracenyl. Preferred aryl groups for the practice of this invention are phenyl and naphthalenyl.

The term “heteroaryl” refers to an aromatic ring of 5 or 6 members wherein the ring consists of carbon atoms and has at least one heteroatom member. Suitable heteroatoms include nitrogen, oxygen or sulfur. In the case of 5 membered rings, the heteroaryl ring contains one member of nitrogen, oxygen or sulfur and, in addition, may contain up to three additional nitrogens. In the case of 6 membered rings, the heteroaryl ring may contain from one to three nitrogen atoms. For the case wherein the 6 membered ring has three nitrogens, at most two nitrogen atoms are adjacent. The term heteroaryl includes a heteroaryl ring fused to a benzene ring (benzo fused heteroaryl), a 5 or 6 membered heteroaryl ring (containing one of O, S or N and, optionally, one additional nitrogen), a 5 to 7 membered cycloalkyl ring or a 5 to 7 membered heterocyclic ring (as defined supra but absent the option of a further fused ring). Examples of heteroaryl groups include, and are not limited to, furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl or pyrazinyl; fused heteroaryl groups include indolyl, isoindolyl, indolinyl, benzofuryl, benzothienyl, indazolyl, benzimidazolyl, benzthiazolyl, benzoxazolyl, benzisoxazolyl, benzothiadiazolyl, benzotriazolyl, quinolizinyl, quinolinyl, isoquinolinyl or quinazolinyl.

The term “arylalkyl” means an alkyl group substituted with an aryl group (e.g., benzyl, phenethyl). Similarly, the term “arylalkoxy” indicates an alkoxy group substituted with an aryl group (e.g., benzyloxy).

The term “halogen” refers to fluorine, chlorine, bromine and iodine. Substituents that are substituted with multiple halogens are substituted in a manner that provides compounds, which are stable.

The term “oxo” whether used alone or as part of a substituent group refers to an O═ to either a carbon or a sulfur atom. For example, phthalimide and saccharin are examples of compounds with oxo substituents.

Whenever the term “alkyl” or “aryl” or either of their prefix roots appear in a name of a substituent (e.g., arylalkyl, alkylamino) it shall be interpreted as including those limitations given above for “alkyl” and “aryl.” Designated numbers of carbon atoms (e.g., C₁-C₆) shall refer independently to the number of carbon atoms in an alkyl moiety or to the alkyl portion of a larger substituent in which alkyl appears as its prefix root. For alkyl, and alkoxy substituents the designated number of carbon atoms includes all of the independent member included in the range specified individually and all the combination of ranges within in the range specified. For example C₁₋₆ alkyl would include methyl, ethyl, propyl, butyl, pentyl and hexyl individually as well as sub-combinations thereof (e.g. C₁₋₂, C₁₋₃, C₁₋₄, C₁₋₅, C₂₋₆, C₃₋₆, C₄₋₆, C₅₋₆, C₂₋₅, etc.).

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, the term “acyl” refers to alkylcarbonyl substituents.

Throughout this disclosure, the terminal portion of the designated side chain is described first, followed by the adjacent functionality toward the point of attachment. Thus, for example, a “phenyl(C₁₋₆)alkylaminocarbonyl(C₁₋₆)alkyl” substituent refers to a group of the formula

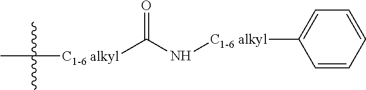

Unless otherwise noted, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “subject” as used herein, refers to an animal, preferably a mammal, most preferably a human, who has been the object of treatment, observation or experiment.

The term “therapeutically effective amount” means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease, syndrome, condition or disorder being treated.

The term “composition” is intended to encompass a product comprising the specified ingredients in therapeutically effective amounts, as well as any product that results, directly or indirectly, from combinations of the specified ingredients in the specified amounts.

As used herein, unless otherwise noted, the terms “treating”, “treatment”, “ameliorating” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the terms “preventing” and “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical professional to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

As used herein, unless otherwise noted, the term “antagonist” is used to refer to a compound capable of producing, depending on the circumstance, a functional antagonism of the prokinetin receptor 1, including, but not limited to, competitive antagonists, non-competitive antagonists, desensitizing agonists, and partial agonists.

As used herein, unless otherwise noted, the term “affect” or “affected” (when referring to a disease, syndrome, condition or disorder that is affected by inhibition of the PK1 receptor) shall imply a reduction in the frequency and/or severity of one or more symptoms or manifestations of said disease, syndrome, condition or disorder; and/or imply the prevention of the development of one or more symptoms or manifestations of said disease, syndrome, condition or disorder or the development of the disease, condition, syndrome or disorder.

The compounds of Formula (I) are useful in methods for treating, ameliorating and/or preventing pain or a disease, a syndrome, a condition or a disorder that causes such pain by the antagonism of prokineticin 1 receptor. Such methods comprise, consist of and/or consist essentially of administering to a subject, including an animal, a mammal, and a human in need of such treatment, amelioration and/or prevention, a therapeutically effective amount of a compound of Formula (I), or an enantiomer, diastereomer, solvate or pharmaceutically acceptable salt thereof More particularly, the compounds of Formula (I) are useful for treating, ameliorating and/or preventing inflammatory pain, visceral pain and/or acute pain, comprising administering to a subject in need thereof a therapeutically effective amount of a compound of Formula (I), as herein defined.

Examples of inflammatory pain include pain due to a disease, condition, syndrome or disorder, including inflammatory bowel disease, visceral pain, migraine, post operative pain, osteoarthritis, rheumatoid arthritis, back pain, lower back pain, joint pain, abdominal pain, chest pain, labor pain, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

The term visceral pain, as used herein, refers to pain caused by inflammation of serous surfaces, distention of viscera and inflammation or compression of peripheral nerves. Examples of visceral pain include, but are not limited to, abdominal pain, chest pain, pelvic pain, including vulvodynia as well as pain associated with labor or menstruation, and/or pain associated with inflammatory bowel disease, irritable bowel syndrome, neurogenic bladder, interstitial cystitis, cholecystitis, pancreatitis and urinary tract infection.

Acute pain, as used herein, refers to pain that comes on quickly, can be severe, but is of relatively short duration. Examples of acute pain include, but are not limited to, post-operative pain, post-surgical pain, toothache, burn, sunburn, insect/animal bites and stings, headache and/or any pain associated with acute trauma or injury.

In an embodiment, the present invention is directed to a method for treating, ameliorating, or preventing pain; comprising, consisting of, and/or consisting essentially of administering to a subject in need thereof a therapeutically effective amount of a compound of Formula (I)

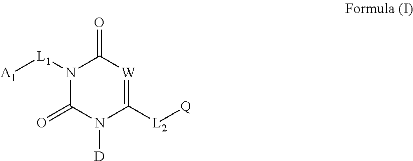

or an enantiomer, diastereomer, solvate, or pharmaceutically acceptable salt thereof;

wherein:
a) A₁ is hydrogen; aryl; heteroaryl; or C₅₋₈cycloalkyl; wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl;
b) A₁ is hydrogen; aryl; heteroaryl; C₅₋₈cycloalkyl; or heterocyclyl; provided that A₁ is other than piperidin-4-yl, N-t-butoxycarbonyl-piperidin-4-yl, or N-methyl-piperidin-3-yl; and wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, cyano, hydroxy, aminocarbonyl, C₁₋₆alkylaminocarbonyl, di(C₁₋₆alkyl)aminocarbonyl, and C₁₋₆alkylcarbonyl;
c) A₁ is hydrogen; aryl; heteroaryl; C₅₋₈cycloalkyl; or heterocyclyl other than piperidinyl; wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, cyano, hydroxy, aminocarbonyl, C₁₋₆alkylaminocarbonyl, di(C₁₋₆alkyl)aminocarbonyl, and C₁₋₆alkylcarbonyl;
d) A₁ is hydrogen, substituted phenyl, benzofuranyl, furanyl, thiazolyl, thiophenyl, or cyclopentyl; wherein substituents of A₁ other than hydrogen are optionally substituted and phenyl is substituted with one to two substituents independently selected from the group consisting of C₁₋₄alkyl, C₁₋₄alkoxy, halogen, nitro, halogenated C₁₋₄alkyl, halogenated C₁₋₄alkoxy, methylthio, C₁₋₄alkoxycarbonyl, amino, cyano, hydroxy, aminocarbonyl, and C₁₋₄alkylcarbonyl;
e) A₁ is substituted phenyl, benzofuranyl, thiazolyl, or thiophenyl; wherein phenyl is substituted with, and benzofuranyl, thiazolyl, and thiophenyl are optionally substituted with one to two substituents independently selected from the group consisting of C₁₋₄alkyl, C₁₋₄alkoxy, halogen, nitro, halogenated C₁₋₄alkyl, halogenated C₁₋₄alkoxy, methylthio, amino, cyano, and C₁₋₄alkylcarbonyl;
f) A₁ is phenyl or benzofuranyl; wherein phenyl is substituted at either the para-position or meta and para-positions with one to two substituents independently selected from the group consisting of ethyl, methoxy, fluoro, chloro, nitro, difluoromethoxy, and methylthio;
g) L₁ is —(CH₂)ᵣ—, optionally substituted with one to three substituents independently selected from the group consisting of C₁₋₆alkyl, C₂₋₆alkenyl, C₂₋₆alkynyl, and halogen; provided that when A₁ is hydrogen, r is greater than or equal to 4;
h) L₁ is —(CH₂)ᵣ—, optionally substituted with a substituent selected from the group consisting of C₁₋₄alkyl, C₂₋₄alkenyl, and C₂₋₄alkynyl, provided that r is 1 to 3 when A₁ is other than hydrogen; or r is greater than or equal to 4 when A₁ is hydrogen;
i) L₁ is —(CH₂)ᵣ— optionally substituted with a substituent selected from the group consisting of methyl and allyl, provided that r is 1 to 3 when A₁ is other than hydrogen;
j) L₁ is —CH₂— optionally substituted with methyl or allyl;
k) P is —CH₂—
l) A₂ is hydrogen, heteroaryl other than unsubstituted pyridin-2-yl, C₃₋₈cycloalkyl, or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, cyano, hydroxy, nitro, aminocarbonyl, C₁₋₆alkylcarbonylamino, and a non fused C₃₋₆cycloalkyloxy; wherein heteroaryl other than unsubstituted pyridin-2-yl and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, cyano, hydroxy, nitro, aminocarbonyl, C₁₋₆alkylcarbonylamino, and a non fused C₃₋₆cycloalkyloxy;
   provided that no more than two substituents on A₂ are aryl(C₁₋₆)alkoxy, phenyl, or a non fused C₃₋₆cycloalkyloxy;
   provided that when A₁ is unsubstituted phenyl and L₂ is —X₁—CH(R^{x})—(CR^{y}R^{z})— wherein X₁ is NH and R^{x}, R^{y}, and R^{z} are each hydrogen, A₂ is other than unsubstituted phenyl; phenyl substituted with aryl(C₁₋₆)alkoxy or phenyl; or phenyl substituted at the meta position with cyano;
   and, further provided that when A₁ is unsubstituted phenyl and L₂ is —X₁—CH(R^{x})—(CR^{y}R^{z})₂ — wherein X₁ is NH and R^{x}, R^{y}, and R^{z} are each hydrogen, A₂ is other than phenyl substituted with methoxy;
   and, provided that when A₁ is 3,4-dichloro-phenyl and P is —CH₂—, A₂ is other than phenyl substituted at the meta position with trifluoromethyl or trifluoromethoxy;
   and, further provided that when A₁ is 3,4-dichloro-phenyl and P is —(CH₂)₂—, A₂ is other than 4-methoxy-phenyl;
   in addition, when A₂ is hydrogen, P is —(CH₂)₄₋₆—, and when A₂ is other than hydrogen, P is —(CH₂)₁₋₂— or —CH₂CH═CH—;
m) A₂ is heteroaryl other than unsubstituted pyridin-2-yl, a non fused C₃₋₈cycloalkyl, or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, C₁₋₆alkylthio, C₁₋₆alkoxycarbonyl, amino, hydroxy, nitro, aminocarbonyl, C₁₋₆alkylcarbonylamino, and a non fused C₃₋₆cycloalkyloxy; wherein heteroaryl other than unsubstituted pyridin-2-yl and a non fused C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, C₁₋₆alkylthio, C₁₋₆alkoxycarbonyl, amino, hydroxy, nitro, aminocarbonyl, C₁₋₆alkylcarbonylamino, and a non fused C₃₋₆cycloalkyloxy; provided that no more than two substituents on A₂ are non fused C₃₋₆cycloalkyloxy;
   provided that when A₁ is unsubstituted phenyl and L₂ is —X₁—CH(R^{x})—(CR^{y}R^{z})— wherein X₁ is NH and R^{x}, R^{y}, and R^{z} are each hydrogen, A₂ is other than unsubstituted phenyl;
   and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
   and, provided that when A₁ is 3,4-dichloro-phenyl, A₂ is other than phenyl substituted at the meta position with trifluoromethyl or trifluoromethoxy;
   and, further provided that when A₁ is 3,4-dichloro-phenyl and P is —(CH₂)₂—, A₂ is other than 4-methoxy-phenyl;
n) A₂ is furanyl, pyridin-3-yl, pyridin-4-yl, or phenyl optionally substituted at the meta and para positions with one to three substituents independently selected from the group consisting of C₁₋₄alkyl, C₁₋₄alkoxy, halogen, halogenated C₁₋₃alkoxy, C₁₋₃alkylthio, hydroxy, amino, aminocarbonyl, C₁₋₃alkylcarbonylamino, and a non fused C₃₋₆cycloalkyloxy; and wherein furanyl, pyridin-3-yl, and pyridin-4-yl are optionally substituted with one to three substituents independently selected from the group consisting of C₁₋₄alkyl, C₁₋₄alkoxy, halogen, halogenated C₁₋₃alkoxy, C₁₋₃alkylthio, hydroxy, amino, aminocarbonyl, C₁₋₃alkylcarbonylamino, and a non fused C₃₋₆cycloalkyloxy; provided that no more than two substituents on A₂ are non fused C₃₋₆cycloalkyloxy;
   provided that when A₁ is unsubstituted phenyl and L₂ is —X₁—CH(R^{x})—(CR^{y}R^{z})— wherein X₁ is NH and R^{x}, R^{y}, and R^{z} are each hydrogen, A₂ is other than unsubstituted phenyl;
   and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
   and, provided that when A₁ is 3,4-dichloro-phenyl, A₂ is other than phenyl substituted in the meta position with trifluoromethoxy;
o) A₂ is pyridin-3-yl pyridin-4-yl, or phenyl optionally substituted at the meta and para positions with one to two substituents independently selected from the group consisting of methyl, ethyl, methoxy, ethoxy, isopropyloxy, trifluoromethoxy, difluoromethoxy, hydroxy, aminocarbonyl, and methylcarbonylamino; wherein pyridin-3-yl and pyridin-4-yl are optionally substituted with one to two substituents independently selected from the group consisting of methyl, ethyl, methoxy, ethoxy, isopropyloxy, trifluoromethoxy, difluoromethoxy, hydroxy, aminocarbonyl, and methylcarbonylamino;
   provided that when A₁ is unsubstituted phenyl and L₂ is —X₁—CH(R^{x})—(CR^{y}R^{z})— wherein X₁ is NH and R^{x}, R^{y}, and R^{z} are each hydrogen, A₂ is other than unsubstituted phenyl;
   and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
   and, provided that when A₁ is 3,4-dichloro-phenyl, A₂ is other than phenyl substituted in the meta position with trifluoromethoxy;
p) A2 is phenyl substituted at the para position with a substituent selected from the group consisting of methoxy, ethoxy, isopropyloxy, difluoromethoxy, hydroxy, and aminocarbonyl; or A₂ is pyridin-3-yl or pyridin-4-yl substituted with methoxy;
q) W is N or C(R_{w}) wherein R_{w} is H;
r) L₂ is a bivalent radical selected from the group consisting of
   —NH—C₅₋₇cycloalkyl-(CH₂)₀₋₂—; provided that when C₅₋₇cycloalkyl is cyclohexyl, Q is attached at either the 2- or cis-4-position relative to the position of —NH—;
   —X₂—(CH₂)₀₋₄—;
   —X₁—(CH₂)₂₋₃—X₃—(CH₂)₂₋₃—;
   —NH(CH₂)₁₋₄C(═O)— provided that at least one of R^{b}, R^{c}, or R^{d} is other than hydrogen and m is 0;
   —NHC(═O)—(CH₂)₁₋₄—;
   —C(═O)NH(CR^{y}R^{z})₂₋₅—;
   and
   —X₁—CH(R^{x})—(CR^{y}R^{z})₁₋₅—; such that when X₁ is a direct bond and W is C(R_{w}), then R^{x} of CH(R^{x}) is hydrogen;
   wherein X₁ is —NH—, O, S, or a direct bond; such that X₁ is other than O when W is N;
   X₂ is —CH═CH—;
   X₃ is O, S, NH, or C═O;
   R^{x}, R^{y}, and R^{z} are independently H or C₁₋₄alkyl;
   and provided that L₂ in any instance does not exceed 7 atoms in length; and further provided that when L₂ is —X₂—(CH₂)₀₋₄— or —C(═O)NH(CR^{y}R^{z})₂₋₅—, then R_{W} is hydrogen;
s) L₂ is a bivalent radical selected from the group consisting of
   —NH—C₅₋₆cycloalkyl-(CH₂)₀₋₂—; provided that when C₅₋₆cycloalkyl is cyclohexyl, Q is attached at either the 2- or cis-4-position relative to the position of —NH—;
   —X₁—CH(R^{x})—(CR^{y}R^{z})₁₋₅—, wherein X₁ is —NH—, O, or S and R^{x}, R^{y}, and R^{z} are each hydrogen; such that X₁ is other than O when W is N;
   —C(═O)NH(CH₂)₂—;
   and
   —X₁—(R,R—CH(R^{x})CR^{y}(R^{z}))—; wherein X₁ is —NH—, and R^{x} and R^{z} are methyl, and R^{y} is hydrogen;
   provided that when L₂ is —C(═O)NH(CH₂)₂—, then R_{W} is hydrogen;
t) L₂ is a bivalent radical selected from the group consisting of
   —NH-cyclohexyl-(CH₂)₀₋₂— and Q is attached at either the 2- or cis-4-position relative to the position of —NH—;
   —X₁—CH(R^{x})—(CR^{y}R^{z})₁₋₅; wherein X₁ is —NH— or S; and R^{x}, R^{y}, and R^{z} are each hydrogen;
   and
   —X₁—(R,R—CH(R^{x})CR^{y}(R^{z}))—; wherein X₁ is —NH—, and R^{x} and R^{z} are methyl, and R^{y} is hydrogen;
u) L₂ is a bivalent radical selected from the group consisting of
   —NH-cyclohexyl-(CH₂)₀₋₂— and Q is attached at either the 2- or cis-4-position relative to the position of —NH—;
   —X₁—CH(R^{x})—(CR^{y}R^{z})—; wherein X₁ is —NH— or S and R^{x}, R^{y}, and R^{z} are each hydrogen;
   and
   —X₁—(R,R—CH(R^{x})CR^{y}(R^{z}))—; wherein X₁ is —NH—, R^{x} and R^{z} are methyl, and R^{y} is hydrogen;
v) m is 0;
w) R^{a} and R^{d} are independently hydrogen or C₁₋₆alkyl, wherein C₁₋₆alkyl is optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
x) R^{a} and R^{d} are independently hydrogen or C₁₋₃alkyl, wherein C₁₋₃alkyl is optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
y) R^{a} and R^{d} are independently hydrogen, methyl or ethyl; or R^{a} and R^{c} are taken together with the atoms to which they are attached to form a 5-8 membered monocyclic ring optionally substituted with oxo;
z) R^{a} and R^{d} are independently hydrogen, methyl or ethyl;
aa) R^{b} is hydrogen, C₁₋₆alkyl, C₂₋₆alkoxycarbonyl, or cyano; or, R^{b} and R^{c} are taken together with the atoms to which they are attached to form a 5-8 membered monocyclic ring, optionally substituted with oxo;
bb) R^{b} is hydrogen or C₁₋₄alkyl; or, R^{b} and R^{c} are taken together with the atoms to which they are attached to form a 5-8 membered monocyclic ring, optionally substituted with oxo;
cc) R^{b} is hydrogen
dd) R^{c} is hydrogen, C₁₋₁₀alkyl, C₂₋₁₀alkenyl, C₃₋₇cycloalkyl, adamantyl, amino, arylcarbonyl, aryl, heteroaryl, or heterocyclyl; wherein C₁₋₁₀alkyl is optionally substituted with one to two substituents independently selected from the group consisting of C₁₋₄alkoxy, trifluoromethyl, aryl, heteroaryl, and heterocyclyl; and wherein any aryl- or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, nitro, methylthio, hydroxy, and cyano; or, R^{c} and R^{d} are taken together with the atoms to which they are attached to form a 5-8 membered monocyclic ring that optionally includes 1 to 2 O or S heteroatoms within the ring, and said ring is optionally substituted with oxo;
ee) R^{c} is hydrogen, C₁₋₆alkyl, C₂₋₆alkenyl, C₃₋₇cycloalkyl, adamantyl, heterocyclyl, arylcarbonyl, phenyl, or heteroaryl; wherein C₁₋₆alkyl is optionally substituted with one to two substituents independently selected from the group consisting of C₁₋₃alkoxy, trifluoromethyl, phenyl, heteroaryl, and heterocyclyl; and wherein any aryl-, phenyl-, or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, nitro, methylthio, hydroxy, and cyano; or, R^{c} and R^{d} are taken together with the atoms to which they are attached to form a 5-8 membered monocyclic ring and said ring is optionally substituted with oxo;
ff) R^{c} is hydrogen, C₁₋₆alkyl, C₂₋₆alkenyl, C₃₋₇cycloalkyl, heterocyclyl, phenylcarbonyl, phenyl, or heteroaryl; wherein C₁₋₆alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring;
gg) R^{c} is hydrogen, C₁₋₄alkyl, C₂₋₄alkenyl, cyclohexyl, phenylcarbonyl, phenyl, pyrimidinyl, furanyl, benzo[1,3]dioxolyl, or pyridinyl; wherein C₁₋₄alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring;
hh) R^{c} is hydrogen, C₁₋₄alkyl, C₂₋₄alkenyl, cyclohexyl, phenylcarbonyl, phenyl, pyrimidinyl, furanyl, benzo[1,3]dioxolyl, or pyridinyl; wherein C₁₋₄alkyl is optionally substituted with one to two substituents independently selected from the group consisting of methoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₃alkyl, C₁₋₃alkoxy, chloro, fluoro, bromo, trifluoromethoxy, nitro, hydroxy, and cyano; or, R^{c} and R^{d} are taken together with the atoms to which they are attached to form a 5-6 membered monocyclic ring;
   with the proviso that in any instance, only one ring optionally exists between R^{a} and R^{b}, R^{b} and R^{c}, or R^{c} and R^{d};
   and combinations of a) through hh) above.

In an embodiment, the present invention is directed to a method for treating, ameliorating, or preventing pain; comprising, consisting of, and/or consisting essentially of administering to a subject in need thereof, a therapeutically effective amount of a compound of Formula (Ia):

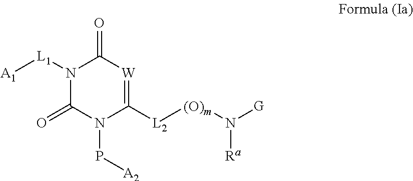

or an enantiomer, diastereomer, solvate, or pharmaceutically acceptable salt thereof;

wherein:
A₁ is hydrogen; aryl; heteroaryl; C₅₋₈cycloalkyl; or heterocyclyl provided that A₁ is other than piperidin-4-yl, N-t-butoxycarbonyl-piperidin-4-yl, or N-methyl-piperidin-3-yl; and wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, cyano, hydroxy, aminocarbonyl, C₁₋₆alkylaminocarbonyl, di(C₁₋₆alkyl)aminocarbonyl, and C₁₋₆alkylcarbonyl;
L₁ is —(CH₂)ᵣ— optionally substituted with one to three substituents independently selected from the group consisting of C₁₋₆alkyl, C₂₋₆alkenyl, C₂₋₆alkynyl, and halogen; provided that when A₁ is hydrogen, r is greater than or equal to 4;
r is an integer of 1 to 5;
P is —(CH₂)₄₋₆— when A₂ is hydrogen; and P is —(CH₂)₁₋₂— or —CH₂CH═CH—when A₂ is other than hydrogen;
A₂ is hydrogen, heteroaryl other than unsubstituted pyridin-2-yl, C₃₋₈cycloalkyl, or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, cyano, hydroxy, nitro, aminocarbonyl, C₁₋₆alkylcarbonylamino, and a non fused C₃₋₆cycloalkyloxy; wherein heteroaryl other than unsubstituted pyridin-2-yl and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, cyano, hydroxy, nitro, aminocarbonyl, C₁₋₆alkylcarbonylamino, and a non fused C₃₋₆cycloalkyloxy;
   provided that no more than two substituents on A₂ are aryl(C₁₋₆)alkoxy, phenyl, or a non fused C₃₋₆cycloalkyloxy;
   provided that when A₁ is unsubstituted phenyl and L₂ is —X₁—CH(R^{x})—(CR^{y}R^{z})— wherein X₁ is NH, and R^{x}, R^{y}, and R^{z} are each hydrogen, A₂ is other than unsubstituted phenyl; phenyl substituted with aryl(C₁₋₆)alkoxy or phenyl; or phenyl substituted at the meta position with cyano;
   and, further provided that when A₁ is unsubstituted phenyl and L₂ is —X₁(CH₂)₃— wherein X₁ is NH, A₂ is other than phenyl substituted with methoxy;
   and, provided that when A₁ is 3,4-dichloro-phenyl and P is —CH₂—, A₂ is other than phenyl substituted in the meta position with trifluoromethyl or trifluoromethoxy;
   and, further provided that when A₁ is 3,4-dichloro-phenyl and P is —(CH₂)₂—, A₂ is other than 4-methoxy-phenyl;
W is N or CH;
L₂ is a bivalent radical selected from the group consisting of
   —NH—C₅₋₇cycloalkyl-(CH₂)₀₋₂—; provided that when C₅₋₇cycloalkyl is cyclohexyl, Q is attached at either the 2- or cis-4-position relative to the position of —NH—;
   —X₂—(CH₂)₀₋₄—;
   —X₁—(CH₂)₂₋₃—X₃—(CH₂)₂₋₃—;
   —NH(CH₂)₁₋₄C(═O)— provided that at least one of R^{b}, R^{c}, or R^{d} is other than hydrogen and m is 0;
   —NHC(═O)—(CH₂)₁₋₄—;
   —C(═O)NH(CR^{y}R^{z})₂₋₅—;
   and
   —X₁—CH(R^{x})—(CR^{y}R^{z})₁₋₅—; such that when X₁ is a direct bond and W is C(R_{w}), then R^{x} of CH(R^{x}) is hydrogen;
   wherein X₁ is —NH—, O, S, or a direct bond; such that X₁ is other than O when W is N;
   X₂ is —CH═CH—;
   X₃ is O, S, NH, or C═O;
   R^{x}, R^{y}, and R^{z} are independently H or C₁₋₄alkyl;
   and provided that L₂ in any instance does not exceed 7 atoms in length;
   and further provided that when L₂ is —X₂—(CH₂)₀₋₄— or —C(═O)NH(CR^{y}R^{z})₂₋₅—, then R_{W} is hydrogen;
m is 0 or 1;
G is —C(═NR^{b})NR^{c}R^{d};
R^{a} and R^{d} are independently hydrogen or C₁₋₆alkyl, wherein C₁₋₆alkyl is optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
R^{b} is hydrogen, C₁₋₆alkyl, C₂₋₆alkoxycarbonyl, or cyano; or, R^{b} and R^{c} are taken together with the atoms to which they are attached to form a 5-8 membered monocyclic ring optionally substituted with oxo;
R^{c} is hydrogen, C₁₋₁₀alkyl, C₂₋₁₀alkenyl, C₃₋₇cycloalkyl, adamantyl, amino, arylcarbonyl, aryl, heteroaryl, or heterocyclyl; wherein C₁₋₁₀alkyl is optionally substituted with one to two substituents independently selected from the group consisting of C₁₋₄alkoxy, trifluoromethyl, aryl, heteroaryl, and heterocyclyl; and wherein any aryl- or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, nitro, methylthio, hydroxy, and cyano; or, R^{c} and R^{d} are taken together with the atoms to which they are attached to form a 5-8 membered monocyclic ring that optionally includes 1 to 2 O or S heteroatoms within the ring, and said ring is optionally substituted with oxo;
with the proviso that in any instance, only one ring optionally exists between R^{a} and R^{b}, R^{b} and R^{c}, or R^{c} and R^{d}.

In a further embodiment, the present invention is directed to a method for treating, ameliorating, or preventing pain; comprising, consisting of, and/or consisting essentially of administering to a subject in need thereof, a therapeutically effective amount of a compound of Formula (Ia):

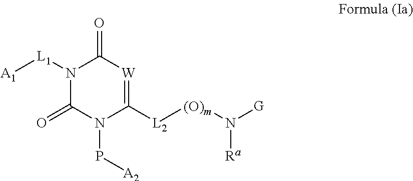

or an enantiomer, diastereomer, solvate, or pharmaceutically acceptable salt thereof;

wherein:
A₁ is hydrogen; aryl; heteroaryl; C₅₋₈cycloalkyl; or heterocyclyl other than piperidinyl; wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, cyano, hydroxy, aminocarbonyl, C₁₋₆alkylaminocarbonyl, di(C₁₋₆alkyl)aminocarbonyl, and C₁₋₆alkylcarbonyl;
L₁ is —(CH₂)ᵣ— optionally substituted with a substituent selected from the group consisting of C₁₋₄alkyl, C₂₋₄alkenyl, and C₂₋₄alkynyl; provided that r is 1 to 3 when A₁ is other than hydrogen; or r is 4 or 5 when A₁ is hydrogen;
P is —CH₂—;
A₂ is furanyl, pyridin-3-yl, pyridin-4-yl, or phenyl optionally substituted at the meta and para positions with one to three substituents independently selected from the group consisting of C₁₋₄alkyl, C₁₋₄alkoxy, halogen, halogenated C₁₋₃alkoxy, C₁₋₃alkylthio, hydroxy, amino, aminocarbonyl, C₁₋₃alkylcarbonylamino, and a non fused C₃₋₆cycloalkyloxy; and wherein furanyl, pyridin-3-yl, and pyridin-4-yl are optionally substituted with one to three substituents independently selected from the group consisting of C₁₋₄alkyl, C₁₋₄alkoxy, halogen, halogenated C₁₋₃alkoxy, C₁₋₃alkylthio, hydroxy, amino, aminocarbonyl, C₁₋₃alkylcarbonylamino, and a non fused C₃₋₆cycloalkyloxy;
provided that no more than two substituents on A₂ are non fused C₃₋₆cycloalkyloxy;
provided that when A₁ is unsubstituted phenyl and L₂ is —X₁—CH(R^{x})—(CR^{y}R^{z})— wherein X₁ is NH, and R^{x}, R^{y}, and R^{z} are each hydrogen, A₂ is other than unsubstituted phenyl;
and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
and, provided that when A₁ is 3,4-dichloro-phenyl, A₂ is other than phenyl substituted in the meta position with trifluoromethoxy;
W is N or CH;
L₂ is a bivalent radical selected from the group consisting of
   —NH—C₅₋₆cycloalkyl-(CH₂)₀₋₂—; provided that when C₅₋₆cycloalkyl is cyclohexyl, Q is attached at either the 2- or cis-4-position relative to the position of —NH—;
   —X₁—CH(R^{x})—(CR^{y}R^{z})₁₋₅—, wherein X₁ is —NH—, O, or S; and R^{x}, R^{y}, and R^{z} are each hydrogen; such that X₁ is other than O when W is N;
   —C(═O)NH(CH₂)₂—;
   and
   —X₁—(R,R—CH(R^{x})CR^{y}(R^{z}))—; wherein X₁ is —NH—, and R^{x} and R^{z} are methyl, and R^{y} is hydrogen;
   provided that when L₂ is —C(═O)NH(CH₂)₂—, then R_{W} is hydrogen;
m is 0 or 1;
G is —C(═NR^{b})NR^{c}R^{d};
R^{a} and R^{d} are independently hydrogen or C₁₋₃alkyl, wherein C₁₋₃alkyl is optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
R^{b} is hydrogen or C₁₋₄alkyl; or, R^{b} and R^{c} are taken together with the atoms to which they are attached to form a 5-8 membered monocyclic ring, optionally substituted with oxo;
R^{c} is hydrogen, C₁₋₆alkyl, C₂₋₆alkenyl, C₃₋₇cycloalkyl, adamantyl, heterocyclyl, arylcarbonyl, phenyl, or heteroaryl; wherein C₁₋₆alkyl is optionally substituted with one to two substituents independently selected from the group consisting of C₁₋₃alkoxy, trifluoromethyl, phenyl, heteroaryl, and heterocyclyl; and wherein any aryl-, phenyl-, or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, nitro, methylthio, hydroxy, and cyano; or, R^{c} and R^{d} are taken together with the atoms to which they are attached to form a 5-8 membered monocyclic ring and said ring is optionally substituted with oxo;
with the proviso that in any instance, only one ring optionally exists between R^{a} and R^{b}, R^{b} and R^{c}, or R^{c} and R^{d}.

A further embodiment of the present invention is directed to a method for treating, ameliorating, or preventing pain; comprising, consisting of, and/or consisting essentially of administering to a subject in need thereof, a therapeutically effective amount of a compound of Formula (Ia):

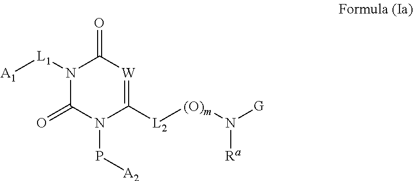

or an enantiomer, diastereomer, solvate, or pharmaceutically acceptable salt thereof;

wherein:
A₁ is substituted phenyl, benzofuranyl, thiazolyl, or thiophenyl; wherein phenyl is substituted with, and benzofuranyl, thiazolyl, and thiophenyl are optionally substituted with, one to two substituents independently selected from the group consisting of C₁₋₄alkyl, C₁₋₄alkoxy, halogen, nitro, halogenated C₁₋₄alkyl, halogenated C₁₋₄alkoxy, methylthio, amino, cyano, and C₁₋₄alkylcarbonyl;
L₁ is —(CH₂)ᵣ— optionally substituted with a substituent selected from the group consisting of methyl and allyl, and r is 1 to 3;
A₂ is pyridin-3-yl, pyridin-4-yl, or phenyl optionally substituted at the meta and para positions with one to two substituents independently selected from the group consisting of methyl, ethyl, methoxy, ethoxy, isopropyloxy, trifluoromethoxy, difluoromethoxy, hydroxy, aminocarbonyl, and methylcarbonylamino; wherein pyridin-3-yl and pyridin-4-yl are optionally substituted with one to two substituents independently selected from the group consisting of methyl, ethyl, methoxy, ethoxy, isopropyloxy, trifluoromethoxy, difluoromethoxy, hydroxy, aminocarbonyl, and methylcarbonylamino;
P is —CH₂—;
W is N or CH;
L₂ is a bivalent radical selected from the group consisting of
   —NH-cyclohexyl-(CH₂)₀₋₂— and Q is attached at either the 2- or cis-4-position relative to the position of —NH—;
   —X₁—CH(R^{x})—(CR^{y}R^{z})₁₋₅—; wherein X₁ is —NH— or S; and R^{x}, R^{y}, and R^{z} are each hydrogen;
   and
   —X₁—(R,R—CH(R^{x})CR^{y}(R^{z}))—; wherein X₁ is —NH—, and R^{x} and R^{z} are methyl, and R^{y} is hydrogen;
m is 0;
G is —C(═NR^{b})NR^{c}R^{d};
R^{a} and R^{d} are independently hydrogen, methyl or ethyl; or R^{a} and R^{c} are taken together with the atoms to which they are attached to form a 5-8 membered monocyclic ring optionally substituted with oxo;
R^{b} is hydrogen;
R^{c} is hydrogen, C₁₋₆alkyl, C₂₋₆alkenyl, C₃₋₇cycloalkyl, heterocyclyl, phenylcarbonyl, phenyl, or heteroaryl; wherein C₁₋₆alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring;
with the proviso that in any instance, only one ring optionally exists between R^{a} and R^{b}, R^{b} and R^{c}, or R^{c} and R^{d}.

A further embodiment of the present invention is directed to a method for treating, ameliorating, or preventing pain; comprising, consisting of, and/or consisting essentially of administering to a subject in need thereof, a therapeutically effective amount of a compound of Formula (Ia):

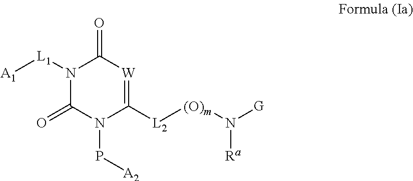

or an enantiomer, diastereomer, solvate, or pharmaceutically acceptable salt thereof;

wherein:
A₁ is phenyl or benzofuranyl; wherein phenyl is substituted at either the 4-position or 3 and 4-positions with one to two substituents independently selected from the group consisting of ethyl, methoxy, fluoro, chloro, nitro, difluoromethoxy, and methylthio;
L₁ is —CH₂— optionally substituted with methyl or allyl;
A₂ is phenyl substituted at the para position with a substituent selected from the group consisting of methoxy, ethoxy, isopropyloxy, difluoromethoxy, hydroxy, and aminocarbonyl; or A₂ is pyridin-3-yl or pyridin-4-yl substituted with methoxy;
P is —CH₂—;
W is N or CH;
L₂ is a bivalent radical selected from the group consisting of
   —NH-cyclohexyl-(CH₂)₀₋₂— and Q is attached at either the 2- or cis-4-position relative to the position of —NH—;
   —X₁—CH(R^{x})—(CR^{y}R^{z})—; wherein X₁ is —NH— or S and R^{x}, R^{y}, and R^{z} are each hydrogen;
   and
   —X₁—(R,R—CH(R^{x})CR^{y}(R^{z}))—; wherein X₁ is —NH—, R^{x} and R^{z} are methyl, and R^{y} is hydrogen;
m is 0;
G is —C(═NR^{b})NR^{c}R^{d};
R^{a} and R^{d} are independently hydrogen, methyl or ethyl;
R^{b} is hydrogen;
R^{c} is hydrogen, C₁₋₄alkyl, C₂₋₄alkenyl, cyclohexyl, phenylcarbonyl, phenyl, pyrimidinyl, furanyl, benzo[1,3]dioxolyl, or pyridinyl; wherein C₁₋₄alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring
with the proviso that in any instance, only one ring optionally exists between R^{a} and R^{b}, R^{b} and R^{c}, or R^{c} and R^{d}.

A further embodiment of the present invention is directed to a method for treating, ameliorating, or preventing pain; comprising, consisting of, and/or consisting essentially of administering to a subject in need thereof, a therapeutically effective amount of a compound of Formula (I):

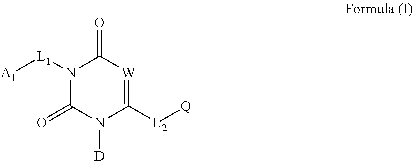

selected from the group consisting of
a compound wherein A₁ is phenyl, L₁ is —CH₂—, D is —CH₂-(4-fluoro-phenyl), W is N, L₂ is —NH(CH₂)₂—, and Q is —NHC(═NH)NH₂;
a compound wherein A₁ is phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is phenyl, L₁ is —CH₂—, D is —CH₂-(4-methylcarboxy-phenyl), W is N, L₂ is —NH(CH₂)₂—, and Q is —NHC(═NH)NH₂;
a compound wherein A₁ is phenyl, L₁ is —(CH₂)₂—, D is —CH₂-(4-methoxy-phenyl), W is N, L₂ is —NH(CH₂)₂—, and Q is —NHC(═NH)NH₂;
a compound wherein A₁ is H, L₁ is —(CH₂)₄—, D is —CH₂-(4-methoxy-phenyl), W is N, L₂ is —NH(CH₂)₂—, and Q is —NHC(═NH)NH₂;
a compound wherein A₁ is furan-2-yl, L₁ is —CH₂—, D is —CH₂-(4-methoxy-phenyl), W is N, L₂ is —NH(CH₂)₂—, and Q is —NHC(═NH)NH₂;
a compound wherein A₁ is phenyl, L₁ is —CH₂—, D is —CH₂-(3-trifluoromethyl-phenyl), W is N, L₂ is —NH(CH₂)₂—, and Q is —NHC(═NH)NH₂;
a compound wherein A₁ is phenyl, L₁ is —CH₂—, D is —CH₂-(4-t-butyl-phenyl), W is N, L₂ is —NH(CH₂)₂—, and Q is —NHC(═NH)NH₂;
a compound wherein A₁ is phenyl, L₁ is —CH₂—, D is —CH₂-(4-nitro-phenyl), W is N, L₂ is —NH(CH₂)₂—, and Q is —NHC(═NH)NH₂;
a compound wherein A₁ is phenyl, L₁ is —CH₂—, D is —CH₂-(4-methoxy-phenyl), W is N, L₂ is —NH(CH₂)₂—, and Q is —ONHC(═NH)NH₂;
a compound wherein A₁ is phenyl, L₁ is —CH₂—, D is —CH₂-pyridin-4-yl, W is N, L₂ is —NH(CH₂)₂—, and Q is —NHC(═NH)NH₂;
a compound wherein A₁ is phenyl, L₁ is —CH₂—, D is —CH₂-(4-ethoxy-phenyl), W is N, L₂ is —NH(CH₂)₂—, and Q is —NHC(═NH)NH₂;
a compound wherein A₁ is phenyl, L₁ is —CH₂—, D is —CH₂-(4-difluoromethoxy-phenyl), W is N, L₂ is —NH(CH₂)₂—, and Q is —NHC(═NH)NH₂;
a compound wherein A₁ is phenyl, L₁ is —CH₂—, D is —CH₂-(4-n-butyl-phenyl), W is N, L₂ is —NH(CH₂)₂—, and Q is —NHC(═NH)NH₂;
a compound wherein A₁ is phenyl, L₁ is —CH₂—, D is —CH₂-(4-trifluoromethyl-phenyl), W is N, L₂ is —NH(CH₂)₂—, and Q is —NHC(═NH)NH₂;
a compound wherein A₁ is 2-fluoro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is phenyl, L₁ is —CH₂—, D is —CH₂-(4-trifluoromethoxy-phenyl), W is N, L₂ is —NH(CH₂)₂—, and Q is —NHC(═NH)NH₂;
a compound wherein A₁ is 3-methoxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 2-methoxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is phenyl, L₁ is —CH₂—, D is —CH₂-(4-aminocarbonyl-phenyl), W is N, L₂ is —NH(CH₂)₂—, and Q is —NHC(═NH)NH₂;
a compound wherein A₁ is phenyl, L₁ is —CH₂—, D is —CH₂-(4-methylcarboxylamino-phenyl), W is N, L₂ is —NH(CH₂)₂—, and Q is —NHC(═NH)NH₂;
a compound wherein A₁ is 4-fluoro-phenyl, L₁ is —CH₂—, D is —CH₂-(4-ethoxy-phenyl), W is N, L₂ is —NH(CH₂)₂—, and Q is —NHC(═NH)NH₂;
a compound wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —ONHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═N—CN)NH₂;
a compound wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH₂;
a compound wherein A₁ is 4-chloro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-methoxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₄—, and Q is —NHC(═NH)NH₂;
a compound wherein A₁ is 4-fluoro-phenyl, L₁ is —CH₂—, D is —(CH₂)₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-n-propyl-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-i-propyl-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-cyclopentyloxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methylthio-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-ethyl-phenyl), W is N, L₂ is —NH(CH₂)₂—, and Q is —NHC(═NH)NH₂;
a compound wherein A₁ is 3-chloro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-trifluoromethoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-difluoromethoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is cis-racemic-1,2-cyclohexyl, and Q is —NHC(═NH)NH₂;
a compound wherein A₁ is 3,4-dichloro-phenyl, L₁ is —CH₂—, D is —CH₂-(4-methoxy-phenyl), W is N, L₂ is trans (1S,2S)-cyclohexyl-, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-methylthio-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-ethyl-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is trans(1R,2R)-cyclohexyl-, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NH(3,5-dihydro-imidazol-4-on-2-yl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NH(4,5-dihydro-1H-imidazol-2-yl);
a compound wherein A₁ is 3,4-dichloro-phenyl, L₁ is —CH₂—, D is —CH₂-(4-methylcarbonylamino-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-aminocarbonyl-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(3-ethoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH-ethyl;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH-propyl;
a compound wherein A₁ is 3,4-dichloro-phenyl, L₁ is —CH₂—, D is —CH₂-(4-methoxy-phenyl), W is N, L₂ is pyrrolindin-1-yl, and Q is 3—NHC(═NH)NH₂;
a compound wherein A₁ is 4-chloro-phenyl, L₁ is —CH₂—, D is —CH₂-(4-methoxy-phenyl), W is N, L₂ is -trans (1R,2R)-cyclohexyl-, and Q is —NHC(═NH)NH₂;
a compound wherein A₁ is 3,4-dichloro-phenyl, L₁ is —CH₂—, D is —CH₂-(3-difluoromethoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(i-propyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —N(ethyl)C(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is 2-imino-imidazolid-1-yl;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(n-butyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(cyclohexyl);
a compound wherein A₁ is 3,4-dichloro-phenyl, L₁ is —CH₂—, D is —CH₂-(4-methoxy-phenyl) W is N, L₂ is —NH(CH₂)₂—, and Q is —NHC(═NH)NH(benzyl);
a compound wherein A₁ is 3,4-dichloro-phenyl, L₁ is —CH₂—, D is —CH₂-(4-methoxy-phenyl), W is N, ¹₂ is —NH(CH₂)₂, and Q is —NHC(═NH)NH(tetrahydrofuran-2-ylmethyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenylethyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(furan-2-ylmethyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(2-methoxy-ethyl);
a compound wherein A₁ is 3,4-dichloro-phenyl, L₁ is —CH₂—, D is —CH₂-(4-methoxy-phenyl), W is N, L₂ is —NH(CH₂)₃—, and Q is —NHC(═NH)NH₂;
a compound wherein A₁ is 3,4-dichloro-phenyl, L₁ is —CH₂—, D is —(CH₂)₆—H, W is N, L₂ is —NH(CH₂)₃—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(allyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methoxy-phenyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-chloro-phenyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-trifluoromethyl-phenyl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(pyridin-3-yl);
a compound wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methylcarbonyl-phenyl);
a compound wherein A₁ is furan-3-yl, L₁ is —CH₂—, D is —CH₂-(4-methoxy-phenyl), W is N, L₂ is —NH(CH₂)₂—, and Q is —NHC(═NH)NH₂;
a compound wherein A₁ is thiophen-2-yl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-methoxy-phenyl, L₁ is an R,S-mixture of —CH(CH₃)—, D is —CH₂-(4-methoxy-phenyl), W is N, L₂ is —NH(CH₂)₂—, and Q is —NHC(═NH)NH₂;
a compound wherein A₁ is 4-difluoromethoxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is phenyl, L₁ is —CH₂—, D is —CH₂-(4-methoxy-phenyl), W is

CH, L₂ is —NH(CH₂)₂—, and Q is —NHC(═NH)NH₂;
4-methoxy-phenyl, L₁ is an R,S-mixture of —CH(allyl)-, D is —CH₂-(4-methoxy-phenyl), W is N, L₂ is —NH(CH₂)₂—, and Q is —NHC(═NH)NH₂;
a compound wherein A₁ is 4-chloro-phenyl, L₁ is an R,S-mixture of —CH(allyl)-, D is —CH₂-(4-methoxy-phenyl), W is N, L₂ is —NH(CH₂)₂—, and Q is —NHC(═NH)NH₂;
a compound wherein A₁ is 4-methoxy-phenyl, L₁ is —CH₂—, D is —CH₂-(4-methoxy-phenyl), W is CH, L₂ is —NH(CH₂)₂—, and Q is —NHC(═NH)NH₂;
a compound wherein A₁ is 4-methoxy-phenyl, L₁ is —CH₂—, D is —CH₂-(6-methoxy-pyridin-3-yl), W is N, L₂ is —NH(CH₂)₂—, and Q is —NHC(═NH)NH₂;
a compound wherein A₁ is 4-methoxy-phenyl, L₁ is —CH₂—, D is —CH₂-(4-methoxy-cyclohexyl), W is N, L₂ is —NH(CH₂)₂—, and Q is —NHC(═NH)NH₂;
a compound wherein A₁ is 4-fluoro-phenyl, L₁ is —CH₂—, D is —CH₂-(4-nitro-phenyl), W is N, L₂ is —NH(CH₂)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-(morpholin-4-yl)-eth-1-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(3-(morpholin-4-yl)-prop-1-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-cyano-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-nitro-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(1,3-benzodioxol-5-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHNH₂;
a compound wherein A₁ is 3-nitro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-nitro-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3-amino-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-cyano-phenyl, L₁ is —CH₂—, D is —CH₂-(4-methoxy-phenyl), W is N, L₂ is —NH(CH₂)₂—, and Q is —NHC(═NH)NH₂;
a compound wherein 3-cyano-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is4-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-carboxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, ¹₂ is —NH(CH₂)C(Me)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-bromo-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl-ethyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-ethoxycarbonyl-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2,4-difluoro-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-decanyl);
a compound wherein A₁ is 4-t-butoxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-hydroxy-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 2-chloro-thiazol-4-yl, L₁ is —CH₂—, D is —CH₂-(4-methoxy-phenyl), W is N, L₂ is —NH(CH₂)₂—, and Q is —NHC(═NH)NH₂;
a compound wherein A₁ is benzofuran-2-yl, L₁ is —CH₂—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxy-phenyl), W is N, L₂ is —NH(CH₂)₂—, and Q is —N(Me)C(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(CH₂CF₃);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(3-methoxypropyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)piperidin-1-yl;
4-fluoro-phenyl, L₁ is —CH₂—, D is —CH₂-(4-methoxy-phenyl), W is N, L₂ is —NH(CH₂)₂—, and Q is —NHC(═NH)N(Me)phenyl;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-fluoro-phenyl);
a compound wherein A₁ is 4-fluoro-phenyl, L₁ is —CH₂—, D is —CH₂-(4-methoxy-phenyl), W is N, L₂ is ⁻NH(CH₂)₂—, and Q is —NHC(═NH)NH(4-fluoro-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-methyl-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(t-butyl);
a compound wherein A₁ is 4-chloro-phenyl, L₁ is —CH₂—, D is —CH₂-(4-amino-phenyl), W is N, L₂ is —NH(CH₂)₂—, and Q is —NHC(═NH)NH₂;
a compound wherein A₁ is t-butyl, L₁ is —CH₂—, D is —CH₂-(4-methoxy-phenyl), W is N, L₂ is —NH(CH₂)₂—, and Q is —NHC(═NH)NH₂;
a compound wherein A₁ is cyclopentyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-amino-phenyl, L₁ is —CH₂—, D is —CH₂-(4-methoxy-phenyl), W is N, L₂ is —NH(CH₂)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(adamantan-2-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-trifluoromethoxy-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-hydroxy-phenyl);
a compound wherein A₁ is 4-chloro-phenyl, L₁ is —CH₂—, D is —CH₂-phenyl, W is N, L₂ is —NH(CH₂)₂—, and Q is —NHC(═NH)NH₂;
a compound wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is 1,4-cyclohexyl, and Q is —NHC(═NH)NH₂;
a compound wherein A₁ is 4-fluoro-phenyl, L₁ is —CH₂—, D is —CH₂-(4-methoxy-phenyl), W is N, L₂ is —NHCH₂C(═O)—, and Q is —NHC(═NC(═O)O-t-butyl)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-methylthio-phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(C(═O)phenyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyrimidin-2-yl);
a compound wherein A₁ is 4-fluoro-phenyl, L₁ is —CH₂—, D is —CH₂-(4-methoxy-phenyl), W is N, L₂ is —NH((S)—CHMe)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH((R)—CHMe)₂—, and Q is —NHC(═NH)NH_{2′};
a compound wherein A₁ is 4-fluoro-phenyl, L₁ is —CH₂—, D is —CH₂-(4-methoxy-phenyl), W is N, L₂ is —NH(CH₂)₂—, and Q is —NH(═NH)NH(4-trifluoromethyl-5,6,7,8-tetrahydro-quinazolin-2-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(5-methyl-pyridin-2-yl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)morpholin-4-yl;
a compound wherein A₁ is 4-chloro-phenyl, L₁ is —CH₂—, D is —CH₂-furan-2-yl, W is N, L₂ is —NH(CH₂)₂—, and Q is —NHC(═NH)NH₂;
a compound wherein A₁ is 4-chloro-phenyl, L₁ is —CH₂—, D is —CH₂-(4-methoxy-phenyl), W is N, L₂ is —NH(CH₂)₅—, and Q is —NHC(═NH)NH₂;
a compound wherein A₁ is 4-methoxy-phenyl, L₁ is —CH₂—, D is —CH₂-(4-hydroxy-phenyl), W is N, L₂ is —NH(CH₂)₂—, and Q is —NHC(═NH)NH₂;
a compound wherein A₁ is 4-chloro-phenyl, L₁ is —CH₂—, D is —CH₂-(4-methoxy-phenyl), W is N, L₂ is —NH(CH₂)₆—, and Q is —NHC(═NH)NH₂;
a compound wherein A₁ is 4-methoxy-phenyl, L₁ is —(CH₂)₂—, D is —CH₂-(4-methoxy-phenyl), W is N, L₂ is —NH(CH₂)₂—, and Q is —NHC(═NH)NH₂;
a compound wherein A₁ is 4-methoxy-phenyl, L₁ is —(CH₂)₃—, D is —CH₂-(4-methoxy-phenyl), W is N, L₂ is —NH(CH₂)₂—, and Q is —NHC(═NH)NH₂;
a compound wherein A₁ is 3,4-dichloro-phenyl, L₁ is —CH₂—, D is —CH₂-(4-methoxycarbonyl-phenyl), W is N, L₂ is —NH(CH₂)₂—, and Q is —NHC(═NH)NH₂;
a compound wherein A₁ is phenyl, L₁ is —CH₂—, D is —CH₂-(4-n-butyloxy-phenyl), W is N, L₂ is —NH(CH₂)₂—, and Q is —NHC(═NH)NH₂;
a compound wherein A₁ is 4-chloro-phenyl, L₁ is —CH₂—, D is —CH₂-phenyl, W is N, L₂ is —NH(CH₂)₂—, and Q is —NHC(═NH)NH₂;
a compound wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHC(═O)methyl;
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(allyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(i-propyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-propyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(ethyl);
a compound wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(methyl);
a compound wherein A₁ is 4-methoxy-phenyl, L₁ is —CH₂—, D is —CH₂-(4-methoxy-phenyl), W is CH, L₂ is —C(═O)NH(CH₂)₂—, and Q is —NHC(═NH)NH₂;
a compound wherein A₁ is 4-methoxy-phenyl, L₁ is —CH₂—, D is —CH₂-(4-methoxy-phenyl), W is CH, L₂ is —O(CH₂)₂—, and Q is —NHC(═NH)NH₂;
a compound wherein A₁ is 4-methoxy-phenyl, L₁ is —CH₂—, D is —CH₂-(4-methoxy-phenyl), W is CH, L₂ is —S(CH₂)₂—, and Q is —NHC(═NH)NH₂;
a compound wherein A₁ is 4-methoxy-phenyl, L₁ is —CH₂—, D is —CH₂-(4-methoxy-phenyl), W is CH, L₂ is —(CH₂)₃—, and Q is —NHC(═NH)NH₂;
   and pharmaceutically acceptable salts thereof.

For use in medicine, salts of compounds of Formula (I) refer to non-toxic “pharmaceutically acceptable salts.” Other salts may, however, be useful in the preparation of compounds of Formula (I) or of their pharmaceutically acceptable salts thereof. Suitable pharmaceutically acceptable salts of compounds of Formula (I) include acid addition salts which can, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts, such as sodium or potassium salts; alkaline earth metal salts, such as calcium or magnesium salts; and salts formed with suitable organic ligands, such as quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases that may be used in the preparation of pharmaceutically acceptable salts include acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholin, piperazine, potassium hydroxide, 1-(2-hydroxyethyl)-pyrrolidine, secondary amine, sodium hydroxide, triethanolamine, tromethamine and zinc hydroxide.

Embodiments of the present invention include prodrugs of compounds of Formula (I). In general, such prodrugs will be functional derivatives of the compounds that are readily convertible in vivo into the required compound. Thus, in the methods of treating or preventing embodiments of the present invention, the term “administering” encompasses the treatment or prevention of the various diseases, conditions, syndromes and disorders described with the compound specifically disclosed or with a compound that may not be specifically disclosed, but which converts to the specified compound in vivo after administration to a patient. Conventional procedures for the selection and preparation of suitable prodrug derivatives are described, for example, in “Design of Prodrugs”, ed. H. Bundgaard, Elsevier, 1985.

Where the compounds according to embodiments of this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula I.

Where the processes for the preparation of the compounds according to certain embodiments of the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

One embodiment of the present invention is directed to a composition, including a pharmaceutical composition, comprising, consisting of, and/or consisting essentially of the (+)-enantiomer of a compound of Formula (I) wherein said composition is substantially free from the (−)-isomer of said compound. In the present context, substantially free means less than about 25%, preferably less than about 10%, more preferably less than about 5%, even more preferably less than about 2% and even more preferably less than about 1% of the (−)-isomer calculated as.

$$\% ( + ) - enantiomer = \frac{( mass ( + ) - enantiomer )}{( mass ( + ) - enantiomer ) + ( mass ( - ) - enantiomer )} \times 100.$$

Another embodiment of the present invention is a composition, including a pharmaceutical composition, comprising, consisting of, and consisting essentially of the (−)-enantiomer of a compound of Formula (I) wherein said composition is substantially free from the (+)-isomer of said compound. In the present context, substantially free from means less than about 25%, preferably less than about 10%, more preferably less than about 5%, even more preferably less than about 2% and even more preferably less than about 1% of the (+)-isomer calculated as

$$\% ( - ) - enantiomer = \frac{( mass ( - ) - enantiomer )}{( mass ( + ) - enantiomer ) + ( mass ( - ) - enantiomer )} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups, such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice. Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent

By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

Solid oral dosage forms, such as tablets or capsules, containing the compounds of the present invention may be administered in at least one dosage form at a time, as appropriate. It is also possible to administer the compounds in sustained release formulations.

Additional oral forms in which the present inventive compounds may be administered include exilirs, solutions, syrups, and suspensions; each optionally containing flavoring agents and coloring agents.

Alternatively, compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a white wax or white soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example intracavernosally, intravenously, intramuscularly, subcutaneously, intradermally or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus for liquid oral preparations, such as suspensions, syrups, elixirs and solutions, suitable carriers, excipients and diluents include water, glycols, oils, alcohols, flavoring agents, preservatives, stabilizers, coloring agents and the like; for solid oral preparations, such as powders, capsules and tablets, suitable carriers, excipients and diluents include starches, sugars, diluents, granulating agents, lubricants, binders, disintegrating agents and the like. Solid oral preparations also may be optionally coated with substances, such as, sugars, or be enterically -coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives, such as solubilizers and preservatives.

A therapeutically effective amount of a compound of Formula (I) or a pharmaceutical composition thereof includes a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about 4 times per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition is preferably provided in the form of tablets containing about 0.01, about 10, about 50, about 100, about 150, about 200, about 250, and about 500 milligrams of a compound of Formula (I).

Advantageously, a compound of Formula (I) may be administered in a single daily dose, or the total daily dosage may be administered in divided doses of two, three and four times daily.

Optimal dosages of a compound of Formula (I) to be administered may be readily determined and will vary with the particular compound used, the mode of administration, the strength of the preparation and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is required for a subject in need thereof.

As Prokineticin 1 receptor antagonists, the compounds of Formula (I) are useful in methods for treating, ameliorating, or preventing pain in a subject, including an animal, a mammal and a human. Such methods comprise, consist of and/or consist essentially of administering to a subject, including an animal, a mammal, and a human in need of such treatment or prevention a therapeutically effective amount of a compound, salt or solvate of Formula (I).

Abbreviations used in the instant specification, particularly the Schemes and Examples, are as follows:
Boc=tert-butoxycarbonyl
BuLi=n-butyllithium
Cpd or Cmpd=compound
d=day/days
DCM=dichloromethane
DIAD=diisopropyl azodicarboxylate
DIPEA or DIEA=diisopropylethylamine
DMEM=Dulbecco's Modified Eagle Medium
DMF=N,N-dimethylformamide
DMSO=dimethylsulfoxide
EDCI=1-(3-Dimethylaminopropyl)-3-ethylcarbodiimide hydrochloride
EtOAc=ethyl acetate
EtOH=ethanol
h=hour/hours
HBTU=O-Benzotriazol-1-yl-N,N,N′,N′-tetramethyluronium hexafluorophosphate
LDA=lithium diisopropyamide
M=molar
MeCN=acetonitrile
MeOH=methanol
min=minutes
NaOMe=sodium methoxide
PyBOP=benzotriazole-1-yl-oxy-tris-pyrrolidino-phosphonium hexafluorophosphate
rt/RT=room temperature
THF=tetrahydrofuran
TFA=trifluoroacetic acid

### General Schemes

Representative compounds of the present invention can be synthesized in accordance with the general synthetic methods described below and are illustrated in the schemes that follows. The starting materials and reagents used in the schemes that follow are understood to be either commercially available or prepared by methods known to those skilled in the art. Since the schemes are an illustration, the invention should not be construed as being limited by the chemical reactions and conditions expressed.

Scheme A illustrates the general synthesis of compounds of the present invention wherein L₂ is other than —NHC(═O)—(CH₂)₁₋₄—, —C(═O)NH(CR^{y}R^{z})₂₋₅—, and —X₂—(CH₂)₀₋₄—. In Scheme A, X₁ of L₂ is NH. A compound of formula A1 may be methylated with a methylating agent such as methyl iodide in a polar solvent such as methanol to give a compound of formula A2. A compound of formula A2 may be condensed with an appropriately substituted isocyanate such as N-chlorocarbonyl isocyanate in the presence of excess tertiary amine such as diisopropylethylamine to give a triazine of formula A3.

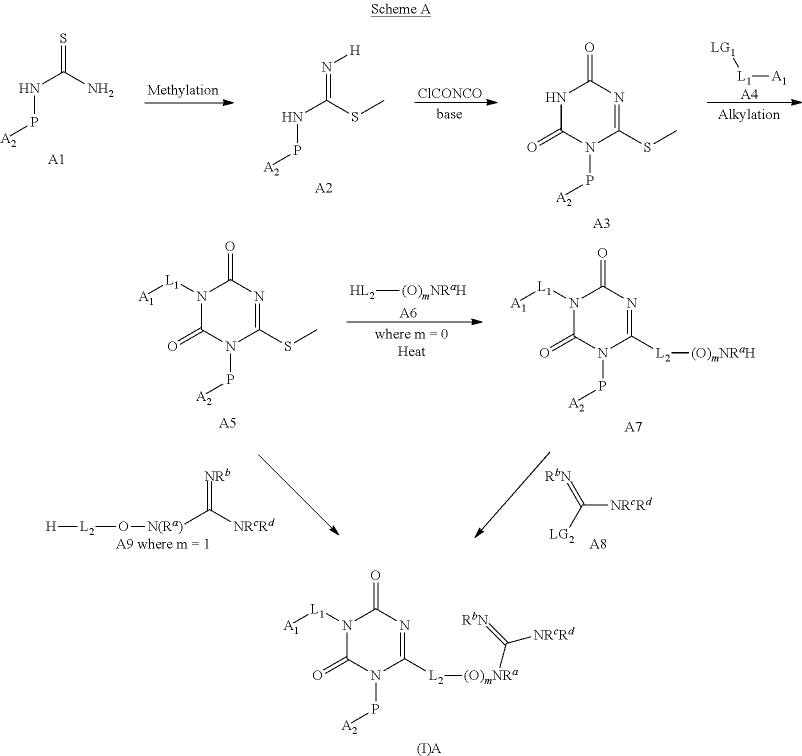

A compound of formula A3 may be alkylated with a compound of formula A4, wherein LG₁ is a leaving group, using conventional chemistry known to one versed in the art. For instance, when LG₁ is a hydroxy group, compound A4 may be coupled with compound A3 with the aid of a coupling agent such as DIAD in the presence of triphenylphosphine in a non-alcoholic polar solvent such as THF or methylene chloride. Alternatively, LG₁ may be a halide, tosylate, or the like such that LG₁ is displaced by the amino portion of a compound of A3 to give a compound of formula A5.

A compound of formula A5 may be further elaborated by nucleophilic substitution with a compound of formula A6 (wherein X₁ is NH and m is zero) to provide a compound of formula A7. One versed in the art will recognize that when L₂ is asymmetrical, a nitrogen-protecting group may be necessary to avoid competing reactions. A G-substituent of Formula (I) may be installed by treatment of the terminal amine of a compound of formula A7 with an activated amidine of formula A8 wherein LG₂ is a leaving group such as a halide, an alkoxide, an imidazole or pyrazole, an activated alkoxide, or the like, to give compound IA of Formula (I) wherein m is zero. Alternatively, when m is equal to one, an oxy-guanidine substituent may be incorporated by treatment of a compound of formula A7 with a compound of formula A9 to form a compound (I)A of Formula (I) wherein m is one.

Scheme B illustrates the general synthesis of compounds of the present invention wherein L₂ is —NHC(═O)—(CH₂)₁₋₄—. A compound of formula A5 may be converted to its corresponding amine by treatment with ammonia, or other source of ammonia such as ammonium hydroxide, to give a compound of formula B1. The amino group of a compound B1 may be acylated using conventional chemistry with a compound of formula B2, wherein LG₃ is a leaving group such as a halide when B2 is an acid chloride, a hydroxy group when B2 is a carboxylic acid, an alkylcarboxylate when B2 is an anhydride, or an imidazole when B2 is an acylimidazole. Alternatively, B2 may be an activated ester or the like. The K substituent of compounds of formula B2 is either a leaving group LG₁ as defined herein, or K is an R^{a}-substituted amino group protected with an appropriate amino-protecting group (PG).

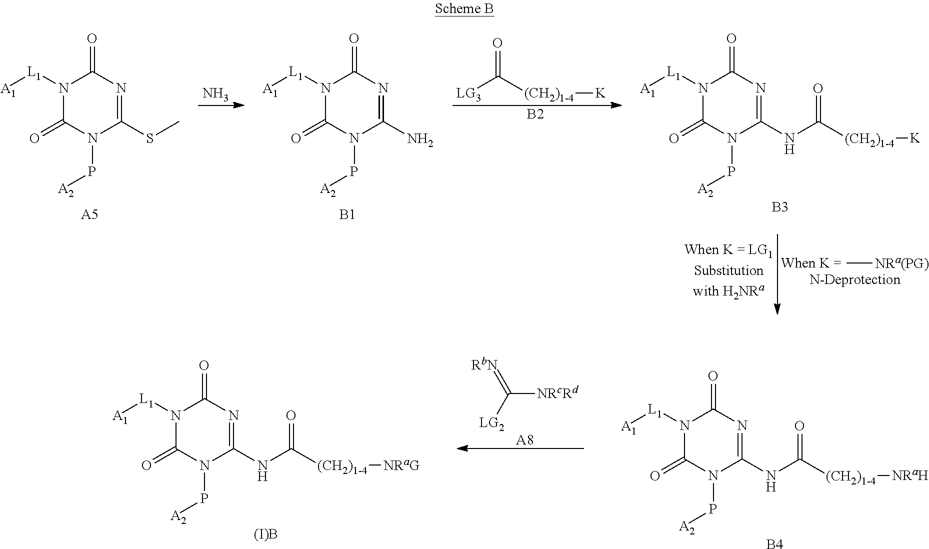

To prepare a compound of formula B4, a compound of formula B3 may either be N-deprotected (when K is —NR^{a}(PG)) using reagents and methods known to one versed in the art, or may undergo a nucleophilic displacement with amine H₂NR^{a} (when K is a LG₁). The resulting amine of formula B4 may then be treated with an activated amidine of formula A8 to give a compound (I)B of Formula (I).

Scheme C describes the general synthesis of compounds of the present invention wherein X₁ of L₂ is a direct bond and L₂ is any of those which contains X₁. A compound of formula C1 may be condensed with an isocyanate of formula C2 to give a compound of formula C3 which, upon heating, affords a triazine of formula C4. The amino group of a compound of formula C4 may be appropriately substituted using an alkylating agent of formula C5 to afford a compound of formula C6. A G-substituent may be introduced into a compound of formula C6 using the methods described herein to provide a compound (I)C of Formula (I).

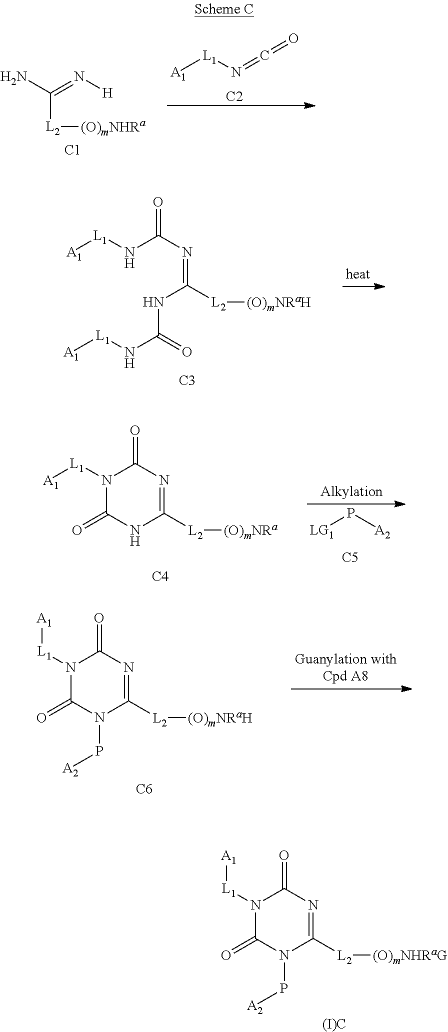

Scheme D illustrates the general synthesis of compounds of the present invention wherein W is C(R_{w}), L₂ is other than —NHC(═O)—(CH₂)₁₋₄— or —C(═O)NH(CR^{y}R^{z})₂₋₅—, and X₁ of L₂ is NH, O, or S. A compound of formula D1 may be condensed with a compound of formula D2 with heating (wherein LG₂ is C₁₋₄alkoxy, chloro, or the like) to form a compound of formula D3. A compound of formula D3 may then be treated with phosphorus oxychloride, PCl₅, or the like and heated to afford a compound of formula D4; alternatively, the bromo analog may be used in this synthetic sequence, which is prepared from D3 using phosphorus oxybromide in place of phosphorus oxychloride. A compound of formula C5 may be used to install —P-A₂ via conventional alkylation procedures. A compound of formula D5 may be elaborated via a nucleophilic displacement of the chloride or bromide with a compound of Formula D5a (wherein X₁ is NH, O, or S) to afford a compound of formula D6. Further elaboration using the chemistry described herein provides compound (I)D of Formula (I).

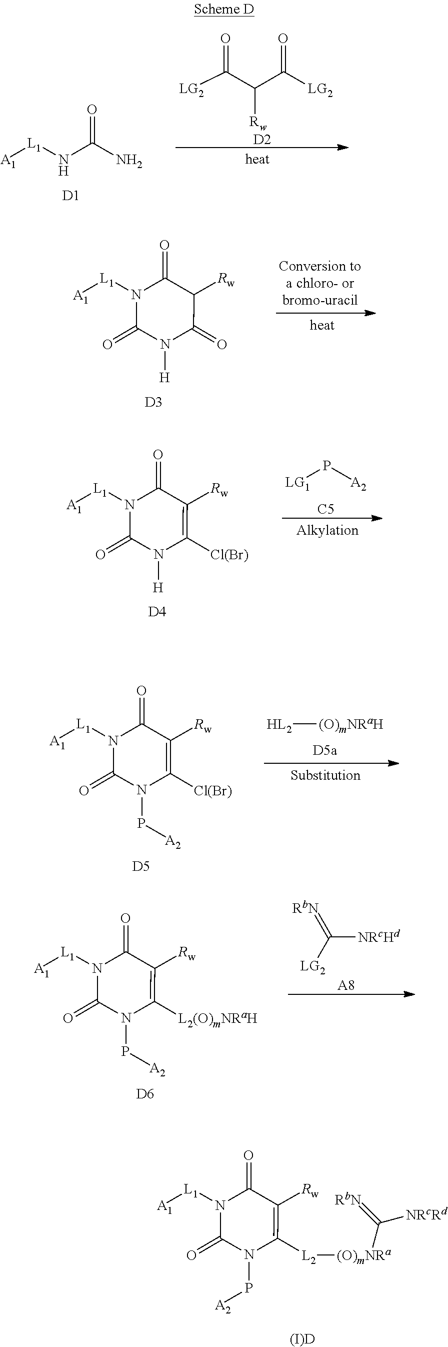

Scheme E illustrates the general synthesis of compounds of the present invention wherein W is C(R_{W}) and L₂ is —NHC(═O)—(CH₂)₁₋₄—. A compound of formula D5 may be treated with ammonia or other source of ammonia such as ammonium hydroxide to afford the corresponding amino compound of formula E1. The amino group may be acylated with a compound of formula B2 and further elaborated to a compound WE of Formula (I) using the methods described herein.

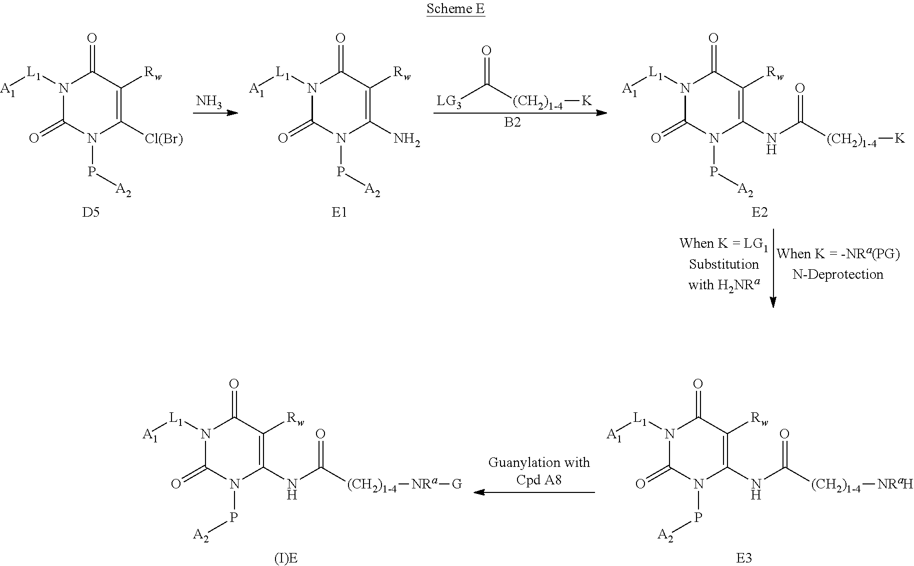

Scheme F illustrates the general synthesis of compounds of the present invention wherein W is C(R_{w}), X₁ of L₂ is a direct bond and L₂ is any one of those which includes X₁. A compound of formula F1 may be condensed with a compound of formula F2 under basic conditions in the presence of a C₁₋₄ alkyl alcohol to form a compound of formula F3. A compound of formula F3 may be condensed with a urea of formula F4 to form a cyclic compound of formula F5.

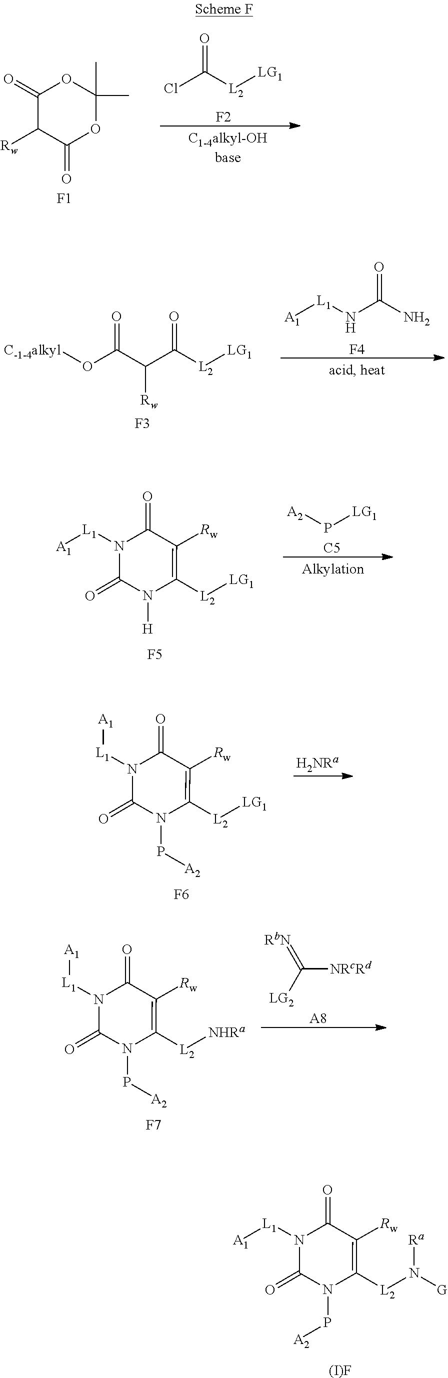

A compound of formula F5 may be alkylated with an alkylating agent C5 using conventional chemistry known to one versed in the art to prepare a compound of formula F6. A nucleophilic displacement of LG₁ with amine H₂NR^{a} affords a compound of formula F7, which may be further elaborated to include a G-substituent using the methods described herein to give a compound (I)F of Formula (I).

Scheme G illustrates the general synthesis of compounds of the present invention wherein W is N and L₂ is —X₂—(CH₂)₀₋₄—. A compound of formula G1 (either commercially available or prepared by known methods described in the scientific literature) may be treated with a base followed by alkylation with a compound of formula A4 to afford a compound of formula G2. Treatment of a compound of formula G2 with an aqueous base such as sodium hydroxide gives a compound of formula G3, which upon treatment with ammonia or its equivalent provides a compound of formula G4. The compound of formula G4 may then be condensed with a compound of formula G5 to form a triazine compound of formula G6.

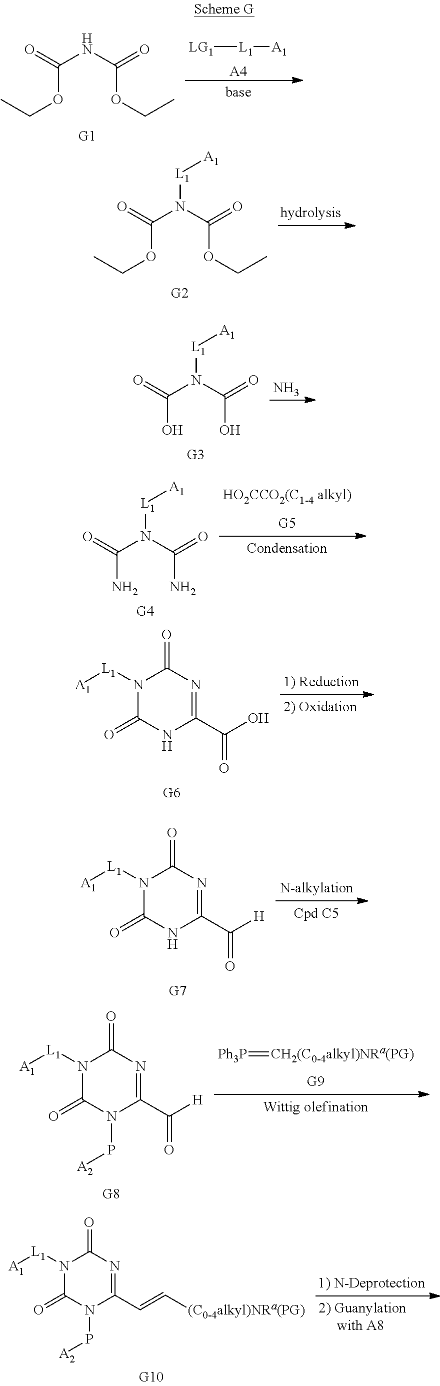

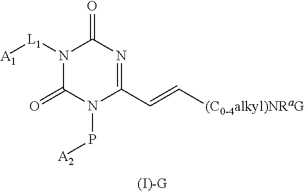

Using conventional reagents and methods known to one skilled in the art, the carboxy group of compounds of G6 may be reduced to the corresponding alcohol, followed by oxidation to an aldehyde of formula G7. The secondary amino group may be substituted with a compound of formula C5 using coupling chemistry or standard alkylation chemistry to afford a compound of formula G8. The aldehyde portion of the compound may participate in a Wittig olefination with a compound of formula G9 (wherein PG is as previously defined) to provide a compound of formula G10 wherein L₂ includes an alkenyl group, X₂. Subsequent removal of the amino-protecting group followed by guanylation gives a compound of Formula (I)G.

Scheme H illustrates the general synthesis of compounds of the present invention wherein W is CH and L₂ is —X₂—(CH₂)₀₋₄—. A compound of formula H1 may be condensed with an O-alkylated isourea to afford a cyclic compound of formula H2. The amine may be deprotonated with an organometallic base and subsequently treated with a compound of formula A4 to install the -L₁A₁ substituents of Formula (I). O-demethylation of the alkylated compounds of formula H2 afford compounds of formula H3. Using conventional oxidation chemistry, the methyl substituent of H3 may be converted to its corresponding aldehyde, affording a compound of formula H4. The aldehyde may be elaborated to a compound of Formula (I) wherein L₂ is —X₂—(CH₂)₀₋₄— using the synthetic steps described in Scheme G for the conversion of a compound G7 to compounds of Formula (I)G.

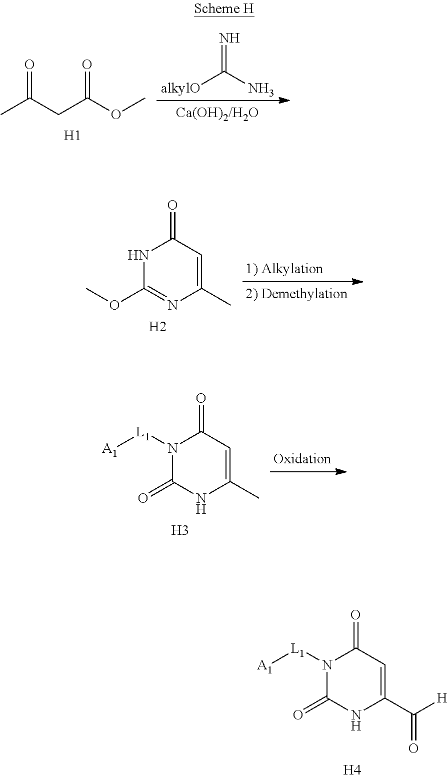

Scheme I depicts the general synthesis of compounds of the present invention wherein L₂ of Formula (I) is one which contains an X₁ group, and W is N. In Scheme I, X₁ is S.

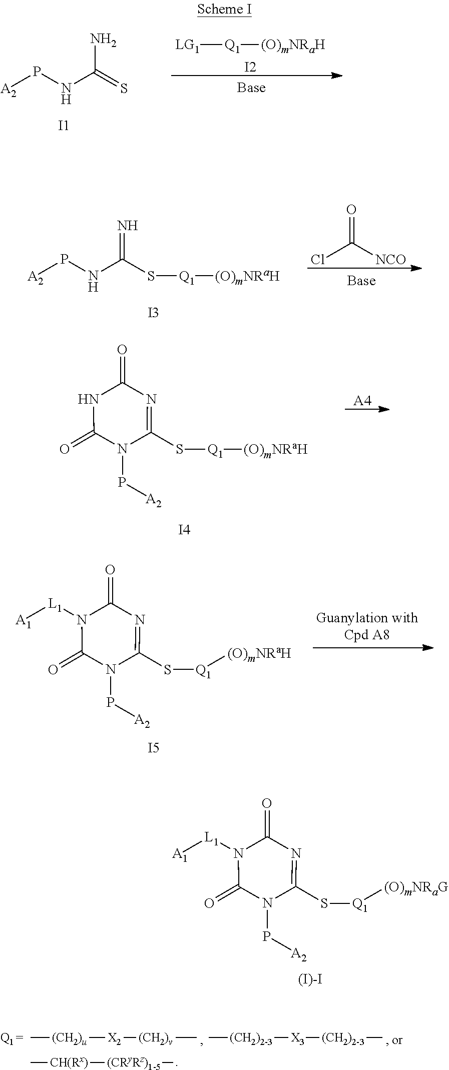

A compound of formula I1 (either commercially available or prepared by known methods described in the scientific literature) may be alkylated under basic conditions with a compound of formula I2 (wherein Q₁ is —(CH₂)ᵤ—X₂—(CH₂)ᵥ—, —(CH₂)₂₋₃—X₃—(CH₂)₂₋₃—, or —CH(R^{x})—(CR^{y}R^{z})₁₋₅—) to provide a compound of formula I3. A compound of formula I3 may be condensed with an appropriately substituted isocyanate such as N-chlorocarbonyl isocyanate in the presence of excess tertiary amine such as diisopropylethylamine to give a triazine of formula I4. A compound of formula I4 may be alkylated with a compound of formula A4 to provide a compound of formula I5, which may then be guanylated according the methods described herein to provide a compound of Formula (I)-I.

Scheme J illustrates the general synthesis of compounds of the present invention wherein L₂ is —C(═O)NH(CR^{y}R^{z})₂₋₅— and W is N.

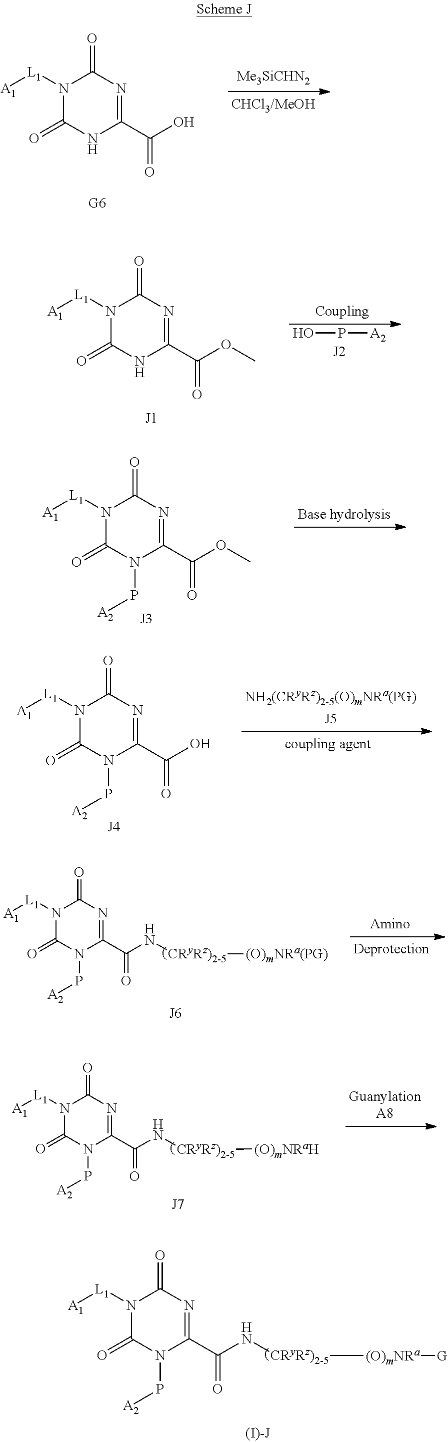

A compound of Formula G6 may be treated with a methylating agent such as trimethylsilyl diazomethane to give the methyl ester of formula J1. Under Mitsunobu type coupling conditions (in the presence of a coupling agent, activating agent), an alcohol of formula J2 may be coupled with the secondary amine of a compound of formula J1 to afford a compound of formula J3. Standard base hydrolysis of the methyl ester gives a compound of formula J4, wherein the corresponding carboxylic acid may be coupled with an amine of formula J5 (PG is an appropriate amino protecting group) to afford a compound of formula J6. Standard removal of the amino protecting group, PG, yields the primary amine of formula J7, which may be guanylated according to the methods described herein to yield a compound of Formula (I)-J.

Scheme K illustrates the general synthesis of compounds of the present invention wherein L₂ is —C(═O)NH(CR^{y}R^{z})₂₋₅— and W is CH.

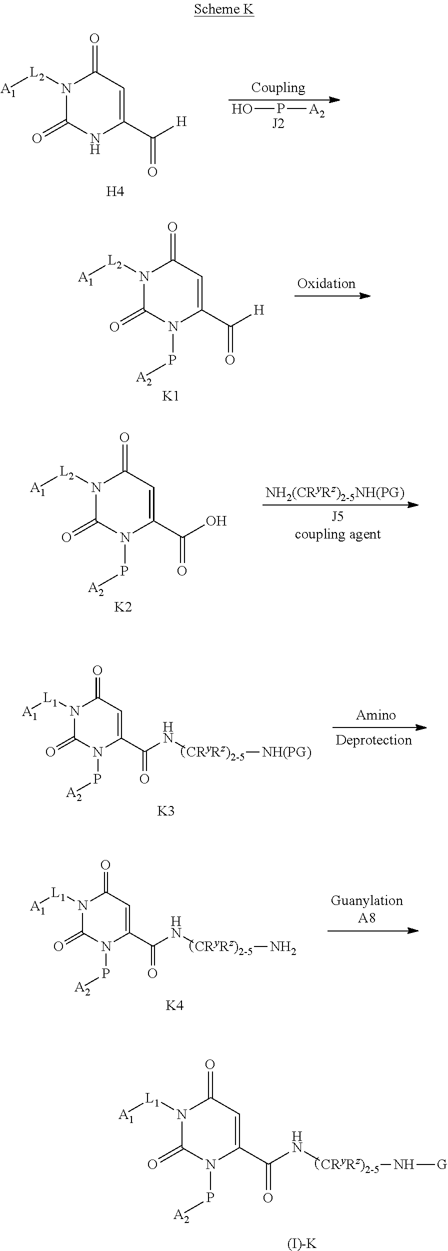

A compound of formula H4 may be treated under Mitsunobu-type coupling conditions (in the presence of a coupling agent and activating agent), with an alcohol of formula J2 to afford a compound of formula K1. Oxidation of the aldehyde group using an appropriate oxidizing agent gives a compound of formula K2, wherein the corresponding carboxylic acid may be coupled with an amine of formula J5 (PG is an appropriate amino protecting group) to afford a compound of formula K3. The conventional removal of the amino protecting group, PG, yields the primary amine of formula K4, which may be guanylated according to the methods described herein to yield a compound of Formula (I)-K.

### SPECIFIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The instant compounds may also be used as intermediates in subsequent examples to produce additional compounds of the present invention. No attempt has been made to optimize the yields obtained in any of the reactions. One skilled in the art would know how to increase such yields through routine variations in reaction times, temperatures, solvents and/or reagents.

Reagents were purchased from commercial sources. Nuclear magnetic resonance (NMR) spectra for hydrogen atoms were measured in the indicated solvent with (TMS) as the internal standard on a Bruker-Biospin Inc. DRX 500 (500 MHz) or DPX 300 (300 MHz) spectrometer. The values are expressed in parts per million downfield from TMS. The mass spectra (MS) were determined on a Micromass Platform LC spectrometer, an Agilent LC spectrometer or a Micromass LCT spectrometer using electrospray techniques. Microwave accelerated reactions were performed using a CEM Discover microwave instrument, and were contained in a sealed pressure vessel unless otherwise noted. Stereoisomeric compounds may be characterized as racemic mixtures or as separate diastereomers and enantiomers thereof using X-ray crystallography and other methods known to one skilled in the art. Unless otherwise noted, the materials used in the examples were obtained from readily available commercial suppliers or synthesized by standard methods known to one skilled in the art of chemical synthesis. The substituent groups, which vary between examples, are hydrogen unless otherwise noted.

### Example 1

### N-{2-[5-(4-Ethyl-benzyl)-1-(4-methoxy-benzyl)-4,6-dioxo-1,4,5,6-tetrahydro-[1,3,5]triazin-2-ylamino]-ethyl}-guanidine (Cpd 46)

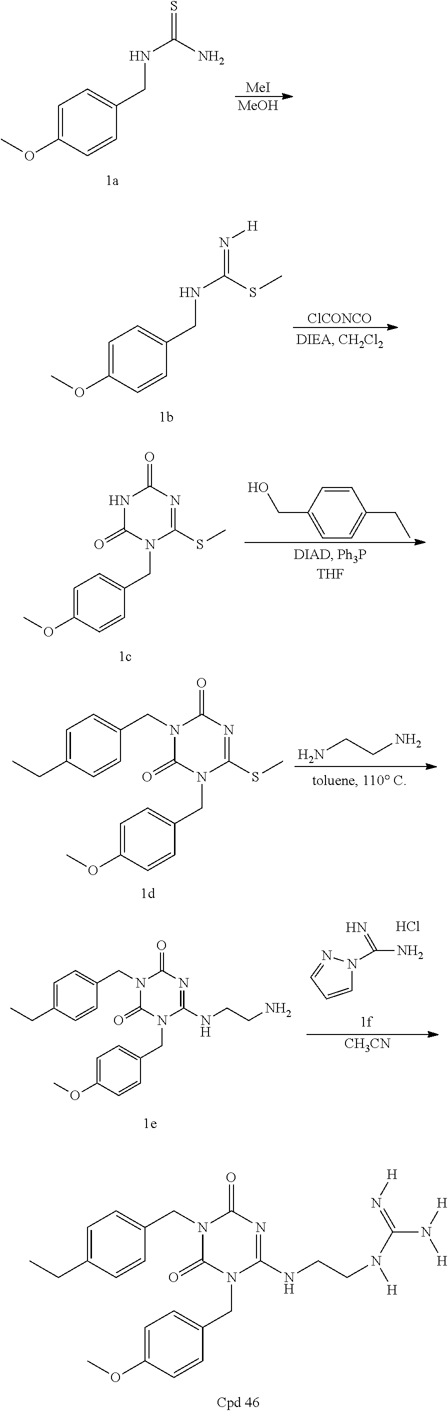

A. 1-(4-Methoxy-benzyl)-6-methylsulfanyl-1H-[1,3,5]triazine-2,4-dione (Cpd_{—}1c). To (4-methoxy-benzyl) thiourea (2.00 g, 10 1 mmol) in MeOH (40 mL) was added methyl iodide (0.64 mL, 10.1 mmol). The reaction was stirred at room temperature for 24 h. The reaction mixture was concentrated to yield 2.00 g of crude compound (1b) that was used in the next step without further purification.

B. To Compound 1b (3.6 g, 17 1 mmol) in methylene chloride (40 mL) was added excess diisopropylethylamine (6.61 g, 51.3 mmol). The reaction mixture was cooled to 0° C. A portion of N-chlorocarbonyl isocyanate (1.78 g, 17.1 mmol) was added dropwise. The reaction mixture was allowed to slowly warm to room temperature. After 24 h, water was added and the reaction mixture was extracted with ethyl acetate. The phases were separated, and the organic layer was dried over sodium sulfate, filtered, and concentrated. Methanol was added to the crude product, and the solid was collected by vacuum filtration to give Compound 1c (1.5 g). ¹H NMR (DMSO-d₆) δ 2.45 (3H, s), 3.73 (3H, s), 4.98 (2H, s), 6.89-6.92 (2H, d, J=8.5 Hz), 7.22-7.25 (2H, d, J=8.5 Hz), 11.58 (1H, s).

C. 3-(4-Ethyl-benzyl)-1-(4-methoxy-benzyl)-6-methylsulfanyl-1H-[1,3,5]triazine-2,4-dione (Cpd 1d). To Cpd 1c (0.1 g, 0.35 mmol) in tetrahydrofuran was added 4-ethylbenzyl alcohol (0.049 g, 0.35 mmol), triphenylphosphine (0.19 g 0.71 mmol) and diisopropyl azodicarboxylate (0.087 g, 0.43 mmol). The reaction stirred at room temperature for 64 h. The reaction mixture was taken up in ethyl acetate, washed with water, and the phases were separated. The organic layer was dried over sodium sulfate, filtered, and concentrated. The resulting material was purified by normal phase chromatography using an ISCO automated system to give Cpd 1d (0.14 g) as a white solid.

D. 6-(2-Amino-ethylamino)-3-(4-ethyl-benzyl)-1-(4-methoxy-benzyl)-1H-[1,3,5]triazine-2,4-dione (Cpd 1e). To 1-(4-methoxy-benzyl)-6-methylsulfanyl-1H-[1,3,5]triazine-2,4-dione (0.14 g, 0.33 mmol) in toluene was added excess ethylenediamine (0.10 g, 1.76 mmol). The reaction mixture was heated at 110° C. for 18 h. The reaction mixture was cooled to room temperature, diluted with water and extracted with ethyl acetate. The phases were separated and the organic layer was dried over sodium sulfate, filtered and concentrated. The resultant Cpd 1e (0.11 g) was used in the next step without further purification.

E. N-{2-[5-(4-Ethyl-benzyl)-1-(4-methoxy-benzyl)-4,6-dioxo-1,4,5,6-tetrahydro-[1,3,5]triazin-2-ylamino]-ethyl}-guanidine (Cpd 46). To a mixture of Cpd 1e (0.11 g, 0.26 mmol) in acetonitrile (4 mL) was added excess diisopropylamine (0.069 g, 0.53 mmol) and 1H-pyrazolo-1-carboxamidine hydrochloride, Cpd 1f, (0.039 g, 0.26 mmol). The reaction mixture was stirred for 18 h at room temperature. A white solid precipitated from the reaction mixture and was collected by filtration to give the title compound 46 (98% pure by HPLC, 0.0119 g). ¹H NMR (DMSO-d₆) δ 1.01-1.04 (3H, t, J=7.5 Hz), 2.41-2.47 (2H, q, J=7.4 Hz), 3.26-3.16 (4H, m), 3.61 (3H, s), 4.75 (2H, s), 4.93 (2H, s), 6.77-6.79 (2H, d, J=8.64 Hz), 7.00-7.12 (6H, m), 7.55 (1H, m), 8.06 (1H, m).

Using the procedures of Example 1 and the appropriate reagents, starting materials and purification methods known to those skilled in the art, the following compounds of the present invention were prepared: compounds 39, 45, 77, 78, 79, 80, 82, 83, 109, 111, 112, 123, 124, 131, 136, 137, 145, and 146.

### Example 2

### N-{2-[5-(4-Fluoro-benzyl)-1-(4-methoxy-benzyl)-4,6-dioxo-1,4,5,6-tetrahydro-[1,3,5]triazin-2-ylamino]-ethyl}-guanidine (Cpd 17)

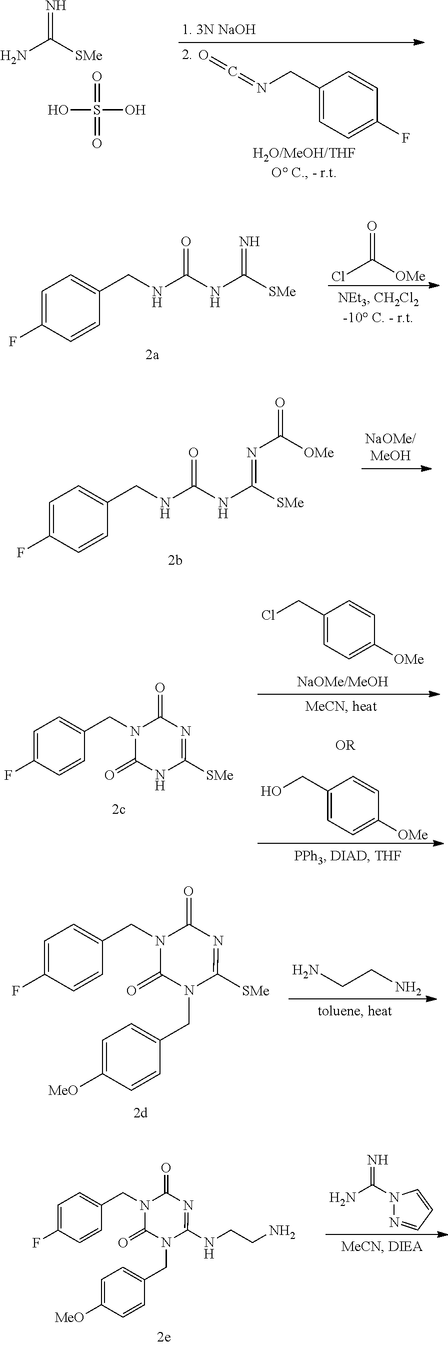

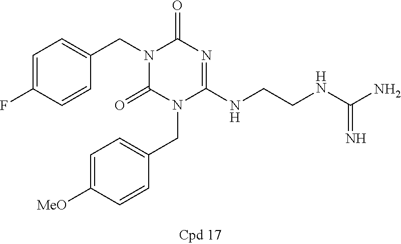

A. ((4-Fluorobenzyl)amino)carbonyl)carbamimidothioic acid methyl ester (Cpd 2a). S-methylisothiouronium sulfate (10.0 g, 35.9 mmol) was dissolved in 8:2:1 MeOH/H₂O/THF and the mixture was treated with 3 N NaOH (12 mL, 35.9 mmol). The solution was then cooled to 0° C. and 4-fluorobenzyl isocyanate (5.43 g, 35.9 mmol) was added dropwise over 30 min. The reaction was stirred overnight and gradually warmed to room temperature. The mixture was then washed with saturated aqueous NH₄Cl and extracted with dichloromethane. The combined organic phases were dried over Na₂SO₄, filtered and concentrated under reduced pressure. The resultant residue was purified on an Isco flash column (20% EtOAc-100% EtOAc in heptanes), to give Compound 2a (4.1 g) as a white powder.

B. 5-(Methylthio)-3,7-dioxo-1-(4-fluorobenzyl)-2-oxa-4,6,8-triazanon-4-en-9-oic acid methyl ester (Cpd 2b). A solution of Compound 2a (4.1 g, 17.0 mmol) in dichloromethane was treated with triethylamine (3.08 mL, 22.1 mmol) and the mixture was cooled to −10° C. Methyl chloroformate (2.62 mL, 34.0 mmol) was added dropwise via an addition funnel over 15 min and the reaction was allowed to stir for 4 h while gradually warming to room temperature. The solution was then washed with saturated aqueous NH₄Cl and extracted with dichloromethane. The combined organic phases were dried over Na₂SO₄, filtered and concentrated. The resultant residue was purified on an Isco flash column (5% MeOH) to afford Compound 2b (3.63 g) as a white solid.

C. 3-(4-Fluoro-benzyl)-6-methylsulfanyl-1H-[1,3,5]triazine-2,4-dione (Cpd 2c). Compound 2b (3.63 g, 12.1 mmol) was dissolved in MeOH (100 mL) and the solution was treated with NaOMe in MeOH (4.6 M, 2.90 mL, 13.3 mmol) and the reaction was allowed to stir at room temperature for 1 h. A white precipitate formed upon addition of the NaOMe. The reaction mixture was diluted with 1N HCl (50 mL) and the resultant precipitate was collected by filtration. The solid was dried under reduced pressure at 160° C. over xylenes to afford Compound 2c (3.6 g) as its HCl salt.

D. 3-(4-Fluoro-benzyl)-1-(4-methoxy-benzyl)-6-methylsulfanyl-1H-[1,3,5]triazine-2,4-dione (Cpd 2d). Compound 2c (500 mg, 1.65 mmol) was dissolved in THF and was treated with 4-methoxybenzyl alcohol (227 mg, 1.65 mmol), triphenylphospine (866 mg, 3.30 mmol), and diisopropyl azodicarboxylate (334 mg, 1.65 mmol). The reaction was allowed to stir overnight at room temperature. After monitoring the reaction via HPLC, the solution was partitioned between water and ethyl acetate. Combined organic layers were dried over anhydrous sodium sulfate, filtered and reduced. The crude mixture was purified via Isco flash column (20% ethyl acetate-100% ethyl acetate in heptanes, 40 min) to afford 390 mg of Cpd 2d as a white solid. ¹H NMR (DMSO, d₆). δ 3.29 (s, 3H), 3.74 (s, 3H), 4.93 (s, 2H), 5.03 (s, 2H), 6.89-6.92 (d, 2H, J=8.62), 7.12-7.36 (m, 4H), 7.38-7.41 (m, 2H).

E. 4-[3-(3,4-Dichloro-benzyl)-6-methylsulfanyl-2,4-dioxo-3,4-dihydro-2H-[1,3,5]triazin-1-ylmethyl]-benzamide (Cpd 2d). Compound 2c (dichorobenzyl) (200 mg, 0.56 mmol) was dissolved in MeCN and was treated with diisopropylethylamine (0.196 mL, 1.13 mmol) and 4-chloromethyl benzyl chloride (96 mg, 0.56 mmol). The reaction mixture was heated to 80° C. and was allowed to stir overnight. The reaction mixture was washed with saturated aqueous NH₄Cl and extracted with ethyl acetate. The combined organic extracts were dried over Na₂SO₄, filtered and concentrated. The resultant crude mixture was purified by Isco flash column (20%-100% EtOAc in heptanes, 40 min) to afford 70 mg of Cpd 2d as a white powder.

F. 6-(2-Amino-ethylamino)-3-(4-fluoro-benzyl)-1-(4-methoxy-benzyl)-1H-[1,3,5]triazine-2,4-dione (Cpd 2e). A solution of Compound 2d (390 mg, 1.01 mmol) in toluene (8 mL) and was treated with ethylenediamine (302 mg, 5.03 mmol). The reaction was heated to 90° C. and was allowed to stir overnight. The mixture was then partitioned between water and ethyl acetate. The combined organic layers were dried over Na₂SO₄, filtered and reduced. Reduction provided 390 mg of Cpd 2e as a crude mixture. The crude compound was used in further synthesis without additional purification.

G. N-{2-[5-(4-Fluoro-benzyl)-1-(4-methoxy-benzyl)-4,6-dioxo-1,4,5,6-tetrahydro-[1,3,5]triazin-2-ylamino]-ethyl}-guanidine (Cpd 17). A crude mixture of Cpd 2e (390 mg, 0.98 mmol) was dissolved in acetonitrile (10 mL) and was treated with pyrazole-1-carboxamidine hydrochloride (143 mg, 0.98 mmol) and diisopropylethylamine (0.340 mL, 1.95 mmol). The reaction was allowed to proceed overnight at room temperature. Inspection of the reaction mixture showed that a white precipitate had formed and the precipitate was collected and dried by vacuum filtration. The solid collected afforded 307 mg of Cpd 17 as a white powder. M⁺ (ES+)=442.3. ¹H NMR (DMSO, d₆). δ 3.33 (m, 4H), 3.73 (s, 3H), 4.89 (s, 2H), 5.04 (s, 2H), 6.89-6.91 (d, 2H, J=8.66 Hz), 7.10-7.16 (t, 2H, J=8.91 Hz), 7.21-7.24 (d, 2H, J=8.63 Hz), 7.32-7.36 (dd, 2H, J=2.90, 5.57 Hz), 7.66 (s, 1H), 8.19 (s, 1H).

Using the procedures of Example 2 and the appropriate reagents, starting materials and purification methods known to those skilled in the art, the following compounds of the present invention were prepared: compounds 1, 2, 3, 4, 5, 6, 7, 8, 9, 11, 12, 13, 14, 15, 16, 18, 19, 20, 21, 22, 23, 24, 25, 25, 29, 30, 31, 32, 33, 34, 35, 36, 37, 38, 40, 41, 50, 51, 52, 57, 68, 69, 85, 86, 87, 129, 130, 142, 144, 147, 148, 149, and 150.

Cpd 51: 4-[3-(3,4-Dichlorobenzyl)-6-(2-guanidinoethylamino)-2,4-dioxo-3,4-dihydro-2H-[1,3,5]triazin-1-ylmethyl]-benzamide δ (DMSO, d₆) 3.30-3.37 (m, 4H), 4.90 (s, 2H), 5.10 (s, 1H), 7.27-7.32 (m, 3H), 7.51-7.61 (m, 2H), 7.83 (d, 2H, J=9.7 Hz), 7.94 (s, 1H), 8.08 (t, 1H, J=3.7 Hz).

### Example 3

### N-{2-[1-Benzyl-3-(4-methoxy-benzyl)-2,6-dioxo-1,2,3,6-tetrahydro-pyrimidin-4-ylamino]-ethyl}-guanidine (Cpd 81)

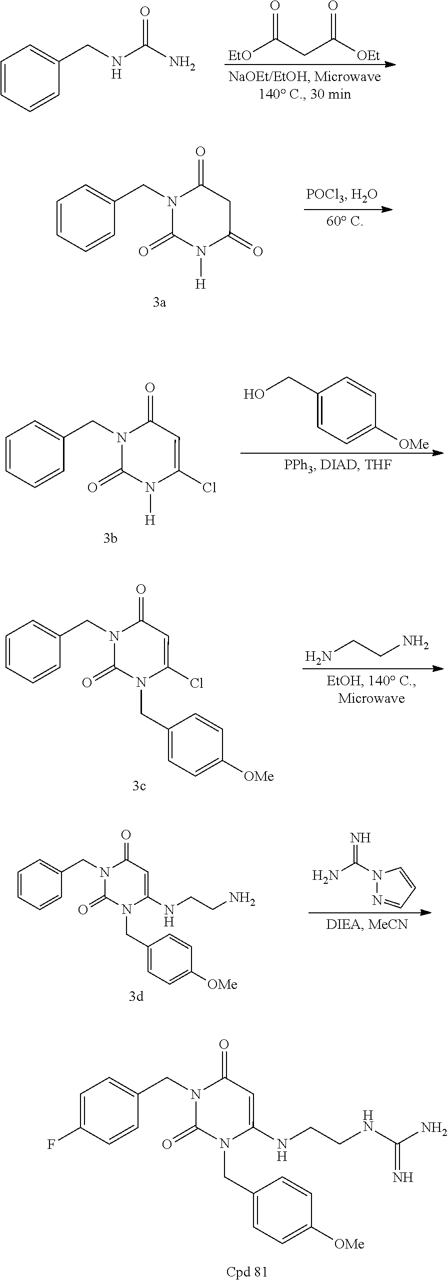

A. 1-Benzyl-pyrimidine-2,4,6-trione (Cpd 3a). N-benzyl urea (500 mg, 3.33 mmol) was dissolved in ethanol (8 mL) and the mixture was treated with diethyl malonate (640 mg, 4.0 mmol) and NaOEt in EtOH (1.29 mL, 3.1M, 4.0 mmol). The reaction was then run under microwave conditions at 140° C. for 30 min. The solution was reduced in vacuo and the residue was triturated with ethanol. The desired compound was collected by vacuum filtration to give Cpd 3a (500 mg) as a white powder. ¹H NMR (DMSO, d₆). δ 3.69 (s, 2H), 4.87 (s, 2H), 7.21-7.31 (m, 5H) 11.41 (s, 1H).

B. 6-Chloro-3-benzyl uracil (Cpd 3b). Cpd 3a (500 mg, 2.29 mmol) was dissolved in phosphorous oxychloride (3.5 mL, 22.9 mmol) and the reaction mixture was cautiously treated with water (0.103 mL, 5.7 mmol). The solution was heated to 60° C. and was stirred overnight. The reaction mixture was then concentrated and the residue was poured over 2N NaOH (15 mL). The crude material was collected by vacuum filtration and purified by recrystallization from ethanol to afford Cpd 3b (60 mg) as a white powder. A second crop of 300 mg of crude 3b was recovered from the recrystallization and used in subsequent reactions without further purification. ¹H NMR (MeOD, d₄). δ 5.04 (s, 2H), 5.87 (s, 1H), 7.25-7.38 (m, 5H).

C. 1-(4-Methoxylbenzyl)-6-chloro-3-benzyl uracil (Cpd 3c). A stirred solution of Cpd 3b (60 mg, 0.25 mmol) in THF was treated with 4-methoxylbenzyl alcohol (35 mg, 0.25 mmol), triphenylphosphine (133 mg, 0.51 mmol) and diisopropyl azocarboxylate (51 mg, 0.25 mmol). The reaction was allowed to stir overnight at room temperature. The mixture was washed with water and extracted with ethyl acetate. Combined organic extracts were dried over Na₂SO₄, filtered and concentrated. The resultant residue was purified by Isco flash column chromatography (20% EtOAc-100 EtOAc in heptanes, 40 min) to afford Cpd 3c (60 mg) as a white powder. M⁺(ES+)=356.9.

D. 6-(2-Amino-ethylamino)-3-benzyl-1-(4-methoxybenzyl)-uracil (Cpd 3d). Cpd 3c (60 mg, 0.17 mmol) was dissolved in ethanol (3 mL) and the reaction mixture was treated with ethylenediamine (51 mg, 0.84 mmol). The solution was run at 140° C. for 20 min under power max conditions in a microwave reactor. The solution was washed with water and extracted with ethyl acetate. Combined organic phases were dried over Na₂SO₄, filtered and concentrated to give crude Cpd 3d (35 mg) as a yellow oil. The crude mixture was used in subsequent reactions without further purification.

E. N-{2-[1-Benzyl-3-(4-methoxy-benzyl)-2,6-dioxo-1,2,3,6-tetrahydro-pyrimidin-4-ylamino]-ethyl}-guanidine (Cpd 81). The title compound was prepared as described in Example 2, Step G. The crude material was purified by reverse phase preparative HPLC to give the title compound as its TFA salt (8.2 mg). M+ (ES+)=422.9. ¹H NMR (MeOD, d₄). δ 3.19-3.24 (m, 4H), 3.67 (s, 3H), 4.77 (s, 1H), 4.99 (s, 2H), 5.03 (s, 2H), 6.77-6.80 (d, 2H, J=8.79 Hz), 7.01-7.04 (d, 2H, J=8.75 Hz), 7.12-7.25 (m, 5H).

Using the procedures of Example 3 and the appropriate reagents, starting materials and purification methods known to those skilled in the art, the following compounds of the present invention were prepared: compound 84.

Cpd 84: N-{2-[1,3-Bis-(4-methoxy-benzyl)-2,6-dioxo-1,2,3,6-tetrahydro-pyrimidin-4-ylamino]-ethyl}-guanidine (DMSO, d₆) δ 3.25-3.27 (m, 2H), 3.35-3.37 (m, 2H), 3.74 (s, 3H), 3.75 (s, 3H), 4.83 (s, 1H), 4.90 (s, 2H), 5.15 (s, 2H), 6.81-6.89 (m, 4H), 7.14-7.24 (m, 4H), 7.70 (s, 1H).

### Example 4

### N-{2-[5-(4-Fluoro-benzyl)-1-(4-methoxy-benzyl)-4,6-dioxo-1,4,5,6-tetrahydro-[1,3,5]triazin-2-ylamino]-ethyl}-N′-(4-fluoro-phenyl)-guanidine (Cpd 119)

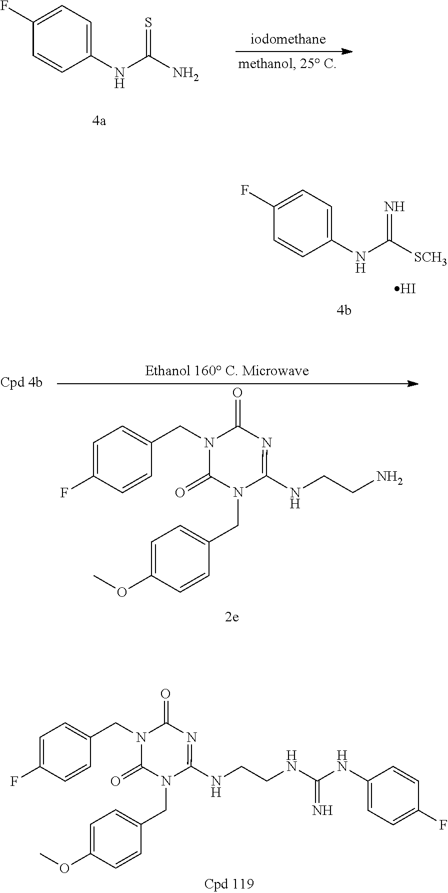

A. 1-(4-Fluoro-phenyl)-2-methyl-isothiourea (Cpd. 4b). To a solution of (4-Fluoro-phenyl)-thiourea (18.7 mg, 0.11 mmol) and methanol (0.25 mL) was added iodomethane (8 μL, 0.13 mmol). The mixture was stirred at 25° C. for 16 h, then concentrated to a residue to provide crude compound 4b.

C. N-{2-[5-(4-Fluoro-benzyl)-1-(4-methoxy-benzyl)-4,6-dioxo-1,4,5,6-tetrahydro-[1,3,5]triazin-2-ylamino]-ethyl}-N′-(4-fluoro-phenyl)-guanidine (Cpd 127). To a solution of Compound 4b in ethanol (0.5 mL) was added Compound 2e (40 mg, 0.10 mmol). The mixture was irradiated in a microwave reactor at 160° C. for 15 min, then concentrated. The resulting residue was dissolved into dimethylsulfoxide and purified by reversed-phase chromatography to furnish the title compound 119 (18.3 mg, 0.024 mmol) as its TFA salt. ¹H NMR (methanol-d₄): δ 7.42 (m, 2H), 7.24-7.12 (m, 6H), 7.00 (m, 2H), 6.89 (m, 2H), 5.06 (s, 2H), 5.01 (s, 2H), 3.75 (s, 3H), 3.56 (m, 2H), 3.43 (m, 2H); HRMS m/z (M+H)⁺ calcd 536.2222, found 536.2227.

Using the procedures of Example 4 and the appropriate reagents, starting materials and purification methods known to those skilled in the art, the following compounds of the present invention were prepared: compounds 44, 53, 54, 58, 61, 62, 63, 64, 65, 66, 67, 70, 71, 72, 73, 74, 75, 76, 88, 89, 90, 91, 92, 103, 104, 105, 106, 107, 108, 114, 115, 116, 117, 118, 120, 121, 126, 127, 128, 133, 134, 135, 138, 139, 140, 151, 152, 153, 154, 155, and 156.

Cpd 58: N-{2-[5-(3,4-Dichloro-benzyl)-1-(4-methoxy-benzyl)-4,6-dioxo-1,4,5,6-tetrahydro-[1,3,5]triazin-2-ylamino]-ethyl}-N-isopropyl-guanidine. ¹H NMR (methanol-d₄): 7.56 (s, 1H), 7.45 (d, 1H, J=8.3 Hz), 7.35 (d, 1H, J=8.3 Hz), 7.22 (d, 2H, J=8.3 Hz), 6.89 (d, 2H, J=8.4 Hz), 5.12 (s, 2H), 5.01 (s, 2H), 3.77 (s, 3H), 3.68 (m, 1H), 3.57 (t, 2H, J=6.3 Hz), 3.41 (t, 2H, J=6.3 Hz), 1.17 (d, 6H, J=6.5 Hz); HRMS m/z (M+H)⁺ calcd 534.1787, found 534.1792.

Cpd 90: N-(4-Cyano-phenyl)-N-{2-[5-(4-fluoro-benzyl)-1-(4-methoxy-benzyl)-4,6-dioxo-1,4,5,6-tetrahydro-[1,3,5]triazin-2-ylamino]-ethyl}-guanidine. ¹H NMR (methanol-d₄): 7.74 (d, 2H, J=8.7 Hz), 7.44 (m, 2H), 7.35 (d, 2H, J=8.3 Hz), 7.21 (d, 2H, J=8.6 Hz), 7.01 (t, 2H, J=8.8 Hz), 6.88 (d, 2H, J=8.8 Hz), 5.11 (s, 2H), 5.02 (s, 2H), 3.75 (s, 3H), 3.61 (t, 2H, J=6.3 Hz), 3.51 (m, 2H); HRMS m/z (M+H)⁺ calcd 543.2268, found 543.2273.

Cpd 104: N-{2-[5-(4-Fluoro-benzyl)-1-(4-methoxy-benzyl)-4,6-dioxo-1,4,5,6-tetrahydro-[1,3,5]triazin-2-ylamino]-ethyl}-N′-pyridin-2-yl-guanidine. ¹H NMR (DMSO-d₆): δ10.90 (br, 1H), 9.78 (br, 1H), 8.65 (br, 2H), 8.17 (d, 1H, J=5.4 Hz), 8.07 (m, 1H), 7.87 (t, 1H, J=7.8 Hz), 7.33 (m, 2H), 7.13 (m, 4H), 7.05 (d, 1H, J=8.2 Hz), 6.78 (d, 2H, J=8.7 Hz), 4.98 (s, 2H), 4.86 (s, 2H), 3.67 (s, 3H), 3.54 (m, 2H), 3.36 (br, 2H); HRMS m/z (M+H)⁺ calcd 519.2268, found 519.2253.

Cpd 118: N-{2-[5-(4-Fluoro-benzyl)-1-(4-methoxy-benzyl)-4,6-dioxo-1,4,5,6-tetrahydro-[1,3,5]triazin-2-ylamino]-ethyl}-N′-(2-fluoro-phenyl)-guanidine. ¹H NMR (methanol-d₄): δ 7.47-7.37 (m, 3H), 7.31 (t, 1H, J=7.8 Hz), 7.23 (m, 2H), 7.18 (d, 2H, J=8.6 Hz), 7.01 (t, 2H, J=8.8 Hz), 6.89 (d, 2H, J=8.8 Hz), 5.06 (s, 2H), 5.01 (s, 2H), 3.76 (s, 3H), 3.56 (t, 2H, J=6.3 Hz), 3.45 (t, 2H, J=6.3 Hz); HRMS m/z (M+H)⁺ calcd 536.2222, found 536.2227.

Cpd 134: N-Benzoyl-N′-{2-[5-(4-fluoro-benzyl)-1-(4-methoxy-benzyl)-4,6-dioxo-1,4,5,6-tetrahydro-[1,3,5]triazin-2-ylamino]-ethyl}-guanidine. ¹H NMR (methanol-d₄): δ 7.93 (d, 2H, J=8.2 Hz), 7.70 (t, 1H, J=7.5 Hz), 7.57 (t, 2H, J=7.5 Hz), 7.41 (m, 2H), 7.16 (d, 2H, J=8.7 Hz), 6.97 (t, 2H, J=8.7 Hz), 6.85 (d, 2H, J=8.7 Hz), 5.08 (s, 2H), 4.99 (s, 2H), 3.70 (s, 3H), 3.66 (t, 2H, J=6.2 Hz), 3.55 (t, 2H, J=6.2 Hz); HRMS m/z (M+H)⁺ calcd 546.2265, found 546.2259.

### Example 5

### N-{2-[5-Benzyl-1-(4-methoxy-benzyl)-4,6-dioxo-1,4,5,6-tetrahydro-[1,3,5]triazin-2-ylamino]-ethyl}-oxyguanidine (Cpd 27)

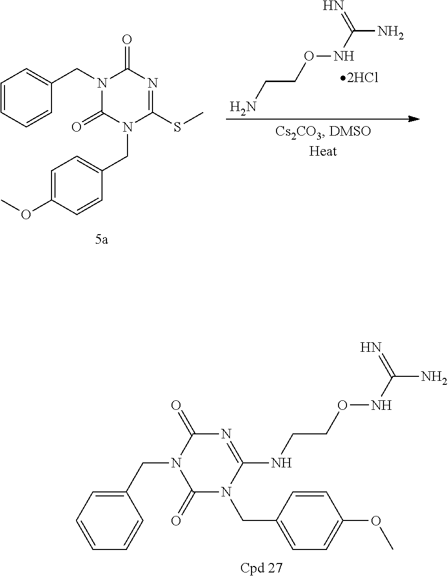

A. Compound 5a was prepared by the method described in Example 1, Step C, substituting phenyl methanol for 4-ethylbenzyl alcohol.

B. To 3-benzyl-1-(4-methoxy-benzyl)-6-methylsulfanyl-1H-[1,3,5]triazine-2,4-dione 5a (0.056 g, 0.15 mmol) in DMSO (1 mL) was added N-(2-amino-ethyl)-oxyguanidine dihydrochloride salt (0.058 g, 0.30 mmol) and Cs₂CO₃ (0.098 mg, 0.30 mmol). The reaction mixture was heated at 70° C. for 5 h and cooled to rt. N-(2-Amino-ethyl)-oxyguanidine dihydrochloride salt (0.058 g, 0.30 mmol) and Cs₂CO₃ (0.098 mg, 0.30 mmol) were again added and the resulting slurry stirred at 40° C. for 16 h. The reaction mixture was cooled to room temperature, loaded onto a 1 g C-18 SPE cartridge, and eluted with CH₃CN. The eluant was concentrated and the resulting residue was purified by reverse-phase liquid chromatography using a gradient of 90:10 (acetonitrile:water, with 0.1% TFA) to 90:10 (acetonitrile: water, with 0.1% TFA) to give the title compound 27 (99% pure by HPLC, 0.0289 g). ¹H NMR (d⁶-DMSO/CDCl₃) δ 3.65-3.73 (2H, m), 3.78 (3H, s), 3.96-4.04 (2H, m), 5.01 (2H, s), 5.10 (2H, s), 6.85 (2H, d, J=8.7 Hz), 7.21-7.40 (7H. m), 7.74 (4H, bs); 7.89 (1H, m) 11.58 (1H, bs); HRMS calcd. for C₂₁H₂₆N₇O₄ m/z 440.2046 (M+H), found: 440.2030.

Using the procedures of Example 5 and the appropriate reagents, starting materials and purification methods known to those skilled in the art, the following compounds of the present invention were prepared: compound 10.

### Example 6

### 4-[4-(2-Guanidino-ethylamino)-3-(4-methoxy-benzyl)-2,6-dioxo-3,6-dihydro-2H-[1,3,5]triazin-1-ylmethyl]-benzoic acid (Cpd 101)

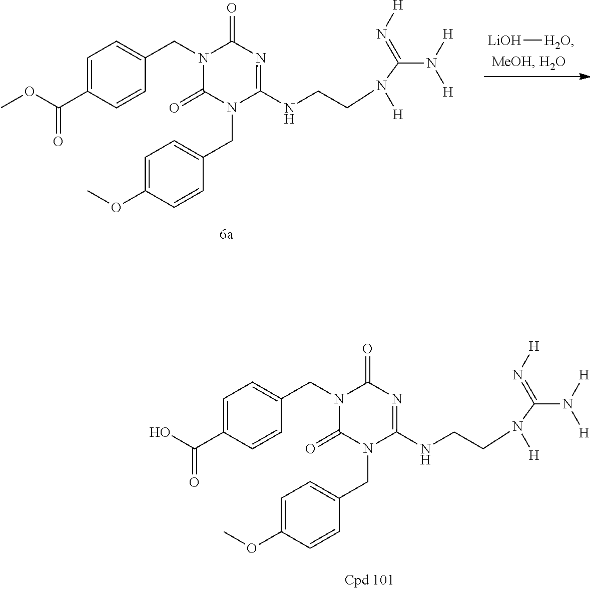

A. Compound 6a was prepared according to the methods described in Example 1, and substituting 4-hydroxymethyl-benzoic acid methyl ester for 4-ethylbenzyl alcohol.

B. 4-[4-(2-Guanidino-ethylamino)-3-(4-methoxy-benzyl)-2,6-dioxo-3,6-dihydro-2H-[1,3,5]triazin-1-ylmethylpbenzoic acid (Cpd. 101). A mixture of compound 6a (20 mg, 0.028 mmol) and lithium hydroxide (6 mg, 0.014 mmol) in 5 mL of MeOH and 1 mL of H₂O was allowed to stir overnight at room temperature. At that time, an additional 6 mg of lithium hydroxide was added and the mixture stirred for and additional 18 h. The mixture was then concentrated and purified by HPLC. The title compound 101 was obtained as its TFA salt (10 mg, 0.014 mmol). ¹H NMR (DMSO-d₆) δ 3.26 (m, 2H), 3.40 (m, 2H), 3.68 (s, 3H), 4.97 (s, 2H), 5.02 (s, 2H), 6.79-6.82 (d, 2H, J=8.7 Hz), 7.06-7.09 (d, 2H, J=8.7 Hz), 7.35-7.38 (d, 2H, J=8.2 Hz), 7.86-7.88 (d, 2H, J=8.3 Hz).

### Example 7

### N-{2-[5-(4-Hydroxy-benzyl)-1-(4-methoxy-benzyl)-4,6-dioxo-1,4,5,6-tetrahydro-[1,3,5]triazin-2-ylamino]-ethyl}-guanidine (Cpd 110)

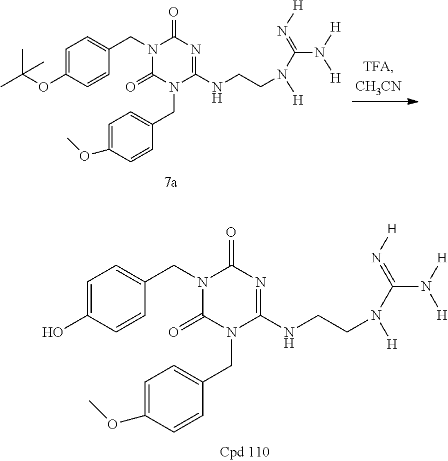

A. Compound 7a was prepared according to the methods described in Example 1, and substituting (4-tert-butoxy-phenyl)-methanol) for 4-ethylbenzyl alcohol.

B. N-{2-[5-(4-Hydroxy-benzyl)-1-(4-methoxy-benzyl)-4,6-dioxo-1,4,5,6-tetrahydro-[1,3,5]triazin-2-ylamino]-ethyl}-guanidine (Cpd 110). The crude Compound 7a (assumed to be about 0.24 mmol) was dissolved in CH₃CN. To this mixture was added 3 mL of TFA. The resulting mixture was allowed to stir overnight at room temperature. The mixture was concentrated and purified by HPLC to give the title compound 110 as its TFA salt (31 mg, 0.046 mmol). ¹H NMR (DMSO-d₆) δ 1.25-1.28 (m, 1H), 3.28-2.31 (m, 2H), 3.31-3.36 (m, 2H), 3.73 (s, 3H), 4.78 (s, 2H), 4.98 (s, 2H), 6.65-6.68 (d, 2H, J=8.4 Hz), 6.89-6.91 (d, 2H, J=8.7 Hz), 7.11-7.14 (d, 2H, J=8.6 Hz), 7.52-7.54 (d, 2H, J=5.5 Hz), 7.99 (m, 1H).

### Example 8

### N-{2-[1-(4-Methoxy-benzyl)-5-(4-nitro-benzyl)-4,6-dioxo-1,4,5,6-tetrahydro-[1,3,5]triazin-2-ylamino]-ethyl}-guanidine (Cpd 95)

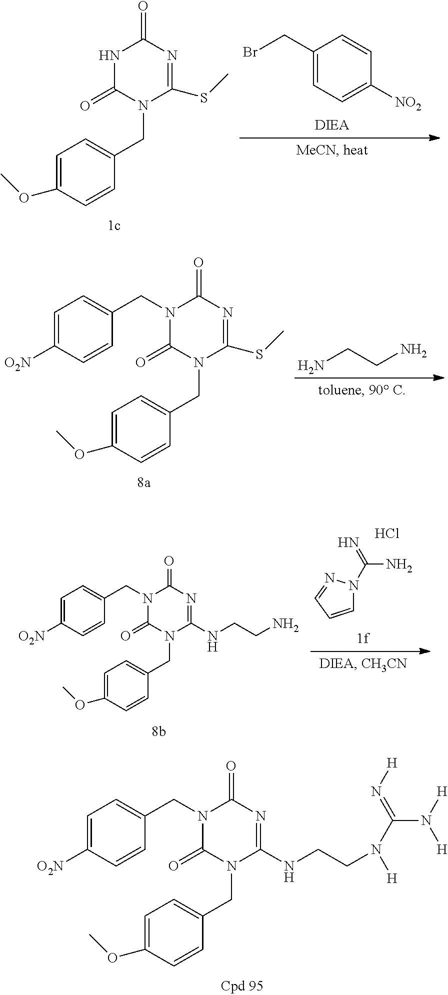

A. 1-(4-Methoxy-benzyl)-6-methylsulfanyl-3-(4-nitro-benzyl)-1H-[1,3,5]triazine-2,4-dione (Cpd 9a). Compound 1c (200 mg, 0.73 mmol) was dissolved in CH₃CN and was treated with 4-nitrobenzyl bromide (168 mg, 0.86 mmol) and 80 μL (0.73 mmol) of diisopropylethylamine The resulting mixture was heated to 87° C. and allowed to stir overnight. The reaction mixture was cooled to room temperature, diluted with ethyl acetate, and washed with saturated sodium bicarbonate solution. The organic phase was dried over MgSO₄, filtered, and concentrated. The residue was purified by flash chromatography to give compound 8a (44 g, 0.36 mmol).

B. 6-(2-Amino-ethylamino)-1-(4-methoxy-benzyl)-3-(4-nitro-benzyl)-1H-[1,3,5]triazine-2,4-dione (Cpd. 9b). To compound 8a (80 mg, 0.19 mmol) in 10 mL of toluene was added an excess of ethylene diamine (64 μL, 0.95 mmol). The resulting mixture was heated to 90° C. for 26 h. The mixture was taken up in ethyl acetate and washed with water. The organic layer was separated, dried over MgSO₄ and concentrated. The crude product 8b (79mg, 0.18 mmol, 97% yield) was used in the next step without further purification.

C. N-{2-[1-(4-Methoxy-benzyl)-5-(4-nitro-benzyl)-4,6-dioxo-1,4,5,6-tetrahydro-[1,3,5]triazin-2-ylamino]-ethyl}-guanidine (Cpd 95). A mixture of compound 8b (51 mg, 0.12 mmol), 1H-pyrazole-1-carboxamidine hydrochloride (18 mg, 0.12 mmol), and diisopropylethylamine (26 μL, 0.36 mmol) in 10 mL of acetonitrile was allowed to stir at room temperature for several days. The resulting mixture was concentrated and purified by liquid chromatography. The title compound 95 was obtained as a white powder (17 mg, 0.036 mmol) and was submitted as a TFA salt. ¹H NMR (DMSO-d₆) δ 3.65-3.71 (m, 4H), 3.85 (s, 3H), 5.30 (bm, 4H), 6.99-7.02 (m, 2H), 7.26-7.30 (m, 2H), 7.54-7.60 (m, 2H), 8.02-8.20 (bs, 1H), 8.25 (m, 2H).

Using the procedures of Example 8 and the appropriate reagents, starting materials and purification methods known to those skilled in the art, the following compounds of the present invention were prepared: compounds 42, 43, 47, 55, 56, 59, 94, 97, 98, 99, 100, 102, and 113.

### Example 9

### N-{2-[5-(4-Amino-benzyl)-1-(4-methoxy-benzyl)-4,6-dioxo-1,4,5,6-tetrahydro-[1,3,5]triazin-2-ylamino]-ethyl}-guanidine (Cpd 125)

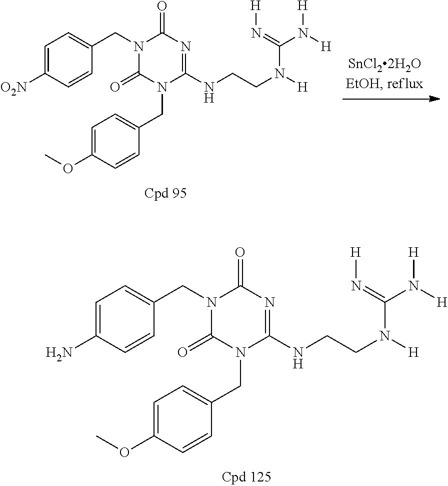

A mixture of the crude Compound 95 (39 mg, 0.083 mmol) and tin (II) chloride dihydrate (94 mg, 0.42 mmol) in 20 mL of EtOH was heated to reflux for 24 h. The solution was concentrated and the residue was purified by HPLC to give the title compound 125 as its TFA salt (6.5 mg, 0.015 mmol). ¹H NMR (DMSO-d₆) δ 3.30 (m, 4H), 3.73 (s, 3H), 4.80 (s, 2H), 4.98 (s, 2H), 6.56-6.78 (m, 2H), 6.88-6.91 (d, 2H, J=8.6 Hz), 7.13-7.20 (m, 4H), 7.43-7.47 (m, 1H), 7.92-7.99 (m, 1H).

Using the procedures of Example 9 and the appropriate reagents, starting materials and purification methods known to those skilled in the art, the following compounds of the present invention were prepared: compound 96.

### Example 10

### 3-(3,4-Dichloro-benzyl)-6-[2-(2-imino-imidazolidin-1-yl)-ethylamino]-1-(4-methoxy-benzyl)-1H-[1,3,5]triazine-2,4-dione (Cpd 60)

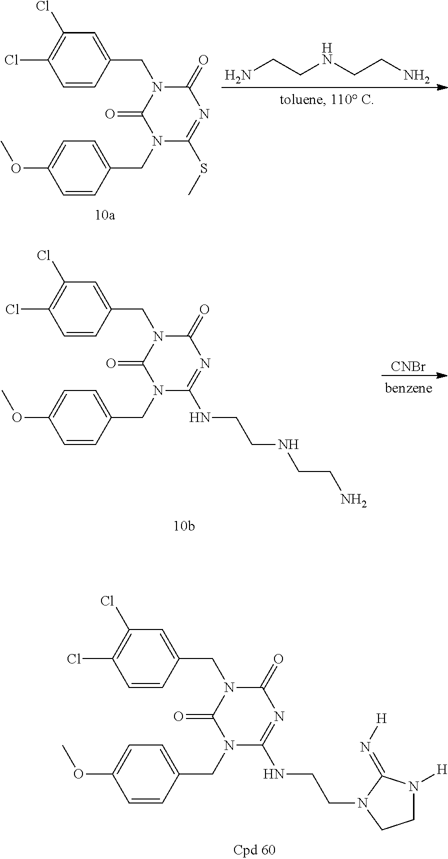

A. Compound 10a was prepared according to the methods described in Example 1, Step C, and substituting (3,4-dichloro-phenyl)-methanol for 4-ethylbenzyl alcohol.

B. 6-[2-(2-Amino-ethylamino)-ethylamino]-3-(3,4-dichloro-benzyl)-1-(4-methoxy-benzyl)-1H-[1,3,5]triazine-2,4-dione (Cpd 10b). To compound 10a (0.400 g, 0.968 mmol) in toluene (6 mL) was added 2,2′-diaminodiethylamine (0.300 g, 2.9 mmol) and the reaction mixture was heated at 110° C. for 4 h. The reaction mixture was cooled to room temperature and then water was added. The mixture was extracted with ethyl acetate, dried over sodium sulfate, filtered, and concentrated to give compound 10b (0.46 g) which was used in the subsequent reaction without further purification.

C. 3-(3,4-Dichloro-benzyl)-6-[2-(2-imino-imidazolidin-1-yl)-ethylamino]-1-(4-methoxy-benzyl)-1H-[1,3,5]triazine-2,4-dione. (Cpd 60). To compound 10b (0.100 g, 0.203 mmol) in benzene (2 mL) was added cyanogen bromide (0.022 g, 0.203 mmol). The reaction mixture was stirred for 2.5 h at room temperature. The reaction mixture was concentrated and then dissolved in a mixture of acetonitrile and methanol. The mixture was purified by reverse-phase chromatography to yield the title compound 60 (0.017 g). ¹H NMR (DMSO-d₆) δ 3.28-3.59 (8H, m), 3.66 (3H, s), 4.83 (2H, s), 4.92 (2H, s), 6.81-6.84 (2H, d, J =8.7 Hz), 7.09-7.12 (2H, d, 8.7 Hz), 7.19-7.22 (1H, d, J =8.3 Hz), 7.46 (1H,s), 7.51-7-54 (1H, d, J=8.3 Hz), 7.86-7.95 (3H, m).

### Example 11

### N-{2-[1-(4-Hydroxy-benzyl)-5-(4-methoxy-benzyl)-4,6-dioxo-1,4,5,6-tetrahydro-[1,3,5]triazin-2-ylamino]-ethyl}-guanidine (Cpd 143)

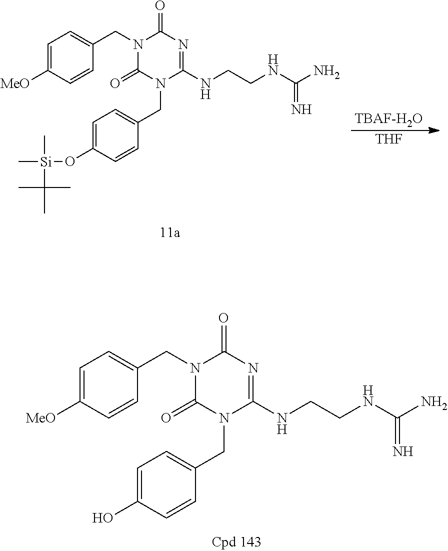

A. Compound 11a (50 mg, 0.09 mmol) was prepared according to the methods described in Example 2, and substituting [4-(tert-butyl-dimethyl-silanyloxy)-phenyl]-methanol for 4-methoxybenzyl alcohol in Step D.

B. Compound 11a was suspended in THF (2 mL) and the reaction mixture was treated with tetrabutylammonium fluoride monohydrate (24 mg, 0.09 mmol). The solution was stirred at room temperature overnight. The mixture was then concentrated under nitrogen and the residue was purified by reverse phase preparative HPLC to give the title compound 143 (3.8 mg) as a white solid. M+ (ES+)=440.1; ¹H NMR (MeOD, d₄). δ 3.32 (m, 2H), 3.50 (t, 2H, J=7.08 Hz), 3.78 (s, 3H), 4.99 (s, 2H), 5.03 (s, 2H), 6.77 (d, 2H, J=8.58 Hz), 6.85 (d, 2H, J=8.71 Hz), 7.07 (d, 2H, J=8.62 Hz), 7.36 (d, 2H, J=8.67 Hz).

### Example 12

### N-{2-[1-(4-Amino-benzyl)-5-(4-chloro-benzyl)-4,6-dioxo-1,4,5,6-tetrahydro-[1,3,5]triazin-2-ylamino]-ethyl}-guanidine (Cpd 122)

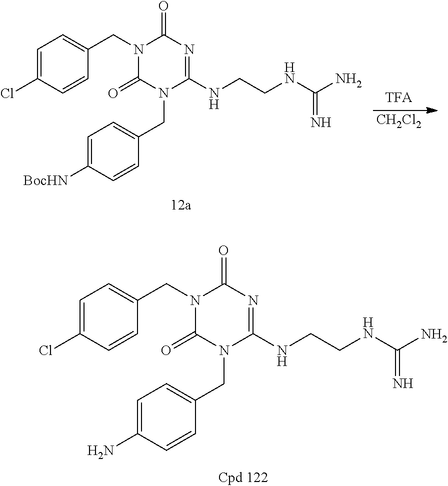

A. Compound 12a (50 mg, 0.09 mmol) was prepared according to the methods described in Example 2, and substituting (4-hydroxymethyl-phenyl)-carbamic acid tert-butyl ester for 4-methoxybenzyl alcohol in Step D.

B. Compound 12a (70 mg, 0.129 mmol) was suspended in dichloromethane (3 mL) and the solution was treated with trifluoroacetic acid (0.5 mL). The reaction was allowed to stir overnight at room temperature. The mixture was concentrated under nitrogen and the residue was purified by reverse phase preparative HPLC to give the title compound 122 (35.9 mg) as a white solid. M+ (ES+)=443.1; ¹H NMR (DMSO, d₆). δ 3.18-3.25 (m, 2H), 3.28-3.31 (m, 2H), 4.76 (s, 2H), 4.82 (s, 2H), 4.88 (s, 2H), 6.75 (d, 2H, J=8.25 Hz), 7.02 (d, 2H, J=8.38 Hz), 7.22-7.32 (m, 4H), 7.53 (d, 2H, J=4.02 Hz), 7.95 (m, 1H).

### Example 13

### N-{2-[5-(3,4-Dichloro-benzyl)-1-(4-methoxy-benzyl)-4,6-dioxo-1,4,5,6-tetrahydro-[1,3,5]triazin-2-ylamino]-ethyl}—N′-cyano-guanidine (Cpd 28)

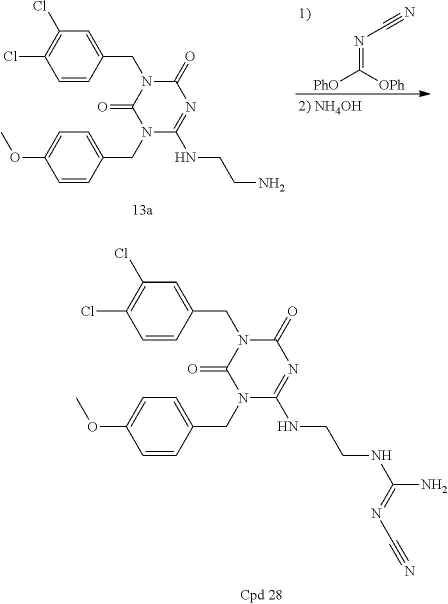

A. Compound 13a was prepared according to Example 1, substituting 3,4-dichlorophenyl methanol for 4-ethylbenzyl alcohol in Step D.

B. To a mixture of Cpd 13a (0.050 g, 0.11 mmol) in isopropyl alcohol (1 mL) was added triethylamine (0.017 mL, 0.12 mmol) and diphenyl N-cyanocarbonimidate (0.029 g, 0.12 mmol). The reaction mixture was stirred for 2 h at room temperature then concentrated under vacuum. The resulting residue was suspended in EtOH (0.75 mL) and NH₄OH (0.25 mL, 14.8 N (aq)) was added. The reaction mixture was stirred for 16 h at 50° C., concentrated under vacuum, and the resulting residue was purified by reverse-phase liquid chromatography using a gradient of 90:10 (water:acetonitrile, with 0.1% TFA) to 90:10 (acetonitrile:water, with 0.1% TFA) to give the title compound 28 (99% pure by HPLC, 0.0017 g); HRMS calcd. for C₂₂H₂₃Cl₂N₈O₃ m/z 517.1270 (M+H), found: 517.1281.

Using the procedures of Example 13 and the appropriate reagents, starting materials and purification methods known to those skilled in the art, the following compounds of the present invention were prepared: compound 143.

### Example 14

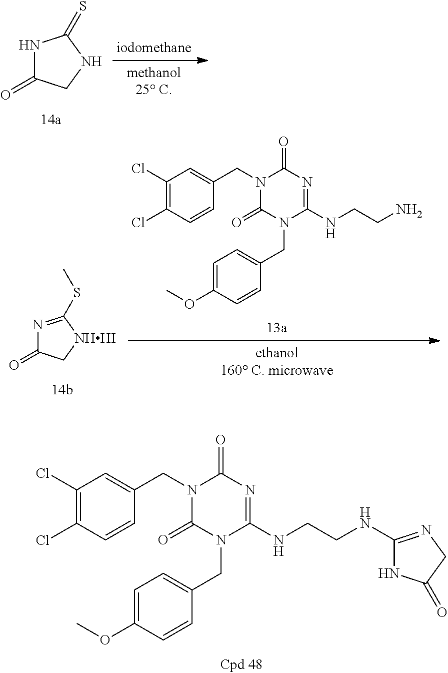

A. 1,5-Dihydro-2-(methylthio)-4H-imidazol-4-one monohydriodide (Cpd 15b). To a solution of compound 14a (420 mg, 3.6 mmol) in EtOH (5 mL) was added iodomethane (0.268 mL, 4.3 mmol). The mixture was stirred at 25° C. for 16 h, then concentrated to a residue to provide compound 14b, which was used in the next reaction without further purification.

B. 3-(3,4-Dichloro-benzyl)-1-(4-methoxy-benzyl)-6-[2-(5-oxo-4,5-dihydro-1H-imidazol-2-ylamino)-ethylamino]-1H-[1,3,5]triazine-2,4-dione 4 (Cpd 52). To a solution of compound 14b (0.0373 mg, 0.14 mmol) in ethanol (0.75 mL) was added compound 13a (50 mg, 0.13 mmol). The mixture was irradiated (μwave) at 160° C. for 15 min, concentrated, and the resulting residue was purified by reverse-phase liquid chromatography using a gradient of 90:10 (water:acetonitrile, with 0.1% TFA) to 90:10 (acetonitrile:water, with 0.1% TFA) to give the title compound 48 (89% pure by HPLC, 0.0025 g). HRMS calcd. for C₂₃H₂₄Cl₂N₂O₄ m/z 532.1267 (M+H), found: 532.1257.

### Example 15

### 3-(3,4-Dichloro-benzyl)-6-[2-(4,5-dihydro-1H-imidazol-2-ylamino)-ethylamino]-1-(4-methoxy-benzyl)-1H-[1,3,5]triazine-2,4-dione (Cpd 49)

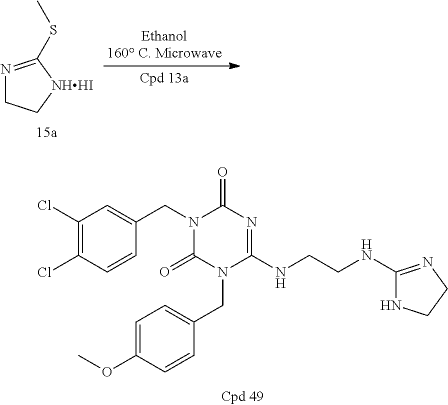

To a solution of compound 15a (0.054 mg, 0.22 mmol) in ethanol (1 mL) was added compound 13a (50 mg, 0.11 mmol). The mixture was irradiated in a microwave reactor at 160° C. for 15 min, concentrated, and the resulting residue was purified by reverse-phase liquid chromatography using a gradient of 90:10 (water:acetonitrile, with 0.1% TFA) to 90:10 (acetonitrile:water, with 0.1% TFA) to give the title compound 49 (93% pure by HPLC, 0.0082 g). HRMS calcd. for C₂₃H₂₆Cl₂N₇O₃ m/z 518.1474 (M+H), found: 518.1479.

### Example 16

### N-{2-[5-(4-Fluoro-benzyl)-1-(4-methoxy-benzyl)-4,6-dioxo-1,4,5,6-tetrahydro-[1,3,5]triazin-2-ylamino]-ethyl}—N-amino-guanidine (Cpd 93)

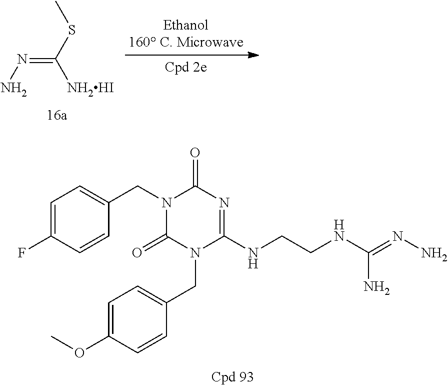

To a solution of compound 16a (0.061 mg, 0.22 mmol) in ethanol (1 mL) was added compound 2e (50 mg, 0.13 mmol). The mixture was irradiated in a microwave reactor at 160° C. for 15 min, concentrated, and the resulting residue was purified by reverse-phase liquid chromatography using a gradient of 90:10 (water:acetonitrile, with 0.1% TFA) to 90:10 (acetonitrile:water, with 0.1% TFA) to give the title compound 93 (99% pure by HPLC, 0.018 g). ¹H NMR (CDCl₃) δ 3.22-3.73 (2H, m), 3.38-3.55 (2H, m), 3.75 (2H, t, J=5.8 Hz), 3.77 (3H, s), 5.01 (2H, s), 5.07 (2H, s), 5.44-4.86 (2H, bs), 6.83 (2H, d, J=8.7 Hz), 6.90-7.03 (2H, m), 7.16 (2H, d, J=8.7 Hz), 7.48-7.36 (2H, m). HRMS calcd. for C₂₁H₂₆FN₈O₃ m/z 457.2112 (M+H), found: 457.2101.

### Example 17

### N-{2-[5-(4-fluoro-benzyl)-1-(4-methoxy-benzyl)-4,6-dioxo-1,4,5,6-tetrahydro-[1,3,5]triazin-2-ylamino]-acetyl}—N′-boc-guanidine (Cpd 132)

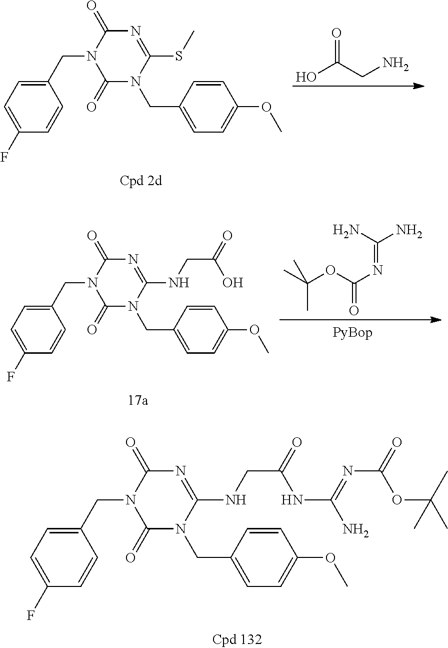

A. [5-(4-Fluoro-benzyl)-1-(4-methoxy-benzyl)-4,6-dioxo-1,4,5,6-tetrahydro-[1,3,5]triazin-2-ylamino]-acetic acid (Cpd 17a). To a solution of compound 2d (0.10 g, 0.26 mmol) in ethanol (1 mL) was added glycine (0.056 g, 0.75 mmol) and DIEA (0.143 mL, 0.82 mmol). The mixture was irradiated in a microwave reactor at 150° C. for 30 min then cooled to rt. Glycine (0.056 g, 0.75 mmol) and DIEA (0.143 mL, 0.82 mmol) were again added and the resulting mixture was irradiated (μwave) at 150° C. for 30 min, cooled to rt, concentrated, and the resulting residue was purified by reverse-phase liquid chromatography using a gradient of 90:10 (water:acetonitrile, with 0.1% TFA) to 90:10 (acetonitrile:water, with 0.1% TFA) to give compound 17a (99% pure by HPLC, 0.058 g). MS calcd. for C₂₀H₂₀FN₄O₅ m/z 415.1 (M+H), found: 415.1.

B. N-{2-[5-(4-fluoro-benzyl)-1-(4-methoxy-benzyl)-4,6-dioxo-1,4,5,6-tetrahydro-[1,3,5]triazin-2-ylamino]-acetyl}—N′-boc-guanidine (Cpd 132). To a solution of compound 17a (0.025 g, 0.047 mmol), DIEA (0.032 mL, 0.18 mmol), and monobocguanidine (0.015 g, 0.091 mmol) in DMF (0.40 mL) was added PyBop (0.047 g, 0.091 mmol). The mixture was stirred for 16 h at rt, quenched with water (3 mL), and the resulting solution was extracted 4×1 mL EtOAc. The combined organic layers were dried over Na₂SO₄, concentrated, and the resulting residue was purified by normal-phase flash chromatography on silica gel using a gradient of 50:50 (EtOAc:Heptane, with 0.1% Et₃N) to EtOAc (with 0.1% Et₃N) to give the title compound 132 (85% pure by HPLC, 0.0263 g). ¹H NMR (CDCl₃) 1.46 (9H, s), 3.79 (3H, s), 4.05 (2H, s), 5.07 (4H, s), 6.90 (2H, d, J═8.7 Hz), 6.98 (2H, at, J═6.7 Hz), 7.30 (2H, d, J═8.7 Hz), 7.50 (2H, dd, J═8.7 and 8.6 Hz), 8.61 (1H, bs); MS calcd. for C₂₆H₃₁FN₇O₆ m/z 556.2320 (M+H), found: 556.2341.

### Example 18

### N-{3-[1,3-Bis-(4-methoxy-benzyl)-2,6-dioxo-1,2,3,6-tetrahydro-pyrimidin-4-yl]-propyl}guanidine (Cpd 160)

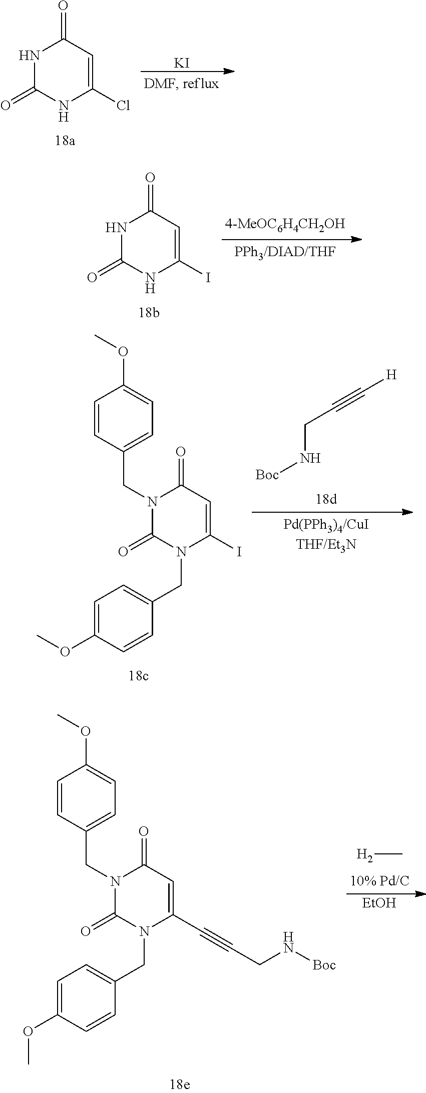

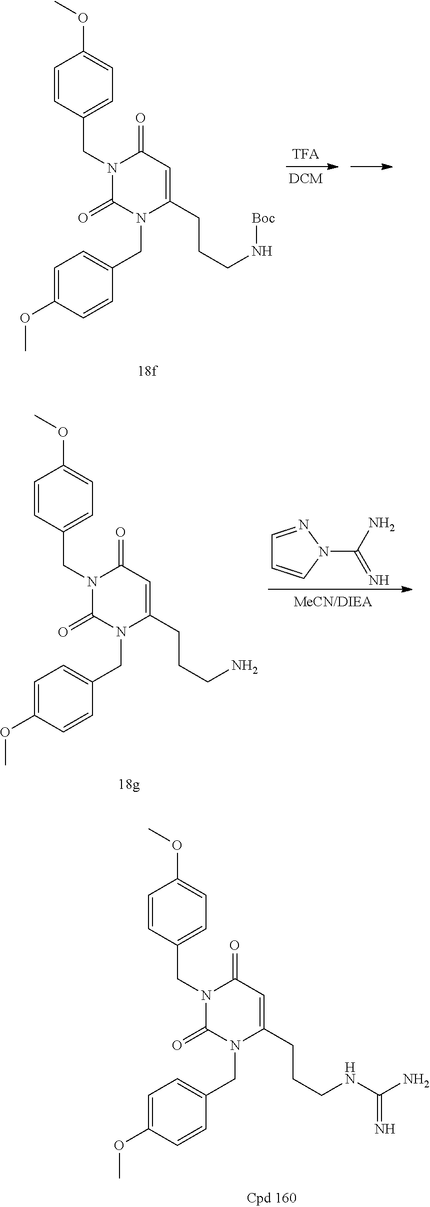

A. 6-Iodo-1H-pyrimidine-2,4-dione (18b). Compound 18a (5 g, 34 mmol) and sodium iodide (20 g) were dissolved in anhydrous DMF (50 mL) and heated to reflux for 1.5 h (Ar atmosphere). The DMF was evaporated, and the solid residue dissolved in H₂O (200 mL). The solution was stirred at RT for 4 h, a solid material was collected by vacuum filtration, and the solid was washed with H₂O and dried. The solid was crystallized from EtOAc, providing compound 18b. ¹H NMR (DMSO-d₆) δ 6.03 (s, 1H), 11.2 (s, 1H), 11.6 (s, 1H).

B. 6-Iodo-1,3-bis-(4-methoxy-benzyl)-1H-pyrimidine-2,4-dione (Cpd 18c). Compound 18b (1.00 g, 4.2 mmol), 4-methoxybenzyl alcohol (1.7 g, 3 eq), PPh₃ (4.00 g) were dissolved in dry THF (25 mL) under an atmosphere of N₂. DIAD was added dropwise at approximately 1 mL/min until the yellow color remained (about 4 eq total). The reaction mixture was stirred for 4 h at RT and evaporated. The residue was subjected to normal phase column chromatography (silica gel, gradient mixture heptane-ethyl acetate), providing compound 18c. ¹H NMR (CDCl₃) δ 3.78 (s, 3H), 3.79 (s, 3H), 5.04 (s, 2H), 5.27 (s, 2H), 6.54 (s, 1H), 6.82 (d, J═7.3 Hz, 2H), 6.86 (d, J═8.7 Hz, 2H), 7.22 (d, J═7.3 Hz, 2H), 7.42 (d, J═8.7 Hz, 2H). MS m/z 479.1 (M+H).

C. N-Boc-Propargylamine (Cpd 18d). Propargylamine (5.50 g, 0.1 mol) and di-tert-butyl dicarbonate (4.36 g, 2 eq.) were suspended together in 100 mL of a 10% aqueous solution of NaHCO₃. Reaction mixture was stirred overnight and extracted by EtOAc (3×20 mL). The organic phases were combined together, washed with citric acid 10% aq., dried over MgSO₄, filtered and evaporated, providing compound 18d as white solid (10.1 g, 65% yield). ¹H N MR (CDCl₃) δ 4.72 (bs, 1H), 3.91 (d, J═3.0 Hz, 2H), 2.22 (t, J═2.9 Hz, 1H), 1.51 (s, 9H).

D. {3-[1,3-Bis-(4-methoxy-benzyl)-2,6-dioxo-1,2,3,6-tetrahydro-pyrimidin-4-yl]-prop-2-ynyl}-carbamic acid tert-butyl ester (Cpd 18e). Compound 18c (240 mg, 0.5 mmol) and compound 18d (150 mg, 1 mmol) were dissolved in a mixture of dry THF (10 mL) and Et₃N (2 mL). Pd(PPh₃)₄ (40 mg) and copper (I) iodide (20 mg) were added simultaneously in one portion. The reaction mixture was stirred overnight at RT under a N₂ atmosphere and evaporated. The residue was subjected to normal phase column chromatography (silica gel column, heptane-EtOAc 8:2 to 0:10 gradient mixture), providing compound 18e as yellow solid. ¹H NMR (CDCl₃) δ 7.42 (d, J═8.7 Hz, 2H), 7.28 (d, J═8.7 Hz, 2H), 6.84 (d, J═9.1 Hz, 2H), 6.81 (d, J═9.1 Hz, 2H), 5.93 (s, 1H), 5.08 (s, 2H), 5.03 (s, 2H), 3.78 (s, 3H), 3.76 (s, 3H), 1.44 (s, 9H).

E. {3-[1,3-Bis-(4-methoxy-benzyl)-2,6-dioxo-1,2,3,6-tetrahydro-pyrimidin-4-yl]-propyl}-carbamic acid tert-butyl ester (Cpd 18f). Compound 18e (500 mg, 0.1 mmol) was dissolved in EtOH (10 mL) and suspended with 10% Pd on carbon (40 mg). The reaction mixture was hydrogenated for 24 h at RT under atmospheric pressure, filtered through a diatomaceous earth plug, and evaporated, providing 501 mg of white solid 18f. ¹H NMR (CDCl₃) δ 7.38 (d, J═8.7 Hz, 2H), 7.00 (d, J═8.7 Hz, 2H), 6.87-6.72 (m, 4H), 5.54 (s, 1H), 5.01 (s, 2H), 4.99 (s, 2H), 3.71 (s, 3H), 3.70 (s, 3H), 3.08-3.00 (m, 2H), 2.39-2.30 (m, 2H), 1.65-1.55 (m, 2H), 1.34 (s, 9H).

F. 6-(3-Amino-propyl)-1,3-bis-(4-methoxy-benzyl)-1H-pyrimidine-2,4-dione (Cpd 18g). Compound (18f) (500 mg, 0.098 mmol) was dissolved in 10 ml DCM-TFA 9:1 mixture and stirred at RT. Reaction was monitored by HPLC. After 10 h all starting material disappeared, reaction mixture was filtered through a diatomaceous earth plug and evaporated, providing 350 mg of 18g (TFA salt, white solid). MS m/z 410.0 (M+H).

G. N-{3-[1,3-Bis-(4-methoxy-benzyl)-2,6-dioxo-1,2,3,6-tetrahydro-pyrimidin-4-yl]-propyl}-guanidine (Cpd 160). Compound 18g (260 mg TFA salt, 0.5 mmol) and 1H-pyrazole-1-carboxamidine hydrochloride (290 mg, 4 eq) were suspended in 20 ml MeCN-DIEA 9:1 mixture, stirred at RT overnight and evaporated. The residue was dissolved in MeOH and subjected to HPLC, providing after lyophilization 128.5 mg of Compound 160 (30% yield, white powder, di-TFA salt). ¹H NMR (CD₃CN) δ 7.50 (m, 1H), 7.28 (d, J═8.7 Hz, 2H), 7.08 (d, J═8.7 Hz, 2H), 6.87 (d, J═7.6 Hz, 2H), 6.83 (d, J═7.7 Hz, 2H), 6.6 (bs, 3H), 5.61 (s, 1H), 5.01 (s, 2H), 4.99 (s, 2H), 3.75 (s, 6H), 3.14-3.07 (m, 2H), 2.55-2.45 (m, 2H), 1.79-1.69 (m, 2H). MS m/z 452.0 (M+H).

### Example 19

### N-{2-[1,3-Bis-(4-methoxy-benzyl)-2,6-dioxo-1,2,3,6-tetrahydro-pyrimidin-4-yloxy]-ethyl}-guanidine (Cpd 158)

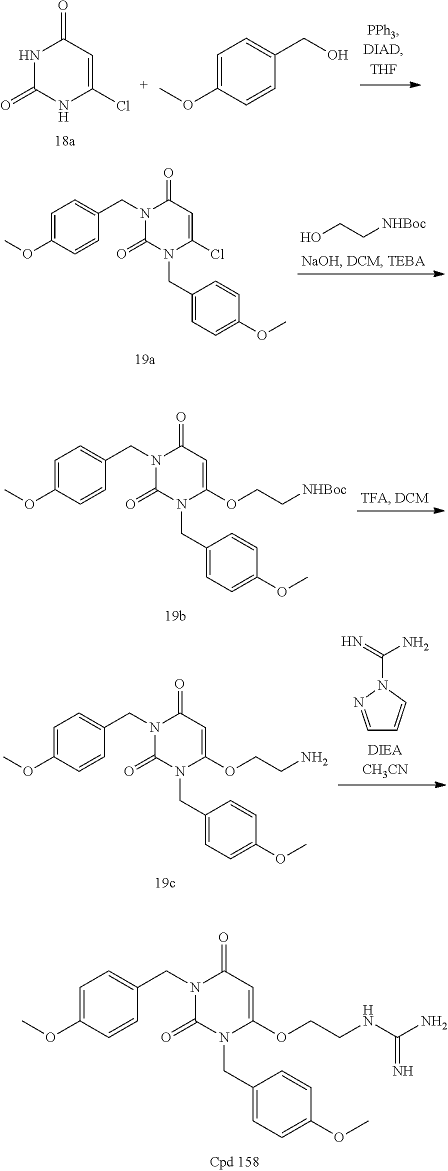

A. 6-Chloro-1,3-bis-(4-methoxy-benzyl)-1H-pyrimidine-2,4-dione (Cpd 19a). A solution of compound 18a (500 mg, 3.4 mmol), 4-methoxybenzyl alcohol (990 mg, 7.2 mmol), triphenylphosphine (2.9 g, 11.2 mmol), and diisopropylazodicarboxylate (1.6 mL, 8.2 mmol) in THF (100 mL) was allowed to stir at room temperature overnight. The solution was concentrated. The concentrate was taken up in ethyl acetate and washed sequentially with saturated sodium bicarbonate and brine. The organic layer was dried over magnesium sulfate, filtered, and the filtrate was concentrated. The concentrate was purified by reverse phase chromatography to give the title compound 19a (552 mg). M+ (ES+)═386.9. ¹H NMR (methanol-d₄). δ 3.75 (s, 3H), 3.76 (s, 3H), 5.01 (s, 2H), 5.21 (s, 2H), 5.99 (s, 1H), 6.83 (d, 4H, J═8.9 Hz), 6.87 (d, 2H, J═8.9 Hz), 7.23 (d, 2H, 8.5 Hz), 7.32 (d, 2H, J═8.9 Hz).

B. {2-[1,3-Bis-(4-methoxy-benzyl)-2,6-dioxo-1,2,3,6-tetrahydro-pyrimidin-4-yloxy]-ethyl}-carbamic acid tert-butyl ester (Cpd 19b). To a solution of t-butyl-N-(2-hydroxyethyl)carbamate (40 μL, 0.26 mmol), benzyltriethyammonium chloride (3 mg, 0.013 mmol) and 3M NaOH solution (870 μL, 2.6 mmol) was added a solution of compound 19a (50 mg, 0.13 mmol) in dichloromethane (3 mL). After stirring overnight, the mixture was separated. The aqueous layer was extracted two times with dichloromethane. The combined organic extracts were dried over magnesium sulfate, filtered, and the filtrate was concentrated. The concentrate was purified by reverse phase chromatography after dissolving in DMSO to afford the title compound 19b as white powder. M+ (ES+)═512.0. ¹H NMR (DMSO, d₆). δ 1.36 (s, 9H), 3.33 (m, 2H), 3.72 (m, 2H), 4.88 (s, 2H), 4.94 (s, 2H), 6.85 (m, 4H), 7.20 (m, 4H).

C. 6-(2-Amino-ethoxy)-1,3-bis-(4-methoxy-benzyl)-1H-pyrimidine-2,4-dione (Cpd 19c). To a solution of compound 19b (assume 0.12 mmol) in dichloromethane (2 mL) was added trifluoroacetic acid (50 μL). Additional TFA (100 μL) was added. Additional TFA (150 μL) was added and the reaction was allowed to stir for an additional 16 hrs. The mixture was concentrated and purified by reverse phase chromatography to obtain the title compound 19c (24 mg) as a white solid. M+ (ES+)═411.9. ¹H NMR (methanol-d₄). δ 3.36 (t, 2H, J═4.9, 5.0 Hz), 3.75 (s, H), 3.76 (s, 3H), 5.01 (s, 2H), 5.10 (s, 2H), 5.28 (s, 1H), 6.84 (m, 4H), 7.22 (d, 2H, J═8.6 Hz), 7.30 (d, 2H, J═5.6 Hz).

D. N-{2-[1,3-Bis-(4-methoxy-benzyl)-2,6-dioxo-1,2,3,6-tetrahydro-pyrimidin-4-yloxy]-ethyl}-guanidine (Cpd 158). A mixture of compound 19c (20 mg, 0.05 mmol), 1H-pyrazole-1-carboxamidine HCl (8.7 mg, 0.06 mmol), and DIEA (16.5 μL, 0.15 mmol) in acetonitrile (5 mL) was allowed to stir at rt overnight. The mixture was concentrated and purified by reverse phase chromatography to obtain the title compound 158 as a white solid. M+ (ES+)═453.9. ¹H NMR (DMSO, d₆). δ 3.57 (t, 2H, J═4.7, 5.2 Hz), 3.71 (s, 3H), 3.72 (s, 3H), 4.20 (t, 2H, J═4.9, 4.6 Hz), 4.89 (s, 2H), 4.94 (s, 2H), 5.31 (s, 1H), 6.87 (m, 4H), 7.22 (m, 4H), 7.78 (t, 1H, J═5.6, 5.6 Hz).

### Example 20

### N-{2-[1,3-Bis-(4-methoxy-benzyl)-2,6-dioxo-1,2,3,6-tetrahydro-pyrimidin-4-ylsulfanyl]-ethyl}-guanidine (Cpd 159)

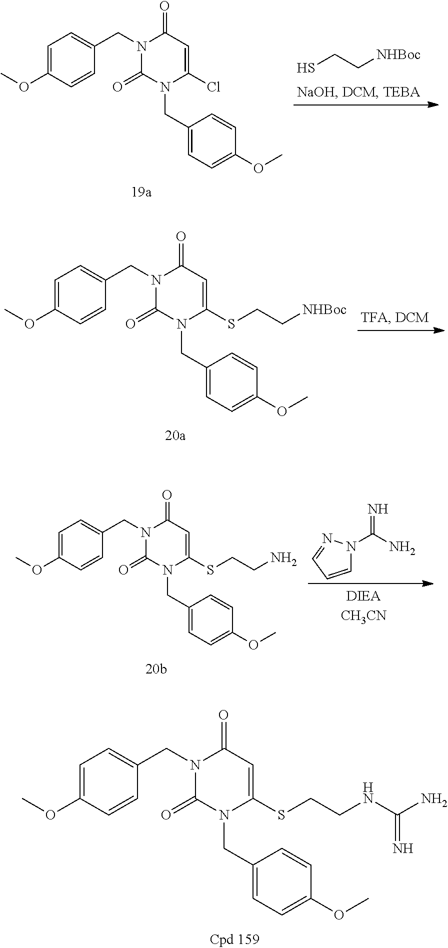

A. {2-[1,3-Bis-(4-methoxy-benzyl)-2,6-dioxo-1,2,3,6-tetrahydro-pyrimidin-4-ylsulfanyl]-ethyl}-carbamic acid tert-butyl ester (Cpd 20a). To a solution of 2-(boc-amino)ethanethiol (87 μL, 0.52 mmol), 3M NaOH (1.7 mL, 5.2 mmol), and benzyltriethyammonium chloride (5 mL) was added a mixture of compound 19a (100 mg, 0.26 mmol) in dichloromethane (5 mL). The mixture was allowed to stir overnight at rt. The mixture was separated, and the aqueous layer was washed with dichloromethane. The combined organic extracts were dried over magnesium sulfate, filtered, and the filtrate was concentrated. The concentrate was triturated in MeOH and collected to obtain the title compound 20a as a white solid. M+ (ES+)═527.8.

B. 6-(2-Amino-ethylsulfanyl)-1,3-bis-(4-methoxy-benzyl)-1H-pyrimidine-2,4-dione (Cpd 20b). To a mixture of compound 20a (78 mg, 0.15 mmol) in dichloromethane (3 mL) was added TFA (0.5 mL), and the reaction was stirred for 2 h. The mixture was concentrated and the residue was purified by reverse phase chromatography to obtain the title compound 20b as a white powder. M+ (ES+)═427.8. ¹H NMR (methanol-d₄). δ 3.37 (s, 6H), 4.84 (m, 4H), 5.05 (s, 2H), 5.20 (s, 2H), 6.85 (m, 4H), 7.18 (d, 2H, J═8.7 Hz), 7.34 (d, 2H, J═6.6 Hz).

C. N-{2-[1,3-Bis-(4-methoxy-benzyl)-2,6-dioxo-1,2,3,6-tetrahydro-pyrimidin-4-ylsulfanyl]-ethyl}-guanidine (Cpd 159). A solution of compound 20b (assumed 0.09 mmol), 1-H-pyrazole-1-carboxamidine HCl (16 mg, 0.108 mmol), and DIEA (5 μL, 0.45 mmol) in acetonitrile (3 mL) was allowed to stir at rt overnight. The mixture was concentrated and purified by reverse phase chromatography to obtain the title compound 159 as a white powder. M+ (ES+)═469.8. ¹H NMR (DMSO, d₆). δ 3.19 (t, 2H, J═6.2, 6.6 Hz), 3.42 (m, 2H), 3.72 (s, 6H), 4.93 (s, 2H), 5.08 (s, 2H), 5.84 (s, 1H), 6.86 (d, 2H, J═8.7 Hz), 6.90 (s, 2H, J═8.7 Hz), 7.16 (d, 2H, J═8.7 Hz), 7.25 (d, 2H, J═8.6 Hz), 7.60 (m, 1H).

### Example 21

### 1,3-Bis-(4-methoxy-benzyl)-2,6-dioxo-1,2,3,6-tetrahydro-pyrimidine-4-carboxylic acid (2-guanidino-ethyl)-amide (Cpd 157)

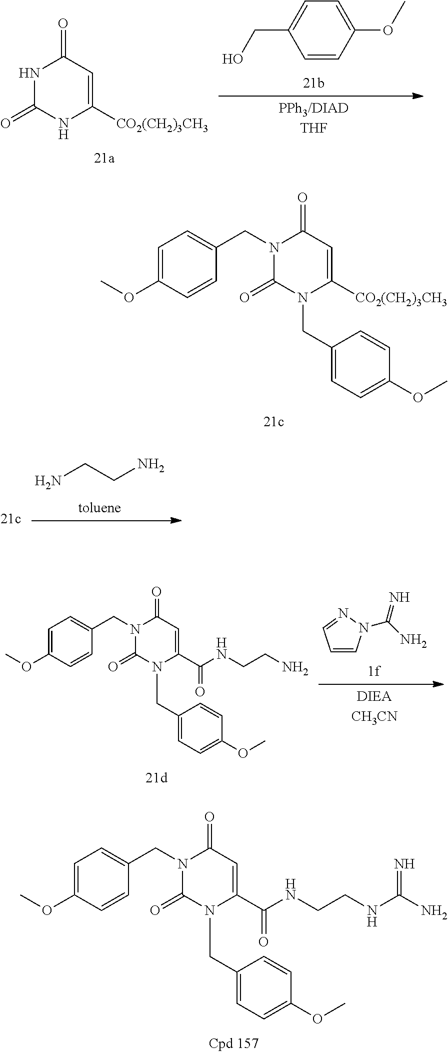

A. 1,3-Bis-(4-methoxy-benzyl)-2,6-dioxo-1,2,3,6-tetrahydro-pyrimidine-4-carboxylic acid butyl ester (Cpd 21c). A mixture Compound 21a (1.00 g, 4.7 mmol), 4-methoxybenzyl alcohol (Cpd 21b, 2.00 g, 14.1 mmol) and PPh₃ (5.00 g, 19 mmol) were dissolved in 50 mL of dry THF at 20° C. DIAD (3.8 g, 18 mmol) was added dropwise, and the reaction mixture was allowed to stir overnight at room temperature. The reaction mixture was washed with water, and extracted with EtOAc. The combined organic fractions were dried over MgSO₄, filtered and evaporated, providing compound 21c as white solid. M+ (ES+)═453.3. ¹H NMR (CDCl₃). δ 7.43 (d, 2H, J═8.7 Hz), 7.07 (d, 2H, J═8.7 Hz), 6.88-6.78 (m, 4H), 6.08 (s, 1H), 5.27 (s, 2H), 5.09 (s, 2H), 4.13 (t, 3H, J═6.6 Hz), 3.79 (s, 3H), 3.77 (s, 3H), 1.60-1.48 (m, 2H), 1.35-1.20 (m, 2H), 0.90 (t, 3H, J═7.2 Hz).

B. 1,3-Bis-(4-methoxy-benzyl)-2,6-dioxo-1,2,3,6-tetrahydro-pyrimidine-4-carboxylic acid (2-amino-ethyl)-amide (Cpd 21d). Compound 21c (390 mg, 0.86 mmol) and ethylene diamine (400 μL, 6 mmol) in 10 mL of toluene were refluxed for 4 hrs, cooled to rt, and concentrated under reduced pressure. The resultant residue was subjected to HPLC to give the di-TFA salt of 21d.

C. 1,3-Bis-(4-methoxy-benzyl)-2,6-dioxo-1,2,3,6-tetrahydro-pyrimidine-4-carboxylic acid (2-guanidino-ethyl)-amide (Cpd 157). The di-TFA salt of 21d (280 mg, 0.42 mmol) was dissolved in a mixture of 5 mL of MeCN and 1 mL of DIEA. Compound 1f (200 mg, 1.8 mmol) was added as one portion, the reaction mixture was allowed to stir overnight at room temperature, and then concentrated under reduced pressure. The resultant residue was subjected to HPLC, providing 59.4 mg of the di-TFA salt of Cpd 157. M+ (ES+)═481.2. ¹H NMR (DMSO, d₆). δ 7.21 (d, 2H, J═8.6 Hz), 7.16 (d, 2H, J═8.6 Hz), 6.85 (d, 4H, J═8.7 Hz), 6.69 (s, 1H), 5.99 (s, 1H), 4.87 (s, 2H), 4.92 (s, 2H), 3.72 (s, 6H), 3.65-3.50 (m, 2H), 3.24 (broad s, 4H), 3.05-3.15 (m, 2H).

Compounds 1 through 160 of Formula (I) in Table 1 were synthesized using the procedures described in the schemes and specific examples provided herein.

TABLE 1

| Cpd # | A₁ | L₁ | D | W | L₂ | Q |
|---|---|---|---|---|---|---|
| 1 | phenyl | —CH₂— | —CH₂-(4-fluoro- | N | —NH(CH₂)₂— | —NHC(═NH)NH₂ |
| | | | phenyl) | | | |
| 2 | phenyl | —CH₂— | —CH₂-(4-methoxy- | N | —NH(CH₂)₂— | —NHC(═NH)NH₂ |
| | | | phenyl) | | | |
| 3 | phenyl | —CH₂— | —CH₂-(4- | N | —NH(CH₂)₂— | —NHC(═NH)NH₂ |
| | | | methylcarboxy- | | | |
| | | | phenyl) | | | |
| 4 | phenyl | —(CH₂)₂— | —CH₂-(4-methoxy- | N | —NH(CH₂)₂— | —NHC(═NH)NH_{2′} |
| | | | phenyl) | | | |
| 5 | H | —(CH₂)₄— | —CH₂-(4-methoxy- | N | —NH(CH₂)₂— | —NHC(═NH)NH₂ |
| | | | phenyl) | | | |
| 6 | furan-2-yl | —CH₂— | —CH₂-(4-methoxy- | N | —NH(CH₂)₂— | —NHC(═NH)NH₂ |
| | | | phenyl) | | | |
| 7 | phenyl | —CH₂— | —CH₂-(3- | N | —NH(CH₂)₂— | —NHC(═NH)NH₂ |
| | | | trifluoromethyl- | | | |
| | | | phenyl) | | | |
| 8 | phenyl | —CH₂— | —CH₂-(4-t-butyl- | N | —NH(CH₂)₂— | —NHC(═NH)NH₂ |
| | | | phenyl) | | | |
| 9 | phenyl | —CH₂— | —CH₂-(4-nitro-phenyl) | N | —NH(CH₂)₂— | —NHC(═NH)NH₂ |
| 10 | phenyl | —CH₂— | —CH₂-(4-methoxy- | N | —NH(CH₂)₂— | —ONHC(═NH)NH₂ |
| | | | phenyl) | | | |
| 11 | phenyl | —CH₂— | —CH₂-pyridin-4-yl | N | —NH(CH₂)₂— | —NHC(═NH)NH₂ |
| 12 | phenyl | —CH₂— | —CH₂-(4-ethoxy- | N | —NH(CH₂)₂— | —NHC(═NH)NH₂ |
| | | | phenyl) | | | |
| 13 | phenyl | —CH₂— | —CH₂-(4- | N | —NH(CH₂)₂— | —NHC(═NH)NH₂ |
| | | | difluoromethoxy- | | | |
| | | | phenyl) | | | |
| 14 | phenyl | —CH₂— | —CH₂-(4-n-butyl- | N | —NH(CH₂)₂— | —NHC(═NH)NH₂ |
| | | | phenyl) | | | |
| 15 | phenyl | —CH₂— | —CH₂-(4- | N | —NH(CH₂)₂— | —NHC(═NH)NH₂ |
| | | | trifluoromethyl- | | | |
| | | | phenyl) | | | |
| 16 | 2-fluoro-phenyl | —CH₂— | —CH₂-(4-methoxy- | N | —NH(CH₂)₂— | —NHC(═NH)NH₂ |
| | | | phenyl) | | | |
| 17 | 4-fluoro-phenyl | —CH₂— | —CH₂-(4-methoxy- | N | —NH(CH₂)₂— | —NHC(═NH)NH₂ |
| | | | phenyl) | | | |
| 18 | 3,4-dichloro- | —CH₂— | —CH₂-(4-methoxy- | N | —NH(CH₂)₂— | —NHC(═NH)NH₂ |
| | phenyl | | phenyl) | | | |
| 19 | phenyl | —CH₂— | —CH₂-(4- | N | —NH(CH₂)₂— | —NHC(═NH)NH₂ |
| | | | trifluoromethoxy- | | | |
| | | | phenyl) | | | |
| 20 | 3-methoxy- | —CH₂— | —CH₂-(4-methoxy- | N | —NH(CH₂)₂— | —NHC(═NH)NH₂ |
| | phenyl | | phenyl) | | | |
| 21 | 2-methoxy- | —CH₂— | —CH₂-(4-methoxy- | N | —NH(CH₂)₂— | —NHC(═NH)NH₂ |
| | phenyl | | phenyl) | | | |
| 22 | phenyl | —CH₂— | —CH₂-(4- | N | —NH(CH₂)₂— | —NHC(═NH)NH₂ |
| | | | aminocarbonyl- | | | |
| | | | phenyl) | | | |
| 23 | phenyl | —CH₂— | —CH₂-(4- | N | —NH(CH₂)₂— | —NHC(═NH)NH₂ |
| | | | methylcarboxyl | | | |
| | | | amino-phenyl) | | | |
| 24 | 4-fluoro-phenyl | —CH₂— | —CH₂-(4-ethoxy- | N | —NH(CH₂)₂— | —NHC(═NH)NH₂ |
| | | | phenyl) | | | |
| 25 | phenyl | —(R,R— | —CH₂-(4-methoxy- | N | —NH(CH₂)₂— | —NHC(═NH)NH₂ |
| | | CH(CH₃) | phenyl) | | | |
| | | CH(CH₃))— | | | | |
| 26 | phenyl | —(R,R— | —CH₂-(4-methoxy- | N | —NH(CH₂)₂— | —NHC(═NH)NH₂ |
| | | CH(CH₃)CH | phenyl) | | | |
| | | (CH₃))— | | | | |
| 27 | 3,4-dichloro- | —CH₂— | —CH₂-(4-methoxy- | N | —NH(CH₂)₂— | —ONHC(═NH)NH₂ |
| | phenyl | | phenyl) | | | |
| 28 | 3,4-dichloro- | —CH₂— | —CH₂-(4-methoxy- | N | —NH(CH₂)₂— | —NHC(═N—CN)NH₂ |
| | phenyl | | phenyl) | | | |
| 29 | 3,4-dichloro- | —CH₂— | —CH₂-(4-ethoxy- | N | —NH(CH₂)₂— | —NHC(═NH)NH₂ |
| | phenyl | | phenyl) | | | |
| 30 | 4-chloro-phenyl | —CH₂— | —CH₂-(4-methoxy- | N | —NH(CH₂)₂— | —NHC(═NH)NH₂ |
| | | | phenyl) | | | |
| 31 | 4-methoxy- | —CH₂— | —CH₂-(4-methoxy- | N | —NH(CH₂)₂— | —NHC(═NH)NH₂ |
| | phenyl | | phenyl) | | | |
| 32 | 3,4-dichloro- | —CH₂— | —CH₂-(4-methoxy- | N | —NH(CH₂)₄— | —NHC(═NH)NH₂ |
| | phenyl | | phenyl) | | | |
| 33 | 4-fluoro-phenyl | —CH₂— | —(CH₂)₂-(4-methoxy- | N | —NH(CH₂)₂— | —NHC(═NH)NH₂ |
| | | | phenyl) | | | |
| 34 | 3,4-dichloro- | —CH₂— | —CH₂-(4-n-propyl- | N | —NH(CH₂)₂— | —NHC(═NH)NH₂ |
| | phenyl | | phenyl) | | | |
| 35 | 3,4-dichloro- | —CH₂— | —CH₂-(4-i-propyl- | N | —NH(CH₂)₂— | —NHC(═NH)NH₂ |
| | phenyl | | phenyl) | | | |
| 36 | 3,4-dichloro- | —CH₂— | —CH₂-(4- | N | —NH(CH₂)₂— | —NHC(═NH)NH₂ |
| | phenyl | | cyclopentyloxy- | | | |
| | | | phenyl) | | | |
| 37 | 3,4-dichloro- | —CH₂— | —CH₂-(4-methylthio- | N | —NH(CH₂)₂— | —NHC(═NH)NH₂ |
| | phenyl | | phenyl) | | | |
| 38 | 3,4-dichloro- | —CH₂— | —CH₂-(4-ethyl-phenyl) | N | —NH(CH₂)₂— | —NHC(═NH)NH₂ |
| | phenyl | | | | | |
| 39 | 3-chloro-phenyl | —CH₂— | —CH₂-(4-methoxy- | N | —NH(CH₂)₂— | —NHC(═NH)NH₂ |
| | | | phenyl) | | | |
| 40 | 3,4-dichloro- | —CH₂— | —CH₂-(4- | N | —NH(CH₂)₂— | —NHC(═NH)NH₂ |
| | phenyl | | trifluoromethoxy- | | | |
| | | | phenyl) | | | |
| 41 | 3,4-dichloro- | —CH₂— | —CH₂-(4- | N | —NH(CH₂)₂— | —NHC(═NH)NH₂ |
| | phenyl | | difluoromethoxy- | | | |
| | | | phenyl) | | | |
| 42 | 3,4-dichloro- | —CH₂— | —CH₂-(4-methoxy- | N | cis-racemic-1,2- | —NHC(═NH)NH₂ |
| | phenyl | | phenyl) | | cyclohexyl | |
| 43 | 3,4-dichloro- | —CH₂— | —CH₂-(4-methoxy- | N | trans (1S,2S)- | —NHC(═NH)NH₂ |
| | phenyl | | phenyl) | | cyclohexyl- | |
| 44 | 3,4-dichloro- | —CH₂— | —CH₂-(4-methoxy- | N | —NH(CH₂)₂— | —NHC(═NH)NH₂ |
| | phenyl | | phenyl) | | | |
| 45 | 4-methylthio- | —CH₂— | —CH₂-(4-methoxy- | N | —NH(CH₂)₂— | —NHC(═NH)NH₂ |
| | phenyl | | phenyl) | | | |
| 46 | 4-ethyl-phenyl | —CH₂— | —CH₂-(4-methoxy- | N | —NH(CH₂)₂— | —NHC(═NH)NH₂ |
| | | | phenyl) | | | |
| 47 | 3,4-dichloro- | —CH₂— | —CH₂-(4-methoxy- | N | trans(1R,2R)- | —NHC(═NH)NH₂ |
| | phenyl | | phenyl) | | cyclohexyl- | |
| 48 | 3,4-dichloro- | —CH₂— | —CH₂-(4-methoxy- | N | —NH(CH₂)₂— | —NH(3,5-dihydro- |
| | phenyl | | phenyl) | | | imidazol-4-on-2-yl) |
| 49 | 3,4-dichloro- | —CH₂— | —CH₂-(4-methoxy- | N | —NH(CH₂)₂— | —NH(4,5-dihydro- |
| | phenyl | | phenyl) | | | 1H-imidazol-2-yl) |
| 50 | 3,4-dichloro- | —CH₂— | —CH₂-(4- | N | —NH(CH₂)₂— | —NHC(═NH)NH₂ |
| | phenyl | | methylcarbonyl | | | |
| | | | amino-phenyl) | | | |
| 51 | 3,4-dichloro- | —CH₂— | —CH₂-(4- | N | —NH(CH₂)₂— | —NHC(═NH)NH₂ |
| | phenyl | | aminocarbonyl- | | | |
| | | | phenyl) | | | |
| 52 | 3,4-dichloro- | —CH₂— | —CH₂-(3-ethoxy- | N | —NH(CH₂)₂— | —NHC(═NH)NH₂ |
| | phenyl | | phenyl) | | | |
| 53 | 3,4-dichloro- | —CH₂— | —CH₂-(4-ethoxy- | N | —NH(CH₂)₂— | —NHC(═NH)NH- |
| | phenyl | | phenyl) | | | ethyl |
| 54 | 3,4-dichloro- | —CH₂— | —CH₂-(4-methoxy- | N | —NH(CH₂)₂— | —NHC(═NH)NH- |
| | phenyl | | phenyl) | | | propyl |
| 55 | 3,4-dichloro- | —CH₂— | —CH₂-(4-methoxy- | N | pyrrolindin-1-yl | 3-NHC(═NH)NH₂ |
| | phenyl | | phenyl) | | | |
| 56 | 4-chloro-phenyl | —CH₂— | —CH₂-(4-methoxy- | N | -trans (1R,2R)- | —NHC(═NH)NH₂ |
| | | | phenyl) | | cyclohexyl- | |
| 57 | 3,4-dichloro- | —CH₂— | —CH₂-(3- | N | —NH(CH₂)₂— | —NHC(═NH)NH₂ |
| | phenyl | | difluoromethoxy- | | | |
| | | | phenyl) | | | |
| 58 | 3,4-dichloro- | —CH₂— | —CH₂-(4-methoxy- | N | —NH(CH₂)₂— | —NHC(═NH)NH |
| | phenyl | | phenyl) | | | (i-propyl) |
| 59 | 3,4-dichloro- | —CH₂— | —CH₂-(4-methoxy- | N | —NH(CH₂)₂— | —N(ethyl) |
| | phenyl | | phenyl) | | | C(═NH)NH₂ |
| 60 | 3,4-dichloro- | —CH₂— | —CH₂-(4-methoxy- | N | —NH(CH₂)₂— | 2-imino- |
| | phenyl | | phenyl) | | | imidazolid-1-yl |
| 61 | 3,4-dichloro- | —CH₂— | —CH₂-(4-methoxy- | N | —NH(CH₂)₂— | —NHC(═NH)NH |
| | phenyl | | phenyl) | | | (n-butyl) |
| 62 | 3,4-dichloro- | —CH₂— | —CH₂-(4-methoxy- | N | —NH(CH₂)₂— | —NHC(═NH)NH |
| | phenyl | | phenyl) | | | (cyclohexyl) |
| 63 | 3,4-dichloro- | —CH₂— | —CH₂-(4-methoxy- | N | —NH(CH₂)₂— | —NHC(═NH)NH |
| | phenyl | | phenyl) | | | (benzyl) |
| 64 | 3,4-dichloro- | —CH₂— | —CH₂-(4-methoxy- | N | —NH(CH₂)₂— | —NHC(═NH)NH |
| | phenyl | | phenyl) | | | (tetrahydrofuran-2- |
| | | | | | | ylmethyl) |
| 65 | 3,4-dichloro- | —CH₂— | —CH₂-(4-methoxy- | N | —NH(CH₂)₂— | —NHC(═NH)NH |
| | phenyl | | phenyl) | | | (phenylethyl) |
| 66 | 3,4-dichloro- | —CH₂— | —CH₂-(4-methoxy- | N | —NH(CH₂)₂— | —NHC(═NH)NH |
| | phenyl | | phenyl) | | | (furan-2-ylmethyl) |
| 67 | 3,4-dichloro- | —CH₂— | —CH₂-(4-methoxy- | N | —NH(CH₂)₂— | —NHC(═NH)NH |
| | phenyl | | phenyl) | | | (2-methoxy-ethyl) |
| 68 | 3,4-dichloro- | —CH₂— | —CH₂-(4-methoxy- | N | —NH(CH₂)₃— | —NHC(═NH)NH₂ |
| | phenyl | | phenyl) | | | |
| 69 | 3,4-dichloro- | —CH₂— | —(CH₂)₆—H | N | —NH(CH₂)₃— | —NHC(═NH)NH₂ |
| | phenyl | | | | | |
| 70 | 3,4-dichloro- | —CH₂— | —CH₂-(4-methoxy- | N | —NH(CH₂)₂— | —NHC(═NH)NH |
| | phenyl | | phenyl) | | | (allyl) |
| 71 | 3,4-dichloro- | —CH₂— | —CH₂-(4-methoxy- | N | —NH(CH₂)₂— | —NHC(═NH)NH |
| | phenyl | | phenyl) | | | (phenyl) |
| 72 | 3,4-dichloro- | —CH₂— | —CH₂-(4-methoxy- | N | —NH(CH₂)₂— | —NHC(═NH)NH(4- |
| | phenyl | | phenyl) | | | methoxy-phenyl) |
| 73 | 3,4-dichloro- | —CH₂— | —CH₂-(4-methoxy- | N | —NH(CH₂)₂— | —NHC(═NH)NH(4- |
| | phenyl | | phenyl) | | | chloro-phenyl) |
| 74 | 3,4-dichloro- | —CH₂— | —CH₂-(4-methoxy- | N | —NH(CH₂)₂— | —NHC(═NH)NH(4- |
| | phenyl | | phenyl) | | | trifluoromethyl- |
| | | | | | | phenyl) |
| 75 | 3,4-dichloro- | —CH₂— | —CH₂-(4-methoxy- | N | —NH(CH₂)₂— | —NHC(═NH)NH |
| | phenyl | | phenyl) | | | (pyridin-3-yl) |
| 76 | 3,4-dichloro- | —CH₂— | —CH₂-(4-methoxy- | N | —NH(CH₂)₂— | —NHC(═NH)NH(4- |
| | phenyl | | phenyl) | | | methylcarbonyl- |
| | | | | | | phenyl) |
| 77 | furan-3-yl | —CH₂— | —CH₂-(4-methoxy- | N | —NH(CH₂)₂— | —NHC(═NH)NH₂ |
| | | | phenyl) | | | |
| 78 | thiophen-2-yl | —CH₂— | —CH₂-(4-methoxy- | N | —NH(CH₂)₂— | —NHC(═NH)NH₂ |
| | | | phenyl) | | | |
| 79 | 4-methoxy- | R,S-mixture | —CH₂-(4-methoxy- | N | —NH(CH₂)₂— | —NHC(═NH)NH₂ |
| | phenyl | —CH(CH₃)— | phenyl) | | | |
| 80 | 4- | —CH₂— | —CH₂-(4-methoxy- | N | —NH(CH₂)₂— | —NHC(═NH)NH₂ |
| | difluoromethoxy- | | phenyl) | | | |
| | phenyl | | | | | |
| 81 | phenyl | —CH₂— | —CH₂-(4-methoxy- | CH | —NH(CH₂)₂— | —NHC(═NH)NH₂ |
| | | | phenyl) | | | |
| 82 | 4-methoxy- | R,S-mixture | —CH₂-(4-methoxy- | N | —NH(CH₂)₂— | —NHC(═NH)NH₂ |
| | phenyl | —CH(allyl)- | phenyl) | | | |
| 83 | 4-chloro-phenyl | R,S-mixture | —CH₂-(4-methoxy- | N | —NH(CH₂)₂— | —NHC(═NH)NH₂ |
| | | —CH(allyl)- | phenyl) | | | |
| 84 | 4-methoxy- | —CH₂— | —CH₂-(4-methoxy- | CH | —NH(CH₂)₂— | —NHC(═NH)NH₂ |
| | phenyl | | phenyl) | | | |
| 85 | 4-methoxy- | —CH₂— | —CH₂-(6-methoxy- | N | —NH(CH₂)₂— | —NHC(═NH)NH₂ |
| | phenyl | | pyridin-3-yl) | | | |
| 86 | 4-methoxy- | —CH₂— | —CH₂-(4-methoxy- | N | —NH(CH₂)₂— | —NHC(═NH)NH₂ |
| | phenyl | | cyclohexyl) | | | |
| 87 | 4-fluoro-phenyl | —CH₂— | —CH₂-(4-nitro-phenyl) | N | —NH(CH₂)₂— | —NHC(═NH)NH₂ |
| 88 | 4-fluoro-phenyl | —CH₂— | —CH₂-(4-methoxy- | N | —NH(CH₂)₂— | —NHC(═NH)NH(2- |
| | | | phenyl) | | | (morpholin-4-yl)- |
| | | | | | | eth-1-yl) |
| 89 | 4-fluoro-phenyl | —CH₂— | —CH₂-(4-methoxy- | N | —NH(CH₂)₂— | —NHC(═NH)NH(3- |
| | | | phenyl) | | | (morpholin-4-yl)- |
| | | | | | | prop-1-yl) |
| 90 | 4-fluoro-phenyl | —CH₂— | —CH₂-(4-methoxy- | N | —NH(CH₂)₂— | —NHC(═NH)NH(4- |
| | | | phenyl) | | | cyano-phenyl) |
| 91 | 4-fluoro-phenyl | —CH₂— | —CH₂-(4-methoxy- | N | —NH(CH₂)₂— | —NHC(═NH)NH(4- |
| | | | phenyl) | | | nitro-phenyl) |
| 92 | 4-fluoro-phenyl | —CH₂— | —CH₂-(4-methoxy- | N | —NH(CH₂)₂— | —NHC(═NH)NH |
| | | | phenyl) | | | (1,3-benzo |
| | | | | | | dioxol-5-yl) |
| 93 | 4-fluoro-phenyl | —CH₂— | —CH₂-(4-methoxy- | N | —NH(CH₂)₂— | —NHC(═NH)NHNH₂ |
| | | | phenyl) | | | |
| 94 | 3-nitro-phenyl | —CH₂— | —CH₂-(4-methoxy- | N | —NH(CH₂)₂— | —NHC(═NH)NH₂ |
| | | | phenyl) | | | |
| 95 | 4-nitro-phenyl | —CH₂— | —CH₂-(4-methoxy- | N | —NH(CH₂)₂— | —NHC(═NH)NH₂ |
| | | | phenyl) | | | |
| 96 | 3-amino-phenyl | —CH₂— | —CH₂-(4-methoxy- | N | —NH(CH₂)₂— | —NHC(═NH)NH₂ |
| | | | phenyl) | | | |
| 97 | 4-cyano-phenyl | —CH₂— | —CH₂-(4-methoxy- | N | —NH(CH₂)₂— | —NHC(═NH)NH₂ |
| | | | phenyl) | | | |
| 98 | 3-cyano-phenyl | —CH₂— | —CH₂-(4-methoxy- | N | —NH(CH₂)₂— | —NHC(═NH)NH₂ |
| | | | phenyl) | | | |
| 99 | 4-methoxy | —CH₂— | —CH₂-(4-methoxy- | N | —NH(CH₂)₂— | —NHC(═NH)NH₂ |
| | carbonyl- | | phenyl) | | | |
| | phenyl | | | | | |
| 100 | 3-methoxy | —CH₂— | —CH₂-(4-methoxy- | N | —NH(CH₂)₂— | —NHC(═NH)NH₂ |
| | carbonyl- | | phenyl) | | | |
| | phenyl | | | | | |
| 101 | 4-carboxy- | —CH₂— | —CH₂-(4-methoxy- | N | —NH(CH₂)₂— | —NHC(═NH)NH₂ |
| | phenyl | | phenyl) | | | |
| 102 | 3,4-dichloro- | —CH₂— | —CH₂-(4-methoxy- | N | —NH(CH₂)C(Me)₂— | —NHC(═NH)NH₂ |
| | phenyl | | phenyl) | | | |
| 103 | 4-fluoro-phenyl | —CH₂— | —CH₂-(4-methoxy- | N | —NH(CH₂)₂— | —NHC(═NH)NH(4- |
| | | | phenyl) | | | bromo-phenyl) |
| 104 | 4-fluoro-phenyl | —CH₂— | —CH₂-(4-methoxy- | N | —NH(CH₂)₂— | —NHC(═NH)NH |
| | | | phenyl) | | | (pyridin-2-yl) |
| 105 | 4-fluoro-phenyl | —CH₂— | —CH₂-(4-methoxy- | N | —NH(CH₂)₂— | —NHC(═NH)NH |
| | | | phenyl) | | | (pyridin-2-yl-ethyl) |
| 106 | 4-fluoro-phenyl | —CH₂— | —CH₂-(4-methoxy- | N | —NH(CH₂)₂— | —NHC(═NH)NH(4- |
| | | | phenyl) | | | ethoxycarbonyl- |
| | | | | | | phenyl) |
| 107 | 4-fluoro-phenyl | —CH₂— | —CH₂-(4-methoxy- | N | —NH(CH₂)₂— | —NHC(═NH)NH |
| | | | phenyl) | | | (2,4-difluoro- |
| | | | | | | phenyl) |
| 108 | 4-fluoro-phenyl | —CH₂— | —CH₂-(4-methoxy- | N | —NH(CH₂)₂— | —NHC(═NH)NH(n- |
| | | | phenyl) | | | decanyl) |
| 109 | 4-t-butoxy- | —CH₂— | —CH₂-(4-methoxy- | N | —NH(CH₂)₂— | —NHC(═NH)NH₂ |
| | phenyl | | phenyl) | | | |
| 110 | 4-hydroxy- | —CH₂— | —CH₂-(4-methoxy- | N | —NH(CH₂)₂— | —NHC(═NH)NH₂ |
| | phenyl | | phenyl) | | | |
| 111 | 2-chloro- | —CH₂— | —CH₂-(4-methoxy- | N | —NH(CH₂)₂— | —NHC(═NH)NH₂ |
| | thiazol-4-yl | | phenyl) | | | |
| 112 | benzo | —CH₂— | —CH₂-(4-methoxy- | N | —NH(CH₂)₂— | —NHC(═NH)NH₂ |
| | furan-2-yl | | phenyl) | | | |
| 113 | 3,4-dichloro- | —CH₂— | —CH₂-(4-methoxy- | N | —NH(CH₂)₂— | —N(Me) |
| | phenyl | | phenyl) | | | C(═NH)NH₂ |
| 114 | 4-fluoro-phenyl | —CH₂— | —CH₂-(4-methoxy- | N | —NH(CH₂)₂— | —NHC(═NH)NH |
| | | | phenyl) | | | (CH₂CF₃) |
| 115 | 4-fluoro-phenyl | —CH₂— | —CH₂-(4-methoxy- | N | —NH(CH₂)₂— | —NHC(═NH)NH(3- |
| | | | phenyl) | | | methoxypropyl) |
| 116 | 4-fluoro-phenyl | —CH₂— | —CH₂-(4-methoxy- | N | —NH(CH₂)₂— | —NHC(═NH) |
| | | | phenyl) | | | piperidin-1-yl |
| 117 | 4-fluoro-phenyl | —CH₂— | —CH₂-(4-methoxy- | N | —NH(CH₂)₂— | —NHC(═NH)N(Me) |
| | | | phenyl) | | | phenyl |
| 118 | 4-fluoro-phenyl | —CH₂— | —CH₂-(4-methoxy- | N | —NH(CH₂)₂— | —NHC(═NH)NH(2- |
| | | | phenyl) | | | fluoro-phenyl) |
| 119 | 4-fluoro-phenyl | —CH₂— | —CH₂-(4-methoxy- | N | —NH(CH₂)₂— | —NHC(═NH)NH(4- |
| | | | phenyl) | | | fluoro-phenyl) |
| 120 | 4-fluoro-phenyl | —CH₂— | —CH₂-(4-methoxy- | N | —NH(CH₂)₂— | —NHC(═NH)NH(4- |
| | | | phenyl) | | | methyl-phenyl) |
| 121 | 4-fluoro-phenyl | —CH₂— | —CH₂-(4-methoxy- | N | —NH(CH₂)₂— | —NHC(═NH)NH(t- |
| | | | phenyl) | | | butyl) |
| 122 | 4-chloro-phenyl | —CH₂— | —CH₂-(4-amino- | N | —NH(CH₂)₂— | —NHC(═NH)NH₂ |
| | | | phenyl) | | | |
| 123 | t-butyl | —CH₂— | —CH₂-(4-methoxy- | N | —NH(CH₂)₂— | —NHC(═NH)NH₂ |
| | | | phenyl) | | | |
| 124 | cyclopentyl | —CH₂— | —CH₂-(4-methoxy- | N | —NH(CH₂)₂— | —NHC(═NH)NH₂ |
| | | | phenyl) | | | |
| 125 | 4-amino-phenyl | —CH₂— | —CH₂-(4-methoxy- | N | —NH(CH₂)₂— | —NHC(═NH)NH₂ |
| | | | phenyl) | | | |
| 126 | 4-fluoro-phenyl | —CH₂— | —CH₂-(4-methoxy- | N | —NH(CH₂)₂— | —NHC(═NH)NH |
| | | | phenyl) | | | (adamantan-2-yl) |
| 127 | 4-fluoro-phenyl | —CH₂— | —CH₂-(4-methoxy- | N | —NH(CH₂)₂— | —NHC(═NH)NH(4- |
| | | | phenyl) | | | trifluoromethoxy- |
| | | | | | | phenyl) |
| 128 | 4-fluoro-phenyl | —CH₂— | —CH₂-(4-methoxy- | N | —NH(CH₂)₂— | —NHC(═NH)NH(4- |
| | | | phenyl) | | | hydroxy-phenyl) |
| 129 | 4-chloro-phenyl | —CH₂— | —CH₂-phenyl | N | —NH(CH₂)₂— | —NHC(═NH)NH₂ |
| 130 | 4-chloro-phenyl | —CH₂— | —CH₂-furan-3-yl | N | —NH(CH₂)₂— | —NHC(═NH)NH₂ |
| 131 | 4-fluoro-phenyl | —CH₂— | —CH₂-(4-methoxy- | N | 1,4-cyclohexyl | —NHC(═NH)NH₂ |
| | | | phenyl) | | | |
| 132 | 4-fluoro-phenyl | —CH₂— | —CH₂-(4-methoxy- | N | —NHCH₂C(═O)— | —NHC(═NC(═O)O- |
| | | | phenyl) | | | t-butyl)NH₂ |
| 133 | 4-fluoro-phenyl | —CH₂— | —CH₂-(4-methoxy- | N | —NH(CH₂)₂— | —NHC(═NH)NH(2- |
| | | | phenyl) | | | methylthio-phenyl) |
| 134 | 4-fluoro-phenyl | —CH₂— | —CH₂-(4-methoxy- | N | —NH(CH₂)₂— | —NHC(═NH)NH |
| | | | phenyl) | | | (C(═O)phenyl) |
| 135 | 4-fluoro-phenyl | —CH₂— | —CH₂-(4-methoxy- | N | —NH(CH₂)₂— | —NHC(═NH)NH |
| | | | phenyl) | | | (pyrimidin-2-yl) |
| 136 | 4-fluoro-phenyl | —CH₂— | —CH₂-(4-methoxy- | N | —NH((S)—CHMe)₂— | —NHC(═NH)NH₂ |
| | | | phenyl) | | | |
| 137 | 4-fluoro-phenyl | —CH₂— | —CH₂-(4-methoxy- | N | —NH((R)—CHMe)₂— | —NHC(═NH)NH_{2′} |
| | | | phenyl) | | | |
| 138 | 4-fluoro-phenyl | —CH₂— | —CH₂-(4-methoxy- | N | —NH(CH₂)₂— | —NH(═NH)NH(4- |
| | | | phenyl) | | | trifluoromethyl- |
| | | | | | | 5,6,7,8-tetrahydro- |
| | | | | | | quinazolin-2-yl) |
| 139 | 4-fluoro-phenyl | —CH₂— | —CH₂-(4-methoxy- | N | —NH(CH₂)₂— | —NHC(═NH)NH(5- |
| | | | phenyl) | | | methyl- |
| | | | | | | pyridin-2-yl) |
| 140 | 4-fluoro-phenyl | —CH₂— | —CH₂-(4-methoxy- | N | —NH(CH₂)₂— | —NHC(═NH) |
| | | | phenyl) | | | morpholin-4-yl |
| 141 | 4-chloro-phenyl | —CH₂— | —CH₂-furan-2-yl | N | —NH(CH₂)₂— | —NHC(═NH)NH₂ |
| 142 | 4-chloro-phenyl | —CH₂— | —CH₂-(4-methoxy- | N | —NH(CH₂)₅— | —NHC(═NH)NH₂ |
| | | | phenyl) | | | |
| 143 | 4-methoxy- | —CH₂— | —CH₂-(4-hydroxy- | N | —NH(CH₂)₂— | —NHC(═NH)NH₂ |
| | phenyl | | phenyl) | | | |
| 144 | 4-chloro-phenyl | —CH₂— | —CH₂-(4-methoxy- | N | —NH(CH₂)₆— | —NHC(═NH)NH₂ |
| | | | phenyl) | | | |
| 145 | 4-methoxy- | —(CH₂)₂— | —CH₂-(4-methoxy- | N | —NH(CH₂)₂— | —NHC(═NH)NH₂ |
| | phenyl | | phenyl) | | | |
| 146 | 4-methoxy- | —(CH₂)₃— | —CH₂-(4-methoxy- | N | —NH(CH₂)₂— | —NHC(═NH)NH₂ |
| | phenyl | | phenyl) | | | |
| 147 | 3,4-dichloro- | —CH₂— | —CH₂-(4- | N | —NH(CH₂)₂— | —NHC(═NH)NH₂ |
| | phenyl | | methoxycarbonyl- | | | |
| | | | phenyl) | | | |
| 148 | phenyl | —CH₂— | —CH₂-(4-n-butyloxy- | N | —NH(CH₂)₂— | —NHC(═NH)NH₂ |
| | | | phenyl) | | | |
| 149 | 4-chloro-phenyl | —CH₂— | —CH₂-phenyl | N | —NH(CH₂)₂— | —NHC(═NH)NH₂ |
| 150 | 4-chloro-phenyl | —CH₂— | —CH₂-furan-3-yl | N | —NH(CH₂)₂— | —NHC(═NH)NH₂ |
| 151 | 4-fluoro-phenyl | —CH₂— | —CH₂-(4-methoxy- | N | —NH(CH₂)₂— | —NHC(═NH) |
| | | | phenyl) | | | NHC(═O)methyl |
| 152 | 4-fluoro-phenyl | —CH₂— | —CH₂-(4-methoxy- | N | —NH(CH₂)₂— | —NHC(═NH) |
| | | | phenyl) | | | NH(allyl) |
| 153 | 4-fluoro-phenyl | —CH₂— | —CH₂-(4-methoxy- | N | —NH(CH₂)₂— | —NHC(═NH) |
| | | | phenyl) | | | NH(i-propyl) |
| 154 | 4-fluoro-phenyl | —CH₂— | —CH₂-(4-methoxy- | N | —NH(CH₂)₂— | —NHC(═NH) |
| | | | phenyl) | | | NH(n-propyl) |
| 155 | 4-fluoro-phenyl | —CH₂— | —CH₂-(4-methoxy- | N | —NH(CH₂)₂— | —NHC(═NH) |
| | | | phenyl) | | | NH(ethyl) |
| 156 | 4-fluoro-phenyl | —CH₂— | —CH₂-(4-methoxy- | N | —NH(CH₂)₂— | —NHC(═NH) |
| | | | phenyl) | | | NH(methyl) |
| 157 | 4-methoxy- | —CH₂— | —CH₂-(4-methoxy- | CH | —C(═O)NH(CH₂)₂— | —NHC(═NH)NH₂ |
| | phenyl | | phenyl) | | | |
| 158 | 4-methoxy- | —CH₂— | —CH₂-(4-methoxy- | CH | —O(CH₂)₂— | —NHC(═NH)NH₂ |
| | phenyl | | phenyl) | | | |
| 159 | 4-methoxy- | —CH₂— | —CH₂-(4-methoxy- | CH | —S(CH₂)₂— | —NHC(═NH)NH₂ |
| | phenyl | | phenyl) | | | |
| 160 | 4-methoxy- | —CH₂— | —CH₂-(4-methoxy- | CH | —(CH₂)₃— | —NHC(═NH)NH₂ |
| | phenyl | | phenyl) | | | |

### BIOLOGICAL EXAMPLES

### Biological Example 1

### CFA-Induced Paw Radiant Heat Hypersensitivity

Each rat is placed in a test chamber on a warm glass surface and allowed to acclimate for approximately 10 min. A radiant thermal stimulus (beam of light) is then focused through the glass onto the plantar surface of each hind paw in turn. The thermal stimulus is automatically shut off by a photoelectric relay when the paw is moved or when the cut-off time is reached (20 sec for radiant heat at ˜5 amps). An initial (baseline) response latency to the thermal stimulus is recorded for each animal prior to the injection of complete Freund's adjuvant (CFA). Twenty-four hr following intraplantar CFA injection, the response latency of the animal to the thermal stimulus is then re-evaluated and compared to the animal's baseline response time. Only rats that exhibit at least a 25% reduction in response latency (i.e., were hyperalgesic) are included in further analysis. Immediately following the post-CFA latency assessment, the indicated test compound or vehicle is administered orally. Post-compound treatment withdrawal latencies are assessed at fixed time intervals, typically 30, 60, 120, 180, and 300 min.
The percent reversal (% R) of hypersensitivity is calculated using group mean values or using individual animal values, according to one of the following formulae:
1: For calculating the % R of hypersensitivity using the mean value for groups of animals at each time point:

% reversal═[(group treatment response−group CFA response)/(group baseline response−group CFA response)]×100
Results are given for the maximum % R observed at any time point tested.
2: For calculating the % R of hypersensitivity using individual animal values at each time point:

% reversal═[(individual treatment response−individual CFA response)/(individual baseline response−individual CFA response)]×100.
Results are given as a mean of the maximum % R values calculated for each individual animal±SEM.

### Biological Example 2

### CFA-Induced Paw Pressure Hypersensitivity

Prior to testing, rats are acclimated to the handling procedure twice a day for a period of two days. The test consists of placing the left hindpaw on a polytetrafluoroethylene-coated platform and applying a linearly increasing mechanical force (constant rate of 12.5 mmHg/s) in between the third and fourth metatarsal of the dorsum of the rat's hindpaw, with a dome-tipped plinth (0.7 mm in radius), using an analgesy-meter (Stoelting, Chicago, Ill.), also known as a Randall-Selitto apparatus. The endpoint is automatically reached upon hindpaw withdrawal, and the terminal force (in grams) is noted. An initial (baseline) response threshold to the mechanical stimulus is recorded for each animal prior to the injection of complete Freund's adjuvant (CFA). Forty hr following intraplantar CFA injection, the response threshold of the animal to the mechanical stimulus is re-evaluated and compared to the animal's baseline response threshold. A response is defined as a withdrawal of the hindpaw, a struggling to remove the hindpaw or vocalization. Only rats that exhibit at least a 25% reduction in response threshold (i.e., hyperalgesia) are included in further analysis. Immediately following the post-CFA threshold assessment, rats are administered the indicated test compound or vehicle. Post-treatment withdrawal thresholds are assessed at 1 hr. Paw withdrawal thresholds are converted to percent reversal of hypersensitivity according to the following formula: % reversal═[(post treatment response−predose response)/(baseline response−predose response)]×100.

### Biological Example 3

### Visceral Hyperalgesia Model

This protocol uses barostat-controlled, isobaric colorectal distensions (CRD) in rats to evaluate the potency and efficacy of test compounds in treating visceral hyperalgesia. Rats (male Sprague-Dawley (275-350 g; Charles River Labs) are housed 2 to 4 animals per cage in a temperature and humidity controlled room with a 12 hr/12 hr light/dark cycle, with ad libitum access to food and water. One day after release from quarantine, the animals are acclimated to progressively longer (30 min and 4 hr later, 45 min) periods of simple restraint in plexiglas devices (G-3, rat ECU; Braintree Scientific; Braintree Mass.). The animals are returned to their home cages overnight. The next day they are acclimated in the restraint device for 60 min in the morning. Four hr later, the animals are lightly anesthetized with 70% CO₂:30% O₂. A highly compliant, 4 cm long polyethylene balloon, lubricated with lubricating jelly, is then inserted via the anus into the rectum and distal colon. The balloon is positioned such that the aboral end is 1 cm from the anus and is secured in place by taping the balloon catheter to the base of the tail. The catheter is connected to a computerized barostat that controls the inflation of the balloon and the resulting colorectal distension. The balloon pressure, representing intracolonic pressure, is continuously recorded. CRD in conscious animals elicits a reflex visceromotor response consisting of contraction of the anterior abdominal wall muscles (Ness T J and Gebhart G F; Colorectal distension as a noxious visceral stimulus: physiologic and pharmacologic characterization of pseudaffective reflexes in the rat, Brain Res., (1988), 450: 153-169). Contraction of these muscles increases intraabdominal pressure and subsequently increases intracolonic pressure. Changes in intracolonic pressure are transduced through the same balloon used to deliver the CRD. The manometric endpoint has recently been reported to mimic electromyographic responses recorded from anterior abdominal wall muscles in rats (Tammpere A, Brusberg M, Axenborg J, Hirsch I, Larsson H and Lindstrom E, Evaluation of pseudo-affective responses to noxious colorectal distension in rats by manometric recordings, Pain, (2005), 116: 220-226). Stimulus-response data are obtained by delivering two series of 20-sec ramp (15, 30, 45, 60, 75 mmHg) distensions at four-min intervals and recording the manometric response as follows: the intracolonic pressure signal is passed through a digital 1 Hz highpass filter, rectified and the integral of the initial 15 seconds of the CRD subjected to baseline subtraction (the 15 sec immediately preceding balloon distension); the responses at each distending pressure are averaged to obtain a control stimulus/response curve for each animal. The colorectal balloons are then removed and the animals are returned to their home cages. The following morning, one treatment group is injected i.p. with test article or vehicle. One hour later, an acute colitis is induced in all treatment groups by the intracolonic instillation of a 1.5 mL bolus of 2.5% (w/v) zymosan A (from Saccharomyces cerevisiae; Sigma Chemical Co., St. Louis) in 30% ethanol (under light 70% CO₂:30% O₂ anesthesia). 4 hours later, the animals are lightly anesthetized and the colorectal balloons inserted as on the previous day for controlled distensions. The identical CRD stimuli is applied and manometric responses are recorded and analyzed as described for the control phase of the experiment. Data are excluded from experiments in which animals in the vehicle treatment group do not exhibit a hyperalgesic response following zymosan administration. Data are expressed as a percent (%±SEM) of the initial (control) manometric responses, with each animal serving as its own control.

### Biological Example 4

### Models of Nociception: Rat Formalin Test

Rats are administered vehicle or a test antinociceptive agent. Animals are then placed in observation chambers and allowed to acclimate. Formalin (50 μL of 5%) is injected beneath the skin on the top of one hindpaw. The resulting biphasic pattern of activity, consisting of lifting, licking, biting and/or guarding (Wheeler-Aceto and Cowan, 1991) is quantified with an Automated Flinch Detecting System for 60 minutes. (Yaksh et al., 2001). Responses may be grouped by time into Phase I (1-9 min.), Phase II (10-60 min.) and/or Phase IIA (10-40 min.). Data are calculated as the percent maximum possible effect:

% MPE═100×(Mean Animal Drug Treated Count)/(Mean Animal vehicle Treated Count)

### REFERENCES

Wheeler-Aceto H and Cowan A. Standardization of the rat paw formalin test for the evaluation of analgesics. Psychopharmacol. 1991; 104:35-44.

Yaksh T L, Ozaki G, McCumber D, Rathbun M, Svensson C, Malkmus S, and Yaksh M C. An automated flinch detecting system for use in the formalin nociceptive bioassay. J Applied Physiology. 2001; 90:2386-402.

### Biological Example 5

### Antinociceptive Tests: Mouse Acetylcholine-Induced Abdominal Irritant Test

The procedure used is that described by Collier et al. (1968), with minor modifications. Thirty minutes after the administration of test drug, the animals receive an i.p. injection of 5.5 mg/kg of acetylcholine bromide. The mice are then placed into large glass animal jars and continuously observed for the first occurrence of a characteristic behavioral response (i.e., twisting and elongation of the body, which extends throughout the hindlimbs) within the specified observation period of 10 minutes. The percent of inhibition of this response is calculated as follows:

% Inhibition═100×(Number of Nonresponders)/Number of Animals in Group)

The estimated ED₅₀ value (the dose of agonist calculated to produce 50% antinociception) and the corresponding 95% fiducial intervals are determined using the probit analysis of Litchfield and Wilcoxon (1949).

### REFERENCES

Litchfield J T and Wilcoxon F. A simplified method of evaluating dose-effect experiments. J Pharmacol Exp Ther 95: 1098-1104, 1949.

### Biological Example 6

### Antinociceptive Tests: Mouse 48° C. Hot-Plate Test

The procedure used is that described by Eddy and Leimbach (1953) and O'Callaghan and Holtzman (1975), with minor modifications. Mice are placed on a heated surface (48° C.), and the time interval (seconds) between placement and the prototypic behavior (e.g., a shaking, licking or tucking of the hind paw) is recorded as the predrug latency response. This same procedure is repeated at 30 minutes after test drug is administered p.o., 10 mL/kg. The percent maximum possible antinociceptive effect (% MPE) is determined using the formula:

% MPE═100×(Test latency−Predrug latency)/(Cutoff time−Predrug Latency)

using the predrug latency of each animal and cut-off time established to prevent injury to the animal (i.e., 90 seconds). The ED₅₀ value and 95% confidence intervals are determined using a computer-assisted linear regression analysis of the dose-response curve, including an analysis of variance test for linearity.

### REFERENCES

Collier H O, Dinneen L C, Johnson C A and Schneider, C. The abdominal irritant response and its suppression by analgesic drugs in the mouse. Br J Pharmacol 32:295-310, 1968.

Eddy N B, Leimbach D. Synthetic analgesics II. Dithienylbutenyl- and dithienylbutylamines J Pharmacol Exp Ther 1953; 107:385-393.

O'Callaghan J P, Holtzman S G. Quantification of the analgesic activity of narcotic antagonists by a modified hot-plate procedure. J Pharmacol Exp Ther 1975; 192:497-505.

TABLE 2
Mass Spectral Data

| Cpd | MS obs | MS calc |
|---|---|---|
| 1 | 411.9 | 412.19 |
| 2 | 424.3 | 424.21 |
| 3 | 452.0 | 452.20 |
| 4 | 438.0 | 438.23 |
| 5 | 390.1 | 390.23 |
| 6 | 414.0 | 414.19 |
| 7 | 462.0 | 462.19 |
| 8 | 450.1 | 450.26 |
| 9 | 438.9 | 439.18 |
| 10 | 440.2 | 440.20 |
| 11 | 395.2 | 395.19 |
| 12 | 438.3 | 438.23 |
| 13 | 460.2 | 460.19 |
| 14 | 465.9 | 466.26 |
| 15 | 461.9 | 462.19 |
| 16 | 442.0 | 442.20 |
| 17 | 442.0 | 442.20 |
| 18 | 492.0 | 492.13 |
| 19 | 477.8 | 478.18 |
| 20 | 454.0 | 454.22 |
| 21 | 454.0 | 454.22 |
| 22 | 436.9 | 437.21 |
| 23 | 450.9 | 451.22 |
| 24 | 456.0 | 456.22 |
| 25 | 437.9 | 438.23 |
| 26 | 437.9 | 438.23 |
| 27 | 508.2 | 508.13 |
| 28 | 517.1 | 517.13 |
| 29 | 505.8 | 506.15 |
| 30 | 457.8 | 458.17 |
| 31 | 453.9 | 454.22 |
| 32 | 519.7 | 520.16 |
| 33 | 455.9 | 456.22 |
| 34 | 519.7 | 520.16 |
| 35 | 519.7 | 520.16 |
| 36 | 545.8 | 546.18 |
| 37 | 507.7 | 508.11 |
| 38 | 489.7 | 490.15 |
| 39 | 457.8 | 458.17 |
| 40 | 545.7 | 546.10 |
| 41 | 527.7 | 528.11 |
| 42 | 545.8 | 546.18 |
| 43 | 547.8 | 546.18 |
| 44 | 506.1 | 506.15 |
| 45 | 469.8 | 470.20 |
| 46 | 452.0 | 452.24 |
| 47 | 547.7 | 546.18 |
| 48 | 532.1 | 532.13 |
| 49 | 518.1 | 518.15 |
| 50 | 518.7 | 519.14 |
| 51 | 504.8 | 505.13 |
| 52 | 505.8 | 506.15 |
| 53 | 520.1 | 520.16 |
| 54 | 534.1 | 534.18 |
| 55 | 517.7 | 518.15 |
| 56 | 511.8 | 512.22 |
| 57 | 527.7 | 528.11 |
| 58 | 534.2 | 534.18 |
| 59 | 519.7 | 520.16 |
| 60 | 517.7 | 518.15 |
| 61 | 548.2 | 548.19 |
| 62 | 574.2 | 574.21 |
| 63 | 582.1 | 582.18 |
| 64 | 576.1 | 576.19 |
| 65 | 596.1 | 596.19 |
| 66 | 572.1 | 572.16 |
| 67 | 550.1 | 550.17 |
| 68 | 505.8 | 506.15 |
| 69 | 455.9 | 456.17 |
| 70 | 532.2 | 532.16 |
| 71 | 568.2 | 568.16 |
| 72 | 598.1 | 598.17 |
| 73 | 602.1 | 602.12 |
| 74 | 636.1 | 636.15 |
| 75 | 569.2 | 569.16 |
| 76 | 610.1 | 610.17 |
| 77 | 413.9 | 414.19 |
| 78 | 429.8 | 430.17 |
| 79 | 467.9 | 468.24 |
| 80 | 489.7 | 490.20 |
| 81 | 422.9 | 423.21 |
| 82 | 493.8 | 494.25 |
| 83 | 497.7 | 498.20 |
| 84 | 452.9 | 453.23 |
| 85 | 455.2 | 455.22 |
| 86 | 459.9 | 460.27 |
| 87 | 456.9 | 457.17 |
| 88 | 555.2 | 555.28 |
| 89 | 569.3 | 569.30 |
| 90 | 543.2 | 543.23 |
| 91 | 563.2 | 563.22 |
| 92 | 562.2 | 562.22 |
| 93 | 457.2 | 457.21 |
| 94 | 468.7 | 469.19 |
| 95 | 468.7 | 469.19 |
| 96 | 438.9 | 439.22 |
| 97 | 448.8 | 449.21 |
| 98 | 448.8 | 449.21 |
| 99 | 481.8 | 482.22 |
| 100 | 481.8 | 482.22 |
| 101 | 468.9 | 468.20 |
| 102 | 519.7 | 520.16 |
| 103 | 596.1 | 596.14 |
| 104 | 519.2 | 519.23 |
| 105 | 547.2 | 547.26 |
| 106 | 590.3 | 590.25 |
| 107 | 554.2 | 554.21 |
| 108 | 582.3 | 582.36 |
| 109 | 495.9 | 496.27 |
| 110 | 440.9 | 440.20 |
| 111 | 464.7 | 465.12 |
| 112 | 463.8 | 464.20 |
| 113 | 505.8 | 506.15 |
| 114 | 524.2 | 524.20 |
| 115 | 514.2 | 514.26 |
| 116 | 510.2 | 510.26 |
| 117 | 532.2 | 532.25 |
| 118 | 536.2 | 536.22 |
| 119 | 536.2 | 536.22 |
| 120 | 532.2 | 532.25 |
| 121 | 498.2 | 498.26 |
| 122 | 443.1 | 443.17 |
| 123 | 404.0 | 404.24 |
| 124 | 416.0 | 416.24 |
| 125 | 438.9 | 439.22 |
| 126 | 576.3 | 576.31 |
| 127 | 602.1 | 602.21 |
| 128 | 534.2 | 534.23 |
| 129 | 427.8 | 428.16 |
| 130 | 417.9 | 418.14 |
| 131 | 496.3 | 495.9 |
| 132 | 556.2 | 556.2 |
| 133 | 564.2 | 564.22 |
| 134 | 546.2 | 546.23 |
| 135 | 520.2 | 520.22 |
| 136 | 470.2 | 470.23 |
| 137 | 470.2 | 470.23 |
| 138 | 642.2 | 642.26 |
| 139 | 533.2 | 533.24 |
| 140 | 512.2 | 512.24 |
| 141 | 417.9 | 417.85 |
| 142 | 500.1 | 500.22 |
| 143 | 440.1 | 440.20 |
| 144 | 514.2 | 514.23 |
| 145 | 467.9 | 468.24 |
| 146 | 482.0 | 482.25 |
| 147 | 520.3 | 520.10 |
| 148 | 465.9 | 465.56 |
| 149 | 427.8 | 427.89 |
| 150 | 417.9 | 417.85 |
| 151 | 484.2 | 484.21 |
| 152 | 482.2 | 482.23 |
| 153 | 484.2 | 484.24 |
| 154 | 484.2 | 484.24 |
| 155 | 470.2 | 470.23 |
| 156 | 456.2 | 456.21 |
| 157 | 481.2 | 480.21 |
| 158 | 453.9 | 453.49 |
| 159 | 469.8 | 469.56 |
| 160 | 452.0 | 451.22 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating, ameliorating, or preventing pain, comprising administering to a subject in need thereof, a therapeutically effective amount of a compound of Formula (I) 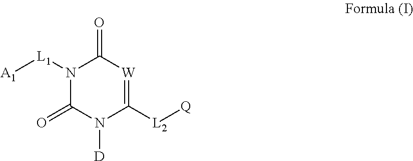
or an enantiomer, diastereomer, or pharmaceutically acceptable salt thereof;
wherein:
A₁ is hydrogen; aryl; heteroaryl; C₅₋₈cycloalkyl; or heterocyclyl; provided that A₁ is other than piperidin-4-yl, N-t-butoxycarbonyl-piperidin-4-yl, or N-methyl-piperidin-3-yl; and wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl;
L₁ is —(CH₂)ᵣ— or —CH₂CH₂X(CH₂)ₛ—, optionally substituted with one to three substituents independently selected from the group consisting of C₁₋₆alkyl, C₂₋₆alkenyl, C₂₋₆alkynyl, and halogen; provided that when A₁ is hydrogen, r is greater than or equal to 4;
r is an integer of 1 to 5;
s is an integer of 1 to 3;
X is O or S;
D is —P-A₂; wherein when A₂ is hydrogen, P is —(CH₂)₄₋₆—, and when A₂ is other than hydrogen, P is —(CH₂)₁₋₂— or —CH₂CH═CH—;
A₂ is hydrogen; benzodioxalyl; heteroaryl other than unsubstituted pyridin-2-yl; C₃₋₈cycloalkyl; or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; wherein benzodioxalyl, heteroaryl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy;
provided that no more than two substituents on A₂ are aryl(C₁₋₆)alkoxy, phenyl, or a non fused C₃₋₆cycloalkyloxy;
provided that when A₁ is unsubstituted phenyl and L₂ is —X₁—CH(R^{x})—(CR^{y}R^{z})— wherein X₁ is NH, and R^{x}, R^{y}, and R^{z} are each hydrogen, A₂ is other than unsubstituted phenyl; phenyl substituted with aryl(C₁₋₆)alkoxy or phenyl; or phenyl substituted at the meta position with cyano;
and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
and, provided that when A₁ is 3,4-dichloro-phenyl and P is —CH₂—, A₂ is other than phenyl substituted at the meta position with trifluoromethyl or trifluoromethoxy;
and, further provided that when A₁ is 3,4-dichloro-phenyl and P is —(CH₂)₂—, A₂ is other than 4-methoxy-phenyl;
W is N or C(R_{W}); wherein R_{W} is H or C₁₋₂alkyl;
L₂ is a bivalent radical selected from the group consisting of
-pyrrolidinyl or piperidinyl attached to the triazine ring of Formula (I) via its nitrogen atom, wherein said pyrrolidinyl or piperidinyl is substituted on a carbon atom with —(CH₂)₀₋₂—;
—NH—C₅₋₇cycloalkyl-(CH₂)₀₋₂—; such that when C₅₋₇cycloalkyl is cyclohexyl, Q is attached at either the 2- or cis-4-position relative to the position of —NH—;
—X₁—(CH₂)ᵤ—X₂—(CH₂)ᵥ—; wherein u is an integer of 1 to 3; and wherein v is an integer of 1 to 4; provided that when X₁ is a direct bond and W is C(R_{w}), then u is 1 and v is 2 to 4;
—X₂—(CH₂)₀₋₄—;
—X₁—(CH₂)₂₋₃—X₃—(CH₂)₂₋₃—;
—NH(CH₂)₁₋₄C(═O)—, provided that at least one of R^{b}, R^{c}, or R^{d} is other than hydrogen and m is 0;
—NHC(═O)—(CH₂)₁₋₄—;
—C(═O)NH(CR^{y}R^{z})₂₋₅—;
and
—X₁—CH(R^{x})—(CR^{y}R^{z})₁₋₅—; such that when X₁ is a direct bond and W is C(R_{w}), then R^{x} is hydrogen;
wherein X₁ is —NH—, O, S, or a direct bond, such that X₁ is other than O when W is N;
X₂ is —CH═CH—;
X₃ is O, S, NH, or C═O;
R^{x}, R^{y}, and R^{z} are independently H or C₁₋₄alkyl;
and provided that L₂ in any instance does not exceed 7 atoms in length;
and further provided that when L₂ is —X₂—(CH₂)₀₋₄— or —C(═O)NH(CR^{y}R^{z})₂₋₅—; then R_{W} is hydrogen;
Q is —(O)ₘN(R^{a})-G; and m is 0 or 1;
G is —C(═NR^{b})NR^{c}R^{d};
R^{a} and R^{d} are independently hydrogen, C₁₋₆alkyl, C₂₋₆alkenyl, or C₃₋₆alkynyl, wherein substituents of R^{a} and R^{d} other than hydrogen are optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
R^{b} is hydrogen, C₁₋₆alkyl, C₂₋₆alkenyl, C₃₋₆alkynyl, C₂₋₆alkoxycarbonyl, or cyano; or, R^{b} and R^{c} are taken together with the atoms to which they are attached to form a 5-8 membered monocyclic ring optionally substituted with oxo;
R^{c} is hydrogen, C₁₋₁₀alkyl, C₂₋₁₀alkenyl, C₃₋₁₀alkynyl, C₃₋₇cycloalkyl, adamantyl, amino, C₁₋₆alkylamino, di(C₁₋₆alkyl)amino, C₁₋₆alkylcarbonyl, C₁₋₆alkoxycarbonyl, arylcarbonyl, heteroarylcarbonyl, heterocyclylcarbonyl, aryl, heteroaryl, or heterocyclyl; wherein C₁₋₁₀alkyl, C₂₋₁₀alkenyl, and C₂₋₁₀alkynyl are optionally substituted with one to three substituents independently selected from the group consisting of hydroxy, C₁₋₆alkoxy, trifluoromethyl, aryl, heteroaryl, and heterocyclyl; and wherein any aryl- or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, aminocarbonyl, C₁₋₆alkylaminocarbonyl, di(C₁₋₆alkyl)aminocarbonyl, C₁₋₆alkoxycarbonylamino, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl, nitro, methylthio, hydroxy, and cyano; or, R^{c} and R^{d} are taken together with the atoms to which they are attached to form a 5-8 membered monocyclic ring that optionally includes 1 to 2 O or S heteroatoms within the ring, and said ring is optionally substituted with oxo;
with the proviso that in any instance, only one ring optionally exists between R^{a} and R^{b}, R^{b} and R^{c}, or R^{c} and R^{d}.

2. A method as in claim 1 wherein the pain is inflammatory pain.

3. A method as in claim 1 wherein the pain is visceral pain.

4. A method as in claim 1 wherein the pain is acute pain.

5. The use of a compound as in claim 1 for the preparation of a medicament or pharmaceutical composition for the treatment of inflammatory pain, visceral pain, or acute pain, in a subject in need thereof.

6. A method of treating or preventing pain comprising administering to a subject in need thereof, a therapeutically effective amount of a compound of Formula (Ia) 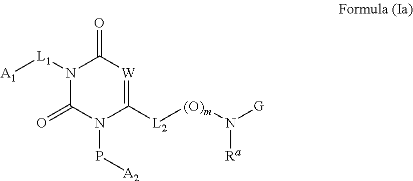
or an enantiomer, diastereomer, or pharmaceutically acceptable salt thereof;
wherein:
A₁ is phenyl or benzofuranyl; wherein phenyl is substituted at either the 4-position or 3- and 4-positions with one to two substituents independently selected from the group consisting of ethyl, methoxy, fluoro, chloro, nitro, difluoromethoxy, and methylthio;
L₁ is —CH₂— optionally substituted with methyl or allyl; P is —CH₂—;
A₂ is phenyl substituted at the para position with a substituent selected from the group consisting of methoxy, ethoxy, isopropyloxy, difluoromethoxy, hydroxy, and aminocarbonyl; or A₂ is pyridin-3-yl or pyridin-4-yl substituted with methoxy;
W is N or CH;
L₂ is a bivalent radical selected from the group consisting of
—NH-cyclohexyl-(CH₂)₀₋₂— and Q is attached at either the 2- or cis-4-position relative to the position of —NH—;
—X₁—CH(R^{x})—(CR^{y}R^{z})—; wherein X₁ is —NH— or S; and R^{x}, R^{y}, and R^{z} are each hydrogen;
and
—X₁—(R,R—CH(R^{x})CR^{y}(R^{z}))—; wherein X₁ is —NH—, and R^{x} and R^{z} are methyl, and R^{y} is hydrogen;
m is 0;
G is —C(═NR^{b})NR^{c}R^{d};
R^{a} and R^{d} are independently hydrogen, methyl or ethyl;
R^{b} is hydrogen;
R^{c} is hydrogen, C₁₋₄alkyl, C₂₋₄alkenyl, cyclohexyl, phenylcarbonyl, phenyl, pyrimidinyl, furanyl, benzo[1,3]dioxolyl, or pyridinyl; wherein C₁₋₄alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring.

7. A method as in claim 6 wherein the pain is inflammatory pain.

8. A method as in claim 6 wherein the pain is visceral pain.

9. A method as in claim 6 wherein the pain is acute pain.

10. The use of a compound as in claim 6 for the preparation of a medicament or pharmaceutical composition for the treatment of inflammatory pain, visceral pain, or acute pain, in a subject in need thereof.

11. A method of treating or preventing pain comprising administering to a subject in need thereof, a therapeutically effective amount of a compound of Formula (I) 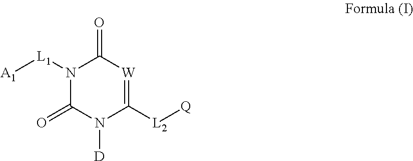
selected from the group consisting of:
a compound of Formula (I) wherein A₁ is phenyl, L₁ is —CH₂—, D is —CH₂-(4-fluoro-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methylcarboxy-phenyl), W is N, L₂ is —NH(CH₂)₂—, and Q is —NHC(═NH)NH₂;
a compound of Formula (I) wherein A₁ is phenyl, L₁ is —(CH₂)₂—, D is —CH₂-(4-methoxy-phenyl), W is N, L₂ is —NH(CH₂)₂—, and Q is —NHC(═NH)NH₂;
a compound of Formula (I) wherein A₁ is H, L₁ is —(CH₂)₄—, D is —CH₂-(4-methoxy-phenyl), W is N, L₂ is —NH(CH₂)₂—, and Q is —NHC(═NH)NH₂;
a compound of Formula (I) wherein A₁ is furan-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(3-trifluoromethyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-t-butyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-nitro-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is N, L₂ is —NH(CH₂)₂—, and Q is —ONHC(═NH)NH₂;
a compound of Formula (I) wherein A₁ is phenyl, L₁ is —CH₂—, D is —CH₂-pyridin-4-yl, W is N, L₂ is —NH(CH₂)₂—, and Q is —NHC(═NH)NH₂;
a compound of Formula (I) wherein A₁ is phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-difluoromethoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-n-butyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-trifluoromethyl-phenyl), W is N, L₂ is —NH(CH₂)₂—, and Q is —NHC(═NH)NH₂;
a compound of Formula (I) wherein A₁ is 2-fluoro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-trifluoromethoxy-phenyl), W is N, L₂ is —NH(CH₂)₂—, and Q is —NHC(═NH)NH₂;
a compound of Formula (I) wherein A₁ is 3-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 2-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-aminocarbonyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methylcarboxylamino-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —ONHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═N—CN)NH₂;
a compound of Formula (I) wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₄—, and Q is —NHC(═NH)NH₂;
a compound of Formula (I) wherein A₁ is 4-fluoro-phenyl, L₁ is —CH₂—, D is —(CH₂)₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-n-propyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-i-propyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-cyclopentyloxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methylthio-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-ethyl-phenyl), W is N, L₂ is —NH(CH₂)₂—, and Q is —NHC(═NH)NH₂;
a compound of Formula (I) wherein A₁ is 3-chloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-trifluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-difluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is cis-racemic-1,2-cyclohexyl, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is trans(1S,2S)-cyclohexyl-, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methylthio-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-ethyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is trans(1R,2R)-cyclohexyl-, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NH(3,5-dihydro-imidazol-4-on-2-yl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NH(4,5-dihydro-1H-imidazol-2-yl);
a compound of Formula (I) wherein A₁ is 3,4-dichloro-phenyl, L₁ is —CH₂—, D is —CH₂-(4-methylcarbonylamino-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-aminocarbonyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(3-ethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH-ethyl;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH-propyl;
a compound of Formula (I) wherein A₁ is 3,4-dichloro-phenyl, L₁ is —CH₂—, D is —CH₂-(4-methoxy-phenyl), W is N, L₂ is pyrrolindin-1-yl, and Q is 3-NHC(═NH)NH₂;
a compound of Formula (I) wherein A₁ is 4-chloro-phenyl, L₁ is —CH₂—, D is —CH₂-(4-methoxy-phenyl), W is N, L₂ is trans(1R,2R)-cyclohexyl-, and Q is —NHC(═NH)NH₂;
a compound of Formula (I) wherein A₁ is 3,4-dichloro-phenyl, L₁ is —CH₂—, D is —CH₂-(3-difluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(i-propyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —N(ethyl)C(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is 2-imino-imidazolid-1-yl;
a compound of Formula (I) wherein a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(n-butyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(cyclohexyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(benzyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(tetrahydrofuran-2-ylmethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenylethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(furan-2-ylmethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(2-methoxy-ethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₃—, and Q is —NHC(═NH)NH₂;
a compound of Formula (I) wherein A₁ is 3,4-dichloro-phenyl, L₁ is —CH₂—, D is —(CH₂)₆-H, W is N, L₂ is —NH(CH₂)₃—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(allyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methoxy-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-chloro-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-trifluoromethyl-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(pyridin-3-yl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methylcarbonyl-phenyl);
a compound of Formula (I) wherein A₁ is furan-3-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is thiophen-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is R,S-mixture —CH(CH₃)—, D is —CH₂-(4-methoxy-phenyl), W is N, L₂ is —NH(CH₂)₂—, and Q is —NHC(═NH)NH₂;
a compound of Formula (I) wherein A₁ is 4-difluoromethoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is CH, L₂ is —NH(CH₂)₂—, and Q is —NHC(═NH)NH₂;
a compound of Formula (I) wherein A₁ is 4-methoxy-phenyl, L₁ is R,S-mixture —CH(allyl)-, D is —CH₂-(4-methoxy-phenyl), W is N, L₂ is —NH(CH₂)₂—, and Q is —NHC(═NH)NH₂;
a compound of Formula (I) wherein A₁ is 4-chloro-phenyl, L₁ is R,S-mixture —CH(allyl)-, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(6-methoxy-pyridin-3-yl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-cyclohexyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-nitro-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-(morpholin-4-yl)-eth-1-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—; and Q is —NHC(═NH)NH(3-(morpholin-4-yl)-prop-1-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-cyano-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-nitro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(1,3-benzodioxol-5-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHNH₂;
a compound of Formula (I) wherein A₁ is 3-nitro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-nitro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3-amino-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-cyano-phenyl, L₁ is —CH₂—, D is —CH₂-(4-methoxy-phenyl), W is N, L₂ is v-NH(CH₂)₂—, and Q is —NHC(═NH)NH₂;
a compound of Formula (I) wherein A₁ is 3-cyano-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-carboxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)C(Me)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-bromo-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl-ethyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-ethoxycarbonyl-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2,4-difluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-decanyl);
a compound of Formula (I) wherein A₁ is 4-t-butoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-hydroxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 2-chloro-thiazol-4-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is benzofuran-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —N(Me)C(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(CH₂CF₃);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(3-methoxypropyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)piperidin-1-yl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)N(Me)phenyl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-fluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-fluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-methyl-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(t-butyl);
a compound of Formula (I) wherein A₁ is 4-chloro-phenyl, L₁ is —CH₂—, D is —CH₂-(4-amino-phenyl), W is N, L₂ is —NH(CH₂)₂—, and Q is —NHC(═NH)NH₂;
a compound of Formula (I) wherein A₁ is t-butyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is cyclopentyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is A₁ is 4-amino-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(adamantan-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-trifluoromethoxy-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-hydroxy-phenyl);
a compound of Formula (I) wherein A₁ is 4-chloro-phenyl, L₁ is —CH₂—, D is —CH₂-phenyl, W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is 1,4-cyclohexyl, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NHCH₂C(═O)—, and Q is —NHC(═NC(═O)O-t-butyl)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-methylthio-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(C(═O)phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyrimidin-2-yl);
a compound of Formula (I) wherein A₁ is 4-fluoro-phenyl, L₁ is —CH₂—, D is —CH₂-(4-methoxy-phenyl), W is N, L₂ is —NH((S)—CHMe)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH((R)—CHMe)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NH(═NH)NH(4-trifluoromethyl-5,6,7,8-tetrahydro-quinazolin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(5-methyl-pyridin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)morpholin-4-yl;
a compound of Formula (I) wherein A₁ is 4-chloro-phenyl, L₁ is —CH₂—, D is —CH₂-furan-2-yl, W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₅—, and Q is —NHC(═NH)NH₂;
a compound of Formula (I) wherein A₁ is 4-methoxy-phenyl, L₁ is —CH₂—, D is —CH₂-(4-hydroxy-phenyl), W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₆—, and Q is —NHC(═NH)NH₂;
a compound of Formula (I) wherein A₁ is 4-methoxy-phenyl, L₁ is —(CH₂)₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —(CH₂)₃—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxycarbonyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-n-butyloxy-phenyl), W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-phenyl, W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHC(═O)methyl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(allyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(i-propyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-propyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(ethyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(methyl);
a compound of Formula (I) wherein A₁ is 4-methoxy-phenyl, L₁ is —CH₂—, D is —CH₂-(4-methoxy-phenyl), W is CH, L₂ is -C(═O)NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is -O(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is -S(CH₂)₂—, and Q is —NHC(═NH)NH₂; and
a compound of Formula (I) wherein A₁ is 4-methoxy-phenyl, L₁ is —CH₂—, D is —CH₂-(4-methoxy-phenyl), W is CH, L₂ is ⁻(CH₂)₃⁻, and Q is —NHC(═NH)NH₂.

12. A method as in claim 11 wherein the pain is inflammatory pain.

13. A method as in claim 11 wherein the pain is visceral pain.

14. A method as in claim 11 wherein the pain is acute pain.

15. The use of a compound as in claim 11 for the preparation of a medicament or pharmaceutical composition for the treatment of inflammatory pain, visceral pain, or acute pain, in a subject in need thereof.

16. A method of treating, ameliorating, or preventing pain, comprising administering to a subject in need thereof, a pharmaceutical composition comprising a therapeutically effective amount of a compound of Formula (I) 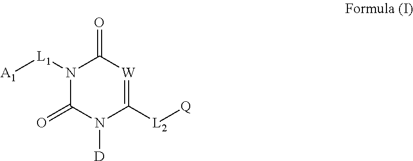
or an enantiomer, diastereomer, or pharmaceutically acceptable salt thereof;
wherein:
A₁ is hydrogen; aryl; heteroaryl; C₅₋₈cycloalkyl; or heterocyclyl; provided that A₁ is other than piperidin-4-yl, N-t-butoxycarbonyl-piperidin-4-yl, or N-methyl-piperidin-3-yl; and wherein substituents of A₁ other than hydrogen are optionally substituted with one to three substituents independently selected from the group consisting of C₁₋₆alkyl, hydroxy(C₁₋₆)alkyl, C₁₋₆alkoxy, halogen, nitro, halogenated C₁₋₆alkyl, halogenated C₁₋₆alkoxy, C₁₋₆alkylthio, C₁₋₆alkoxycarbonyl, amino, C₁₋₆alkylamino, di(C₁₋₆alkyl)amino, cyano, hydroxy, aminocarbonyl, C₁₋₆alkylaminocarbonyl, di(C₁₋₆alkyl)aminocarbonyl, C₁₋₆alkoxycarbonylamino, C₁₋₆alkylcarbonyl, C₁₋₆alkylthiocarbonyl, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl;
L₁ is —(CH₂)ᵣ— or —CH₂CH₂X(CH₂)ₛ—, optionally substituted with one to three substituents independently selected from the group consisting of C₁₋₆alkyl, C₂₋₆alkenyl, C₂₋₆alkynyl, and halogen; provided that when A₁ is hydrogen, r is greater than or equal to 4;
r is an integer of 1 to 5;
s is an integer of 1 to 3;
X is O or S;
D is —P-A₂; wherein when A₂ is hydrogen, P is —(CH₂)₄₋₆—, and when A₂ is other than hydrogen, P is —(CH₂)₁₋₂— or —CH₂CH═CH—;
A₂ is hydrogen; benzodioxalyl; heteroaryl other than unsubstituted pyridin-2-yl; C₃₋₈cycloalkyl; or phenyl optionally substituted at the meta and para positions with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy; wherein benzodioxalyl, heteroaryl, and C₃₋₈cycloalkyl are optionally substituted with one to three substituents independently selected from the group consisting of C₁₋₆alkyl, C₁₋₆alkoxy, halogen, halogenated C₁₋₆alkyl, halogenated C₁₋₆alkoxy, aryl(C₁₋₆)alkoxy, phenyl, C₁₋₆alkylthio, C₁₋₆alkoxycarbonyl, amino, C₁₋₆alkylamino, di(C₁₋₆alkyl)amino, cyano, hydroxy, nitro, C₁₋₆alkylcarbonyl, C₁₋₆alkylthiocarbonyl, aminocarbonyl, C₁₋₆alkylaminocarbonyl, di(C₁₋₆alkyl)aminocarbonyl, C₁₋₆alkylcarbonylamino, and a non fused C₃₋₆cycloalkyloxy;
provided that no more than two substituents on A₂ are aryl(C₁₋₆)alkoxy, phenyl, or a non fused C₃₋₆cycloalkyloxy;
provided that when A₁ is unsubstituted phenyl and L₂ is —X₁—CH(R^{x})—(CR^{y}R^{z})— wherein X₁ is NH, and R^{x}, R^{y}, and R^{z} are each hydrogen, A₂ is other than unsubstituted phenyl; phenyl substituted with aryl(C₁₋₆)alkoxy or phenyl; or phenyl substituted at the meta position with cyano;
and, further provided that when A₁ is unsubstituted phenyl and L₂ is —X₁—CH(R^{x})—(CR^{y}R^{z})₂— wherein X₁ is NH and R^{x}, R^{y}, and R^{z} are each hydrogen, A₂ is other than phenyl substituted with methoxy;
and, provided that when A₁ is 3,4-dichloro-phenyl and P is —CH₂—, A₂ is other than phenyl substituted at the meta position with trifluoromethyl or trifluoromethoxy;
and, further provided that when A₁ is 3,4-dichloro-phenyl and P is —(CH₂)₂—, A₂ is other than 4-methoxy-phenyl;
W is N or C(R_{W}); wherein R_{W} is H or C₁₋₂alkyl;
L₂ is a bivalent radical selected from the group consisting of
-pyrrolidinyl or piperidinyl attached to the triazine ring of Formula (I) via its nitrogen atom, wherein said pyrrolidinyl or piperidinyl is substituted on a carbon atom with —(CH₂)₀₋₂—;
—NH—C₅₋₇cycloalkyl-(CH₂)₀₋₂—; such that when C₅₋₇cycloalkyl is cyclohexyl, Q is attached at either the 2- or cis-4-position relative to the position of —NH—;
—X₁—(CH₂)ᵤ—X₂—(CH₂)ᵥ—; wherein u is an integer of 1 to 3; and wherein v is an integer of 1 to 4; provided that when X₁ is a direct bond and W is C(R_{w}), then u is 1 and v is 2 to 4;
—X₂—(CH₂)₀₋₄—;
—X₁—(CH₂)₂₋₃—X₃—(CH₂)₂₋₃—;
—NH(CH₂)₁₋₄C(═O)—, provided that at least one of R^{b}, R^{c}, or R^{d} is other than hydrogen and m is 0;
—NHC(═O)—(CH₂)₁₋₄—;
—C(═O)NH(CR^{y}R^{z})₂₋₅—;
and
—X₁—CH(R^{x})—(CR^{y}R^{z})₁₋₅—; such that when X₁ is a direct bond and W is C(R_{w}), then R^{x} is hydrogen;
wherein X₁ is —NH—, O, S, or a direct bond, such that X₁ is other than O when W is N;
X₂ is —CH═CH—;
X₃ is O, S, NH, or C═O;
R^{x}, R^{y}, and R^{z} are independently H or C₁₋₄alkyl;
and provided that L₂ in any instance does not exceed 7 atoms in length;
and further provided that when L₂ is —X₂—(CH₂)₀₋₄— or —C(═O)NH(CR^{y}R^{z})₂₋₅—, then R_{W} is hydrogen;
Q is —(O)ₘ(R^{a})-G; and m is 0 or 1;
G is —C(═NR^{b})NR^{c}R^{d};
R^{a} and R^{d} are independently hydrogen, C₁₋₆alkyl, C₂₋₆alkenyl, or C₃₋₆alkynyl, wherein substituents of R^{a} and R^{d} other than hydrogen are optionally substituted with one to three substituents independently selected from the group consisting of hydroxy, C₁₋₄alkoxy, fluoro, amino, C₁₋₄alkylamino, diC₁₋₄alkylamino, and C₁₋₄alkylcarbonyl; or R^{a} and R^{c} are taken together with the atoms to which they are attached to form a 5-8 membered monocyclic ring optionally substituted with oxo;
R^{b} is hydrogen, C₁₋₆alkyl, C₂₋₆alkenyl, C₃₋₆alkynyl, C₂₋₆alkoxycarbonyl, or cyano; or, R^{b} and R^{c} are taken together with the atoms to which they are attached to form a 5-8 membered monocyclic ring optionally substituted with oxo;
R^{c} is hydrogen, C₁₋₁₀alkyl, C₂₋₁₀alkenyl, C₃₋₁₀alkynyl, C₃₋₇cycloalkyl, adamantyl, amino, C₁₋₆alkylamino, di(C₁₋₆alkyl)amino, C₁₋₆alkylcarbonyl, C₁₋₆alkoxycarbonyl, arylcarbonyl, heteroarylcarbonyl, heterocyclylcarbonyl, aryl, heteroaryl, or heterocyclyl; wherein C₁₋₁₀alkyl, C₂₋₁₀alkenyl, and C₂₋₁₀alkynyl are optionally substituted with one to three substituents independently selected from the group consisting of hydroxy, C₁₋₆alkoxy, trifluoromethyl, aryl, heteroaryl, and heterocyclyl; and wherein any aryl- or heteroaryl-containing substituents of R^{c} are optionally substituted with one to three substituents independently selected from the group consisting of C₁₋₆alkyl, C₁₋₆alkoxy, halogen, fluorinated C₁₋₆alkyl, fluorinated C₁₋₆alkoxy, C₁₋₆alkylcarbonyl, C₁₋₆alkoxycarbonyl, aminocarbonyl, C₁₋₆alkylaminocarbonyl, di(C₁₋₆alkyl)aminocarbonyl, C₁₋₆alkoxycarbonylamino, formyl, C₁₋₆alkylsulfonyl, C₁₋₆alkylsulfonylamino, aminosulfonyl, C₁₋₆alkylaminosulfonyl, and di(C₁₋₆alkyl)aminosulfonyl, nitro, methylthio, hydroxy, and cyano; or, R^{c} and R^{d} are taken together with the atoms to which they are attached to form a 5-8 membered monocyclic ring that optionally includes 1 to 2 O or S heteroatoms within the ring, and said ring is optionally substituted with oxo;
with the proviso that in any instance, only one ring optionally exists between R^{a} and R^{b}, R^{b} and R^{c}, or R^{c} and R^{d}.

17. A method as in claim 16 wherein the pain is inflammatory pain.

18. A method as in claim 16 wherein the pain is visceral pain.

19. A method as in claim 16 wherein the pain is acute pain.

20. A method of treating or preventing pain comprising administering to a subject in need thereof, a pharmaceutical composition comprising a therapeutically effective amount of a compound of Formula (Ia) 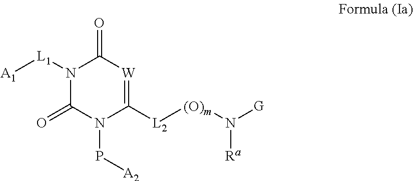
or an enantiomer, diastereomer, or pharmaceutically acceptable salt thereof;
wherein:
A₁ is phenyl or benzofuranyl; wherein phenyl is substituted at either the 4-position or 3- and 4-positions with one to two substituents independently selected from the group consisting of ethyl, methoxy, fluoro, chloro, nitro, difluoromethoxy, and methylthio;
L₁ is —CH₂— optionally substituted with methyl or allyl; P is —CH₂—;
A₂ is phenyl substituted at the para position with a substituent selected from the group consisting of methoxy, ethoxy, isopropyloxy, difluoromethoxy, hydroxy, and aminocarbonyl; or A₂ is pyridin-3-yl or pyridin-4-yl substituted with methoxy;
W is N or CH;
L₂ is a bivalent radical selected from the group consisting of
—NH-cyclohexyl-(CH₂)₀₋₂— and Q is attached at either the 2- or cis-4-position relative to the position of —NH—;
—X₁—CH(R^{x})—(CR^{y}R^{z})—; wherein X₁ is —NH— or S; and R^{x}, R^{y}, and R^{z} are each hydrogen;
and
—X₁—(R,R—CH(R^{x})CR^{y}(R^{z}))—; wherein X₁ is —NH—, and R^{x} and R^{z} are methyl, and R^{y} is hydrogen;
m is 0;
G is —C(═NR^{b})NR^{c}R^{d};
R^{a} and R^{d} are independently hydrogen, methyl or ethyl;
R^{b} is hydrogen;
R^{c} is hydrogen, C₁₋₄alkyl, C₂₋₄alkenyl, cyclohexyl, phenylcarbonyl, phenyl, pyrimidinyl, furanyl, benzo[1,3]dioxolyl, or pyridinyl; wherein C₁₋₄alkyl is optionally substituted with one to two substituents independently selected from the group consisting of C₁₋₃alkoxy, phenyl, pyridinyl, furanyl, and tetrahydrofuranyl; and wherein any phenyl- or heteroaryl-containing substituents of R^{c} are optionally substituted with one to two substituents independently selected from the group consisting of C₁₋₆alkyl, C₁₋₆alkoxy, chloro, fluoro, bromo, fluorinated C₁₋₃alkoxy, nitro, methylthio, hydroxy, and cyano; or, R^{c} and R^{d} are taken together with the atoms to which they are attached to form a 5-8 membered monocyclic ring.

21. A method of treating, ameliorating, or preventing pain, comprising administering to a subject in need thereof, a pharmaceutical composition comprising a therapeutically effective amount of a compound of Formula (I) 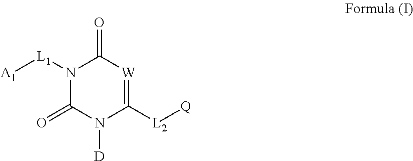
selected from the group consisting of:
a compound of Formula (I) wherein A₁ is phenyl, L₁ is —CH₂—, D is —CH₂-(4-fluoro-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methylcarboxy-phenyl), W is N, L₂ is —NH(CH₂)₂—, and Q is —NHC(═NH)NH₂;
a compound of Formula (I) wherein A₁ is phenyl, L₁ is —(CH₂)₂—, D is —CH₂-(4-methoxy-phenyl), W is N, L₂ is —NH(CH₂)₂—, and Q is —NHC(═NH)NH₂;
a compound of Formula (I) wherein A₁ is H, L₁ is —(CH₂)₄—, D is —CH₂-(4-methoxy-phenyl), W is N, L₂ is —NH(CH₂)₂—, and Q is —NHC(═NH)NH₂;
a compound of Formula (I) wherein A₁ is furan-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(3-trifluoromethyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-t-butyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-nitro-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is N, L₂ is —NH(CH₂)₂—, and Q is —ONHC(═NH)NH₂;
a compound of Formula (I) wherein A₁ is phenyl, L₁ is —CH₂—, D is —CH₂-pyridin-4-yl, W is N, L₂ is —NH(CH₂)₂—, and Q is —NHC(═NH)NH₂;
a compound of Formula (I) wherein A₁ is phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-difluoromethoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-n-butyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-trifluoromethyl-phenyl), W is N, L₂ is —NH(CH₂)₂—, and Q is —NHC(═NH)NH₂;
a compound of Formula (I) wherein A₁ is 2-fluoro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-trifluoromethoxy-phenyl), W is N, L₂ is —NH(CH₂)₂—, and Q is —NHC(═NH)NH₂;
a compound of Formula (I) wherein A₁ is 3-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 2-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-aminocarbonyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methylcarboxylamino-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —(R,R—CH(CH₃)CH(CH₃))—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —ONHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═N—CN)NH₂;
a compound of Formula (I) wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₄—, and Q is —NHC(═NH)NH₂;
a compound of Formula (I) wherein A₁ is 4-fluoro-phenyl, L₁ is —CH₂—, D is —(CH₂)₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-n-propyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-i-propyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-cyclopentyloxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methylthio-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-ethyl-phenyl), W is N, L₂ is —NH(CH₂)₂—, and Q is —NHC(═NH)NH₂;
a compound of Formula (I) wherein A₁ is 3-chloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-trifluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-difluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is cis-racemic-1,2-cyclohexyl, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is trans(1S,2S)-cyclohexyl-, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methylthio-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-ethyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is trans(1R,2R)-cyclohexyl-, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NH(3,5-dihydro-imidazol-4-on-2-yl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NH(4,5-dihydro-1H-imidazol-2-yl);
a compound of Formula (I) wherein A₁ is 3,4-dichloro-phenyl, L₁ is —CH₂—, D is —CH₂-(4-methylcarbonylamino-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-aminocarbonyl-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(3-ethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-ethoxy-phenyl), W is N, L₂ is —NH(CH₂)₂—, and Q is —NHC(═NH)NH-ethyl;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH-propyl;
a compound of Formula (I) wherein A₁ is 3,4-dichloro-phenyl, L₁ is —CH₂—, D is —CH₂-(4-methoxy-phenyl), W is N, L₂ is pyrrolindin-1-yl, and Q is 3-NHC(═NH)NH₂;
a compound of Formula (I) wherein A₁ is 4-chloro-phenyl, L₁ is —CH₂—, D is —CH₂-(4-methoxy-phenyl), W is N, L₂ is trans(1R,2R)-cyclohexyl-, and Q is —NHC(═NH)NH₂;
a compound of Formula (I) wherein A₁ is 3,4-dichloro-phenyl, L₁ is —CH₂—, D is —CH₂-(3-difluoromethoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(i-propyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —N(ethyl)C(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is 2-imino-imidazolid-1-yl;
a compound of Formula (I) wherein a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(n-butyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(cyclohexyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(benzyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(tetrahydrofuran-2-ylmethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenylethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(furan-2-ylmethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(2-methoxy-ethyl);
a compound of Formula (I) wherein A₁ is 3,4-dichloro-phenyl, L₁ is —CH₂—, D is —CH₂-(4-methoxy-phenyl), W is N, L₂ is —NH(CH₂)₃—, and Q is —NHC(═NH)NH₂;
a compound of Formula (I) wherein A₁ is 3,4-dichloro-phenyl, L₁ is —CH₂—, D is —(CH₂)₆—H, W is N, L₂ is —NH(CH₂)₃—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(allyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methoxy-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(NH)NH(4-chloro-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-trifluoromethyl-phenyl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(pyridin-3-yl);
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —NHC(═NH)NH(4-methylcarbonyl-phenyl);
a compound of Formula (I) wherein A₁ is furan-3-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is thiophen-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is R,S-mixture —CH(CH₃)—, D is —CH₂-(4-methoxy-phenyl), W is N, L₂ is —NH(CH₂)₂—, and Q is —NHC(═NH)NH₂;
a compound of Formula (I) wherein A₁ is 4-difluoromethoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-methoxy-phenyl), W is CH, L₂ is —NH(CH₂)₂—, and Q is —NHC(═NH)NH₂;
a compound of Formula (I) wherein A₁ is 4-methoxy-phenyl, L₁ is R,S-mixture —CH(allyl)-, D is —CH₂-(4-methoxy-phenyl), W is N, L₂ is —NH(CH₂)₂—, and Q is —NHC(═NH)NH₂;
a compound of Formula (I) wherein A₁ is 4-chloro-phenyl, L₁ is R,S-mixture —CH(allyl)-, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(6-methoxy-pyridin-3-yl), W is N, L₂ is —NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-cyclohexyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-nitro-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-(morpholin-4-yl)-eth-1-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—; and Q is —NHC(═NH)NH(3-(morpholin-4-yl)-prop-1-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-cyano-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-nitro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(1,3-benzodioxol-5-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHNH₂;
a compound of Formula (I) wherein A₁ is 3-nitro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-nitro-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3-amino-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-cyano-phenyl, L₁ is —CH₂—, D is —CH₂-(4-methoxy-phenyl), W is N, L₂ is v-NH(CH₂)₂—, and Q is —NHC(═NH)NH₂;
a compound of Formula (I) wherein A₁ is 3-cyano-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3-methoxycarbonyl-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-carboxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)C(Me)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-bromo-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyridin-2-yl-ethyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-ethoxycarbonyl-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2,4-difluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-decanyl);
a compound of Formula (I) wherein A₁ is 4-t-butoxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-hydroxy-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 2-chloro-thiazol-4-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is benzofuran-2-yl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxy-phenyl), W is N, L₂ is —NH(CH₂)₂—, and Q is —N(Me)C(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(CH₂CF₃);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(3-methoxypropyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)piperidin-1-yl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)N(Me)phenyl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-fluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-fluoro-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-methyl-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(t-butyl);
a compound of Formula (I) wherein A₁ is 4-chloro-phenyl, L₁ is —CH₂—, D is —CH₂-(4-amino-phenyl), W is N, L₂ is —NH(CH₂)₂—, and Q is —NHC(═NH)NH₂;
a compound of Formula (I) wherein A₁ is t-butyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is cyclopentyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is A₁ is 4-amino-phenyl, L₁ is —CH₂—, D is —CH₂-(4-methoxy-phenyl), W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(adamantan-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-trifluoromethoxy-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(4-hydroxy-phenyl);
a compound of Formula (I) wherein A₁ is 4-chloro-phenyl, L₁ is —CH₂—, D is —CH₂-phenyl, W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is 1,4-cyclohexyl, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NHCH₂C(═O)—, and Q is —NHC(═NC(═O)O-t-butyl)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(2-methylthio-phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(C(═O)phenyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(pyrimidin-2-yl);
a compound of Formula (I) wherein A₁ is 4-fluoro-phenyl, L₁ is —CH₂—, D is —CH₂-(4-methoxy-phenyl), W is N, L₂ is —NH((S)—CHMe)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH((R)—CHMe)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NH(═NH)NH(4-trifluoromethyl-5,6,7,8-tetrahydro-quinazolin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(5-methyl-pyridin-2-yl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)morpholin-4-yl;
a compound of Formula (I) wherein A₁ is 4-chloro-phenyl, L₁ is —CH₂—, D is —CH₂-furan-2-yl, W is N, L₂ is —NH(CH₂)₂—, and and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₅—, and and Q is —NHC(═NH)NH₂;
a compound of Formula (I) wherein A₁ is 4-methoxy-phenyl, L₁ is —CH₂—, D is —CH₂-(4-hydroxy-phenyl), W is N, L₂ is —NH(CH₂)₂—, and and Q is —NHC(═NH)NH₂;
a compound of Formula (I) wherein A₁ is 4-chloro-phenyl, L₁ is —CH₂—, D is —CH₂-(4-methoxy-phenyl), W is N, L₂ is —NH(CH₂)₆—, and Q is —NHC(═NH)NH₂;
a compound of Formula (I) wherein A₁ is 4-methoxy-phenyl, L₁ is —(CH₂)₂—, D is —CH₂-(4-methoxy-phenyl), W is N, L₂ is —NH(CH₂)₂—, and Q is —NHC(═NH)NH₂;
a compound of Formula (I) wherein A₁ is 4-methoxy-phenyl, L₁ is —(CH₂)₃—, D is —CH₂-(4-methoxy-phenyl), W is N, L₂ is —NH(CH₂)₂—, and Q is —NHC(═NH)NH₂;
a compound of Formula (I) wherein A₁ is 3,4-dichloro-phenyl, L₁ is —CH₂—, D is —CH₂-(4-methoxycarbonyl-phenyl), W is N, L₂ is —NH(CH₂)₂—, and Q is —NHC(═NH)NH₂;
a compound of Formula (I) wherein A₁ is phenyl, L₁ is —CH₂—, D is —CH₂-(4-n-butyloxy-phenyl), W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-phenyl, W is N, L₂ is —NH(CH₂)₂—, and Q is —NHC(═NH)NH₂;
a compound of Formula (I) wherein A₁ is 4-chloro-phenyl, L₁ is —CH₂—, D is —CH₂-furan-3-yl, W is N, L₂ is —NH(CH₂)₂—, and Q is —NHC(═NH)NH₂;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NHC(═O)methyl;
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(allyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(i-propyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(n-propyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(ethyl);
a compound of Formula (I) wherein A₁ is 4-fluoro-phenyl, L₁ is —CH₂—, D is —CH₂-(4-methoxy-phenyl), W is N, L₂ is —NH(CH₂)₂—, and Q is —NHC(═NH)NH(methyl);
a compound of Formula (I) wherein A₁ is 4-methoxy-phenyl, L₁ is —CH₂—, D is —CH₂-(4-methoxy-phenyl), W is CH, L₂ is —C(═O)NH(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is —O(CH₂)₂—, and Q is —NHC(═NH)NH₂;
a compound of Formula (I) wherein A₁ is 4-methoxy-phenyl, L₁ is —CH₂—, D is —CH₂-(4-methoxy-phenyl), W is CH, L₂ is —S(CH₂)₂—, and Q is —NHC(═NH)NH₂; and
a compound of Formula (I) wherein A₁ is 4-methoxy-phenyl, L₁ is —CH₂—, D is —CH₂-(4-methoxy-phenyl), W is CH, L₂ is —(CH₂)₃—, and Q is —NHC(═NH)NH₂.
